(19) United States
(12) Patent Application Publication
(10) Pub. No.: US 2007/0231819 A1
(43) Pub. Date: Oct. 4, 2007

(54) TARGETED AND NON-TARGETED GENE INSERTIONS USING A LINEAR MINIMAL ELEMENT CONSTRUCT

(76) Inventors: Christopher Lawrence, Blacksburg, VA (US); Yangrae Cho, Blacksburg, VA (US)

Correspondence Address: LATIMER IP LAW, LLP, 13873 PARK CENTER ROAD, SUITE 122, HERNDON, VA, 20171, US

(21) Appl. No.: 11/686,589

(22) Filed: Mar. 15, 2007

Related U.S. Application Data
(60) Provisional application No. 60/782,267, filed on Mar. 15, 2006.

(51) Int. Cl. C40B 30/06; C40B 40/08; C12N 1/26; C12N 15/74

(52) U.S. Cl. 435/6; 435/69.1; 435/254.1; 435/484

(57) ABSTRACT

The present invention provides nucleic acids and methods for disrupting genes within cells. The nucleic acids can be linear minimal elements that integrate into target cell genomes with high efficiency. The nucleic acids may be used to knock out specific genes or to increase expression of certain genes in a cell. Application in Alternaria fungi is exemplified.

### CROSS-REFERENCE TO RELATED APPLICATIONS

The present application relies on the disclosure of, and claims the benefit of the filing date of, U.S. provisional patent application No. 60/782,267, filed on 15 Mar. 2006, the entire disclosure of which is hereby incorporated herein by reference in its entirety.

### STATEMENT OF GOVERNMENT INTEREST

This invention was made partially with U.S. Government support from the United States National Science Foundation under Contract No. DBI-0443991. The U.S. Government has certain rights in the invention.

### BACKGROUND OF THE INVENTION

### Field of the Invention

The present invention relates to the field of molecular biology and bioinformatics. More specifically, the invention relates to nucleic acids and techniques using them for targeted disruption or overexpression of genes by introducing the externally-created nucleic acids into genes in a site-specific manner.

Complete genome sequences have been determined for over 100 model organisms, including the fungi Neurospora crassa, Aspergillus nidulans, and Candida albicans, to name a few. In addition, several plant-pathogenic fungal genome sequencing projects have been completed recently, including Magnaporthe grisea, Stagonospora nodorum, Sclerotinia sclerotiorum, and Fusarium graminearum. The completion of genome sequencing projects has led research communities to develop approaches and methodologies to explore gene function on a genome-wide scale. Characterization of gene expression patterns and transcriptional regulatory networks are common applications of functional genomics. In addition, disruption of several thousand genes is desirable as a part of functional analysis of individual genes or when identifying genes that contribute to a phenotype, such as plant pathogenicity.

The imperfect filamentous fungus Alternaria brassicicola causes black spot disease on a broad range of cultivated and weedy members within the Brassicaceae. Notably, A. brassicicola has been used as a true necrotrophic fungus for studies with Arabidopsis. Having genome sequences and functional methodologies developed for both plant and pathogen is advantageous for the elucidation of events occurring at the host-pathogen interface that ultimately determine the outcome of the interaction.

Targeted gene disruption or gene replacement, such as recombinatorial insertion of circular disruption constructs, recombinational replacement, or transposon arrayed gene knockout, is highly desirable for targeted mutational analysis in conjunction with a genome or large expressed sequence tag (EST) sequencing project. With the impending completion of the A. brassicicola genome sequencing project in late 2007, development of high-efficiency gene disruption technology for functional analysis is critical for the identification of virulence factors. Such technology also will be useful for identifying the functions of other genes of interest involved in a variety of biological processes.

Generation of gene disruption mutants is the most rate-limiting step for the functional analysis of individual genes in most filamentous fungi. Targeted gene disruption involves two separate processes: transformation of foreign DNA molecules into fungal cells, and their integration into the genome. Even after successful transformation, very inefficient integration of disruption constructs is quite common among plant-associated filamentous fungi. For example, fewer than 1% of transformants were targeted gene disruption mutants in Septoria lycopersici when attempts were made to disrupt a tomatinase gene, and in Acremonium chrysogenum when attempts were mad to disrupt a transcription factor (Martin-Hernandez et al. 2000; Schmitt et al. 2004). In contrast, Alternaria alternata has been shown to exhibit exceptionally high efficiency for transformation and targeted gene disruption by homologous recombination. Approximately 90 transformants were generated with 1 ug of plasmid construct that contained a 3-kb-long rDNA cassette sequence that is known to repeat over 200 times in the genome (Tsuge et al. 1990). Meanwhile, targeted gene disruption efficiency approached 100% for a melanin biosynthesis gene using linearized disruption constructs containing as short as 600 bp of target sequence (Shiotani and Tsuge 1995).

A. brassicicola, in contrast to studies with other Alternaria species, previously has been reported to exhibit very low efficiency for both transformation and targeted gene disruption. Yao and Koller (1995) reported a rare case of gene disruption in A. brassicicola for a cutinase gene cutab1. In this study, high-velocity microprojectiles (delivered via gene gun) were used to introduce circular plasmid constructs harboring a partial cutab1 cDNA into conidia. Two targeted gene mutants were identified out of 30 hygromycin B (hygB) resistant transformants, whereas a polyethylene glycol (PEG)-mediated protoplast transformation method failed to generate any transformants.

Thus, there exists a need in the art for improved techniques and constructs for disruption or expression of individual target genes in fungi. The need extends to techniques and constructs that are widely applicable and easy to use. Further, there is a need for constructs that are easy to design and create and have high transformation and integration efficiencies, and that are accordingly useful in high-throughput screening for fungal mutants and development of new fungal strains for use in industry and for scientific study. Preferably, these techniques and constructs would be applicable to other organisms and species.

### SUMMARY OF THE INVENTION

The present invention addresses needs in the art. It provides a nucleic acid construct for efficient, targeted disruption or expression of genes of interest in fungi, and in particular, species of Alternaria. It further provides a technique for targeted disruption or expression of genes of interest, such as a method of high-throughput screening for cells lacking a functional target gene, or cells overexpressing a target gene. Although applicable to many species, it is particularly useful in species of Alternaria, such as A. brassicicola.

In a first aspect, the invention provides nucleic acids. The nucleic acids comprise one or more engineered or heterologous cassettes comprising, in any order, a linear sequence of elements comprising: a nucleotide sequence of at least a portion of a target gene; and at least one nucleotide sequence having a known sequence, length, or other detectable characteristic. In embodiments, the nucleotide sequence has a known restriction endonuclease cleavage pattern. In other embodiments, the nucleotide sequence encodes at least one detectable product. Among other things, the nucleic acids of the invention find use in targeted disruption of particular genes of interest for study of the genes and their roles in development, growth, and/or death of the organism in which they are found. In addition, the nucleic acids of the invention further find use in, for example, overexpression of cellular genes for study of the genes (and, more typically, the gene products) in development, growth, and/or death of the organism in which they are found.

In another aspect, the present invention provides compositions comprising one or more nucleic acids of the invention, cells containing at least one nucleic acid of the invention, and kits comprising at least one nucleic acid of the invention. While not limited in their use, the compositions and kits can be used for practicing a method of the invention.

In yet another aspect, the invention provides a method of inserting a heterologous nucleic acid into a host genome. In general, the method comprises: integrating some or all of a nucleic acid of the invention into a host cell genome; subjecting the host cell to conditions that permit expression of the nucleotide sequence encoding the detectable marker; and determining whether the detectable marker has been expressed. In embodiments, the method further comprises providing the nucleic acid of the invention. The method may also further comprise introducing the nucleic acid into the target cell. Introducing may be by any means, including, but not limited to, transformation, transfection, electroporation, through the use of a “gene gun”, and the like. Exemplary embodiments of the method provide for expression of a heterologous nucleic acid inserted into a host genome by homologous recombination of some or all of a nucleic acid of the invention. Other exemplary embodiments provide for expression or overexpression of a gene naturally present in the genome of the host organism, but under the control of a control element that is heterologous to the gene and has been introduced by way of homologous recombination of at least part of a nucleic acid of the invention. Yet other exemplary embodiments provide methods for reducing or eliminating (e.g., knocking-out or silencing) the expression of a host gene in a host genome by inserting a nucleic acid of the invention into the expression control region or coding region of the gene. Thus, the invention provides a method of altering the expression of a host gene in a host genome.

In a further aspect, the invention provides a method of producing a protein in a host organism. In general, the method comprises inserting, by homologous recombination, some or all of a nucleic acid of the invention into the genome of a host organism, and expressing a protein as a result of the insertion. The protein may be a protein naturally encoded by the genome of the organism or may be heterologous to the host genome. Expression may be from a natural promoter present in the host genome or by way of one or more heterologous nucleic acid sequences provided on the nucleic acid of the invention. Accordingly, the present invention provides an expression platform for production of proteins of interest, particularly those expressed in fungal cells, such as those of the genus Alternaria.

In yet a further aspect, the invention provides methods for identifying genes and proteins having a detectable effect on cells. For example, the method can be a method of high-throughput screening for genes having certain effects on the growth, maintenance, and/or environmental activity of cells. In embodiments, the invention provides methods of high-throughput screening for genes and gene products involved in pathogenesis of fungi. Among other things, it also provides methods for screening for genes and gene products that have toxic or other effects on other organisms, such as on plants.

### BRIEF DESCRIPTION OF THE DRAWINGS

The accompanying drawings, which are incorporated in and constitute a part of this specification, illustrate several embodiments of the invention, and together with the written description, serve to explain certain principles of the invention.

FIG. 1 is a diagram depicting incorporation of transforming DNA at a target genomic locus. Panel A shows integration using a circular construct. Panel B shows integration and gene replacement using either a circular or linear construct. Panel C shows integration using a Linear Minimal Element (LME) of the present invention.

FIG. 2 shows constructs and corresponding gel electrophoresis results of nucleic acids according to the invention. Panel A shows on the left a schematic of gene disruption resulting from LME (top and middle), and wild type genomic locus (bottom) for disruption of the chymotrypsin gene, and on the right the corresponding nucleic acids from PCR reactions. Panel B shows on the left a schematic of gene disruption resulting from LME (top, middle, bottom), for disruption of the N-acetylglucosaminidase gene, and on the right the corresponding nucleic acids from PCR reactions. Primers used to verify gene disruption and amplification of the wild type locus if applicable are depicted as arrows on the left hand schematics in both A and B.

FIG. 3 shows a restriction map and Southern blot of constructs of the invention and incorporation into target cellular gene N-acetylglucosaminidase. Panel A shows a restriction map of the gene (upper portion), an LME construct (middle portion), and the resulting disrupted gene (bottom portion). Panel B shows a Southern blot analysis of mutant genes resulting from disruption in individual fungal transformants and the wildtype fungus.

FIG. 4 shows schematic diagrams of targeted gene disruption with a representative linear minimal element construct.

FIG. 5 shows expression of green fluorescent protein (GFP) in cells having an N-acetylglucosaminidase gene disrupted (upper portion) by an LME (bottom portion).

FIG. 6 shows bar graphs indicating overexpression of a gene from a construct of the invention. Panel A depicts the construct. Panel B depicts expression levels in six transformants in A. brassicicola. Panel C depicts expression levels in eight transformants in A. alternata.

FIG. 7 depicts a schematic representation of events occurring in targeted gene disruption by a single crossover homologous recombination event with an LME construct of the invention.

FIG. 8 shows constructs (Panel A) and Southern blot analyses (Panel B) of a Linear Minimal Element (LME) of the invention.

FIG. 9 shows the effects of LME integration and abnps2 mutation on conidia water absorption. Panels A-C show absorption of water by wild-type conidia (Panel A), an LME disruption mutant of abnps2 (Panel B), and an ectopic mutant (Panel C). Panels D and F show transmission electron micrographs (TEM) depicting cell wall structures of wild-type A. brassicicola conidia. Panels E and G show similar TEM, but taken of abnps2 mutants.

FIG. 10 shows the effects of LME integration on virulence. Panel A shows the effect of mutation of abnps2 on conidiation. Panels B and C show germination efficiency on green cabbage in vitro (Panel B) and in vivo (Panel C). Panel D depicts pathogenicity of wild-type, ectopic, and abnps2 mutant strains of A. brassicicola on cabbage leaves. Panel E graphically depicts the results shown in Panel D. Panels F and G depict electron micrographs of 21-day old wild-type (Panel F) and abnps2 mutant (Panel G) conidia.

FIG. 11 shows integration of a circular LME construct of the invention into the MAP kinase gene of A. brassicicola (Panel A). Panel B shows Southern blot analyses of mutants created. Panel C shows agarose gel analysis of PCR products derived from fungal transformants in which the wild-type Amk1 gene was reintroduced into the amk1 mutant.

FIG. 12 shows the effects of targeted disruption of the Amk1 locus on fungal morphology and virulence. Panel A shows terminal structures leading to conidia production (left side—typical; right side—amk1-c mutants). Panel B shows conidial chains (left side—typical; right side—amk1-c mutants). Panel C shows ultrastructural interfaces between host plant and fungi during early infection (left side—typical; right side—amk1-c mutants).

FIG. 13 shows that amk1 mutants have lower infectivity than wild-type organisms.

FIG. 14 shows the effect of plant wounding and nutrient supplementation on infectivity of amk1 mutants. Panel A shows the difference in infectivity of a mutant on an intact surface (“a/in”) and a wounded surface (“a/wd”). Panel B shows the difference in infectivity of a mutant on a wounded surface (“a/wd”) and a wild-type organism on an intact surface (“wt/in”). Panel C shows the differences in infectivity in the presence of glucose (glu), casamino acids (ca), yeast extract (ye), tryptone (trp), and bovine serum albumin (bsa).

FIG. 15 shows the effects of nutrients on growth in vivo of amk1 gene mutants. Panel A shows growth of wild-type or amk1-c mutants on plates containing various nutrients. Panel B depicts the growth shown in Panel A in graphical form.

FIG. 16 shows bar graphs depicting the role of Amk1 in the regulation of hydrolytic enzyme gene production. Shown are quantitative reverse-transcriptase PCR (QRT-PCR) results for the expression of actin, Alt2b, and six hydrolytic enzyme genes.

FIG. 17, Panels A-D, show nucleic acid constructs (Panel A), mechanism of insertion into host genome (Panel B), and assays for production of recombinant protein (Panels C and D) for introduction of a histidine tag at the C-terminus of a protein of interest, and expression of the protein in a host organism.

### DETAILED DESCRIPTION OF VARIOUS EMBODIMENTS OF THE INVENTION

Reference will now be made in detail to various exemplary embodiments of the invention, examples of which are illustrated in the accompanying drawings. The following detailed description is provided to give the reader a more detailed understanding of certain features of the invention. As such, it is not to be understood as a limitation of the scope or subject matter of the invention.

In a first aspect, the invention provides nucleic acids (also referred to herein as “constructs”). In general, the nucleic acids of the invention are relatively simple constructs that provide one or more sequences for integration of the nucleic acids into host genomes, and one or more sequences that provide an ability to detect integration of the nucleic acids into host genomes. The nucleic acids thus can comprise one or more engineered or heterologous cassettes comprising, in any order, a linear sequence of elements comprising: a first nucleotide sequence of at least a portion of a target nucleic acid, such as a gene; and a second nucleotide sequence having a known sequence, length, or other detectable characteristic. In embodiments, the second nucleotide sequence has a known restriction endonuclease cleavage pattern. In other embodiments, the second nucleotide sequence encodes at least one detectable product. The nucleic acid of the invention can independently comprise two or more of each of the first and second sequences.

It is to be understood that, unless otherwise noted, reference to one strand of a nucleic acid or a particular nucleotide sequence inherently implies a naturally occurring other, complementary strand. Accordingly, reference to a nucleotide base in the context of a naturally occurring double-stranded nucleic acid molecule implies its base pair as well.

The nucleic acids of the invention may be any type of nucleic acid known. Thus, they may be DNA or RNA or any modified form of these (e.g., PNA). They may be double stranded, single stranded, or even triple stranded. The size of the nucleic acid can be any size that is appropriate for the purpose of the nucleic acid. Thus, where the nucleic acid is a cassette for insertion into another nucleic acid, such as a host cell genome, it may be as small as several hundred nucleotides or as large as several kilobases. Similarly, where it is a plasmid or other vector, it may be hundreds or thousands of bases in size. Likewise, where the nucleic acid is a cellular genome (including those with stable extra-chromosomal elements) comprising a construct of the invention, the nucleic acid may be hundreds, thousands, or millions of kilobases. As there is significant overlap between sizes of known vectors and cellular genomes, there is no particular range for any specific type of nucleic acid. It is sufficient to recognize that the present invention encompasses nucleic acids of all sizes and functions, having only naturally occurring bases and base pairing, or having one or more unnatural bases, linkages, or base pairings.

The nucleic acids of the invention comprise a nucleotide sequence of at least a portion of a target gene. The length of the target gene present on the cassette is not particularly limited except that it must be of sufficient length to participate in homologous recombination. For example, it could be about 1 kb or more. Alternatively, it could be less than 1 kb, such as 500 bases, 250 base, 100 bases, or fewer. The target gene sequence may be present at any region of the nucleic acid, including at an end or in the center of the nucleic acid cassette. In addition, the target gene sequence that is present on the cassette may be modified by introduction of nucleotides of interest, which can aid in determining the presence of the cassette in a host genome (e.g., an engineered restriction site). Preferably, any engineered nucleotide does not alter the naturally encoded amino acid sequence or does not negatively affect expression of the target gene. It is to be understood however that, in embodiments, alterations to an endogenous host cell gene are intended by integration of a nucleic acid of the invention into the host genome, and such alterations can be by simple alteration of one nucleotide in the target sequence.

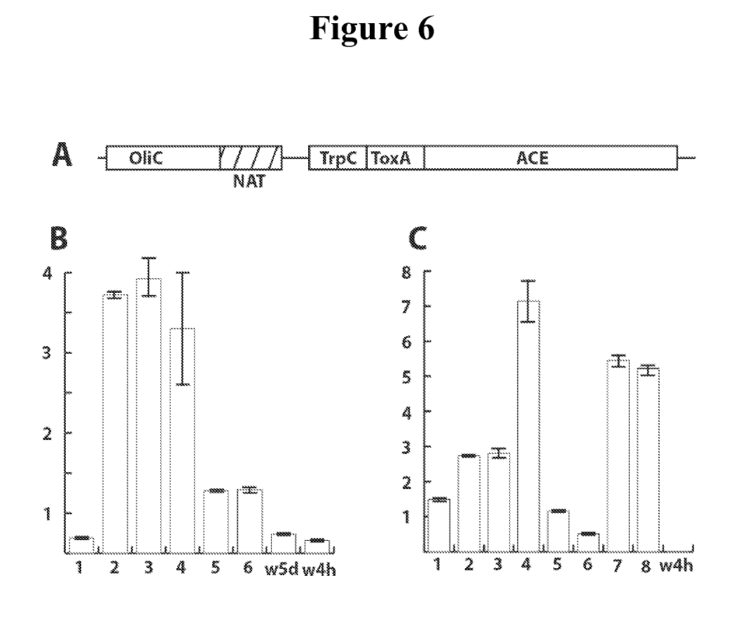

The target gene may be any gene of interest that is present in the genome of an organism of interest. Thus, the gene may be, for example, a gene that is involved in growth and/or development of an organism. It also may be, for example, a gene that is involved in homeostasis of a cell in a certain environment, such as genes that are commonly referred to as “housekeeping” genes. In some embodiments, the gene is a gene involved in pathogenicity of the organism. Alternatively, the gene may be one that is involved in communication with other organisms, such as when the cell is in close contact with other cells (of the same type or from another species) in a multicellular collection of cells. In yet other examples, the gene may be one involved in protecting a cell from environmental hazards or toxic compounds, or in the production of compounds that are toxic to one or more other organisms (e.g., toxins, antibiotics). In essence, the target gene may be any gene of interest to one practicing the invention, whether the interest be for research purposes or industrial purposes. In some embodiments, the identity of the target gene and it's encoded protein is not known. That is, the portion of the nucleic acid of the invention that is homologous to a sequence in a genome of an organism may be randomly selected or generated, such as, for example, when preparing a library of LME cassettes for high-throughput screening (see below). Accordingly, the LME of the invention can be used for non-targeted insertion of nucleic acid sequences into an organism's genome. In these situations, integration might occur either by homologous recombination or by other mechanisms. A non-limiting example of such a mechanism is provided below with regard to FIG. 6.

The target gene is a gene present in an organism of interest. An organism of interest is also referred to herein at times as a host cell, particularly after having had the nucleic acid of the invention introduced into it, either actively through action of a human, actively through its own ability to take up nucleic acids, or any other way known. According to the invention, an organism of interest may be any organism from any of the three main branches of life (eukaryotes, eubacteria, archaea). It thus may be a eukaryotic organism or a prokaryotic organism. Non-limiting examples of organisms include, but are not limited to “lower” eukaryotic organisms, such as yeasts and other fungi. In exemplary embodiments, the organism is a fungus from the Deuteromycota, such as an Alternaria species. A. brassicicola and A. alternata are two representative species discussed in the Examples, although any other fungal species is equally relevant to the present invention.

The nucleic acid of the invention further comprises at least one nucleotide sequence having a known physical or functional characteristic. The physical characteristic can be any one that can be determined, including, but not limited to, size, restriction endonuclease cleavage pattern, base pair composition, sequence (including the sequence of one or more portions of the total nucleotide sequence), presence of distinctive secondary or tertiary structure(s), and presence of detectable labels. The functional characteristic can be any one that can be determined, including, but not limited to, activity of an encoded protein, ability to hybridize with another nucleic acid molecule, and ability to specifically interact with a protein. Of course, the nucleotide sequence may have more than one physical characteristic, more than one functional characteristic, of a combination of one or more physical and/or functional characteristics.

In embodiments, the nucleotide sequence encodes at least one detectable product. For example, in embodiments, the nucleotide sequence encodes a single detectable product. The detectable product can be any product that is detectable by any means. Thus, it can be a protein having an intrinsic property that is detectable, such as the fluorescence of green fluorescent protein (GFP) or blue fluorescent protein (BFP). It can also be a protein having an activity that results in a detectable product, such as an enzyme that produces a detectable product (e.g., luciferase). Alternatively, it may be a product that has a biochemical property that can be detected, such as antibiotic resistance or resistance to some other toxic compound. Other non-limiting examples of detectable products include those that provide a growth advantage for a cell, those that are necessary for growth or life of a cell, and those that are normally present in a cell, but toxic to the cell when present in excess. As can be seen, it is not necessary that the nucleic acid encode a product that is detectable, per se. Rather, the nucleic acid can encode a product that is detectable through inference of the activity of the product, such as by production of a detectable signal through its enzymatic activity or through its participation in a simple or complex biochemical process.

In embodiments of the invention, the nucleic acid is a double-stranded DNA molecule. The molecule can be present as a linear molecule or as a circular molecule. In some cases, it may be provided as a linear molecule, but later circularized, either as a natural result of its intrinsic properties, or, more commonly, through the action of one or more cellular components. In embodiments, the nucleic acid is provided in a linear form having a particular sequence, but is later concatamerized to produce a linear (or, ultimately, circular) molecule having two or more copies of the particular sequence. Where multiple copies of the particular sequence are present on a single molecule, the orientation of the sequences may be mixed. That is, all of the sequences can be in the same orientation (with regard to positioning of elements from one end to the other along the linear molecule), or some may be in one orientation while others are in the other orientation. The molecule may comprise various elements typically found on nucleic acid molecules used in molecular biology technologies, such as sequences for replication or maintenance of the molecule in a cell, sequences for encapsulating the nucleic acid in a protein or membrane-containing package (e.g., sequences encoding viral coat proteins, etc.), and the like. Those of skill in the art are well aware of such sequences, and may select desirable ones and incorporate them into the nucleic acid of the invention without undue experimentation.

In embodiments, the nucleic acid is a linear minimal element comprising: a first nucleotide sequence that is homologous to a nucleotide sequence of a target gene in a target cell genome; and a second nucleotide sequence having a sufficient length and sequence to disrupt the coding sequence of the target gene. The first and second nucleotide sequences can be linked by way of one or more intervening nucleotides. For example, a linker of about 10 to about 100 nucleotides may be present between the first and second nucleotide sequences. In some embodiments, the first nucleotide sequence is positioned 5′ (“upstream” with regard to expression of the homologous sequence in the target gene) of the second nucleotide sequence, while in other embodiments, the second nucleotide sequence is positioned 5′ of the first nucleotide sequence. In some embodiments, both sequences are oriented in the same direction (where the second sequence encodes a product), while in other embodiments, the second sequence is in the opposite orientation to the first. Where multiple sequences are present, they can be arranged in any suitable orientations with respect to each other. In a similar construct for the same general purpose, the linear minimal element comprises a target sequence that targets a sequence within the control region (e.g., promoter, activator binding site, repressor binding site, transcription factor binding region, etc.) of a gene, where the target sequence is sufficiently homologous over at least a portion to participate in homologous recombination. The linear minimal element also comprises a sequence that is homologous to the target sequence, but has one or more nucleotide deletions, substitutions, or additions, which reduce or abolish the function of the control region, resulting in reduction or loss of expression of the coding region that is operably linked to the control region.

In other embodiments, the nucleic acid is a linear minimal element comprising: a first nucleotide sequence that is homologous to a nucleotide sequence of a target gene in a target cell genome; and a second nucleotide sequence having a sufficient length and sequence to disrupt the control region of the target gene to result in increased expression of the target gene. For example, some or all of the control region can be replaced by a heterologous control region that is more active or under different (or no) control, as compared to the naturally-occurring region. In this way, integration of the cassette into the host genome can alter, and preferably increase, the expression of the targeted gene. In embodiments, the linear minimal element comprises sequences that are binding sites for proteins or small molecules, the binding of which to the control region being easily regulated by environmental conditions (e.g., supplying compounds to the cell, altering temperature of growth, and the like). Whereas the constructs discussed in the previous paragraph may be typically used for knock-down or knock-out of gene expression, the constructs discussed in this paragraph may be typically used for over- or hyper-expression of genes.

Where the nucleic acid of the invention is a construct for expression of a gene, whether the gene be a heterologous gene (e.g., encoding a detectable product) or an endogenous gene present in the genome of the target host cell, the nucleic acid can comprise one or more expression control elements. For example, it can contain a promoter and regulatory elements for binding of repressors, derepressors, transcription factors, and the like. It can contain polymerase binding sites, sequences that signal termination of transcription, and the like. It further may encode amino acid sequences for ribosome binding and translation control. Any number and combination of elements may be included in the construct.

Accordingly, in embodiments, the nucleic acid is a linear minimal element that comprises: a sequence for homologous recombination into a host cell genome; and a coding region for a protein of interest, operably linked to a control region that directs expression of the protein of interest. The sequence for homologous recombination can be specific for a gene of interest (resulting in both disruption of the target gene and expression of the newly provided gene), or can be specific for a region of the host genome known or thought to be a non-coding region. Of course, the protein of interest can be a protein naturally produced in the host cell (an endogenous protein) or a protein that is not naturally produced in the host cell (a heterologous protein). It is to be noted at this point that, unless otherwise stated, the term “protein” includes all poly-amino acid molecules, including those that are commonly referred to as peptides or polypeptides. Because the linear minimal element of these embodiments comprises a coding sequence of interest operably linked to a control region of interest (both of which may be engineered to have desirable qualities), integration of the cassette into a host genome can provide a platform for production of the protein of interest.

In preferred embodiments, the nucleic acid is a nucleic acid that is capable of circularization to form a closed circle molecule. The circular molecule may comprise all of the nucleotides of the original linear element, or may comprise fewer, which may result from exonucleolytic digestion of one or both ends of the linear molecule. Thus, the invention provides a nucleic acid molecule that is a closed circular molecule comprising two nucleotide sequences: a first sequence that is homologous to a target sequence in a genome of a target organism; and a second sequence that contains a sequence that has sufficient physical or functional characteristics to disrupt the target sequence. In embodiments, the second sequence comprises one or more transcriptional control elements (e.g., a promoter) that permit expression in the target cell of a product encoded by the second sequence. In other embodiments, the second sequence comprises one or more transcriptional control elements that permit expression of the coding region of a gene, or a portion of the coding region of a gene, of the target organism. Other embodiments are discussed in the context of the basic linear minimal element, above.

Thus, the invention provides nucleic acid constructs for expression of cellular genes in a host organism, from the host genome. These constructs may comprise a first sequence encoding a detectable marker, such as an antibiotic resistance marker, a second sequence that is homologous to a sequence of a target gene in a target organism, and optionally a third sequence, which is a transcriptional control element operably linked to the second sequence. In some embodiments, two or more transcriptional control elements are operably linked to the second sequence. As used herein, a sequence is operably linked to another if it functions to affect the expression of the other. While it is preferably that the two sequences be physically linked, for example by being present on the same nucleic acid molecule, such a physical linkage is not necessary.

As should be evident, the nucleic acid of the invention can be a linear minimal element. Alternatively, it can be a circular element that is incapable of self-replication. Yet again, it can be a plasmid or other vector that is capable of integrating into a host genome or self-replicating to maintain itself in host cell as an integrated element in a host chromosome or as an autonomously replicating element, respectively. In addition, it can be a chromosome of a cell, which contains the nucleic acid as a heterologous insertion. The heterologous insertion may be stable or transient.

Among other things, the nucleic acids of the invention find use in targeted disruption of particular genes of interest for study of the genes and their roles in development, growth, and/or death of the organism in which they are found. In addition, the nucleic acids of the invention further find use in, for example, overexpression of cellular or heterologous genes for study of the genes (and, more typically, the gene products) in development, growth, and/or death of the organism in which they are found. Of course, the nucleic acids can be used to create expression platforms in host cells for production of proteins of interest (e.g., for research or commercial purposes). Nucleic acids could also be used to increase the amounts of specific secondary metabolites via homologous recombination-based promoter swapping or overexpressing genes such as transcription factors that regulate the production of secondary metabolites, chemicals, or pharmaceutical preparations.

In another aspect, the present invention provides compositions comprising one or more nucleic acids of the invention. In general, the compositions comprise, in addition to the nucleic acid, a liquid or solid. For example, the composition can comprise water, an organic solvent, or both. Thus, the composition may comprise a substance that allows the nucleic acid to be present in a liquid composition, such as a solution or mixture. In addition or alternatively, the composition may comprise a salt or other solid substance. For example, it may comprise a salt that assists in solubilizing the nucleic acid or maintaining the nucleic acid in solution. The type of salt is not particularly limited, and non-limiting examples include salts comprising Sodium, Magnesium, Manganese, Lithium, Potassium, Chlorine, Calcium, acetate, and phosphate. The composition comprising the nucleic acid thus may be a liquid composition or a solid composition. Of course, the composition may comprise water, and thus may be a composition comprising liquid water or a composition comprising ice. In some embodiments, the composition comprises two or more nucleic acids of the invention. It is to be noted that “a” nucleic acid and compositions described as comprising “a” nucleic acid of the invention may comprise one or more identical copies of a nucleic acid having the same nucleotide sequence. In contrast, compositions comprising more than one nucleic acid are to be understood to contain a combination of nucleic acids, in which two or more nucleic acids having different nucleotide sequences are present. The number of different sequences and the number copies of each nucleic acid are not limited, and can range from one to millions or more. As evidenced below, compositions of the invention can be those that are suitable in practice of at least one portion of at least one method of the invention.

In exemplary compositions, the compositions comprise a sufficient amount of one or more nucleic acids to insert the nucleic acid into a target site on a target nucleic acid (e.g., into a host genome) or a sufficient amount to analyze (e.g., to detect using PCR or restriction endonuclease digestion and agarose gel electrophoresis). In embodiments, there is a sufficient amount for use in one or more molecular biology protocols (e.g., for subcloning into a vector for expression or amplification). In some embodiments, the compositions comprise one or more lyophilized nucleic acids of the invention. In yet other embodiments, the compositions comprise agarose, polyacrylamide, or another matrix-forming compound that is useful in analyzing nucleic acids.

In some embodiments, the compositions comprise, in addition to a nucleic acid of the invention, some or all of the reagents and other substances that are suitable for introduction of the nucleic acid into a host cell, and preferably into a host genome. Thus, the compositions may comprise one or more buffers in an aqueous solution. Alternatively, they may comprise substances that assist in introducing nucleic acids into cells, such as gold or tungsten particles. In other embodiments, the compositions comprise reagents and other substances that are suitable for analysis of nucleic acids, such as, but not limited to, polymerases for amplification of the nucleic acids, dyes for detecting nucleic acids, enzymes for cleaving or labeling nucleic acids, nucleotides (e.g., for sequencing or amplification of nucleotide sequences), salts, and the like.

In another aspect, the invention provides cells. In general, the cells may be any cells into which nucleic acids can be introduced or maintained. The cells thus may be prokaryotic or eukaryotic. In preferred embodiments, they are eukaryotic cells, and more specifically, fungal cells. Among the fungal cells envisioned by this invention, cells of the genus Alternaria are preferred. Among the Alternaria, mention can be made of A. brassicicola and A. alternata. An exemplary organism is A. brassicicola, which shows certain advantageous qualities for introduction into its genome of nucleic acids according to the present invention. According to the invention, the cells are typically cells into which one or more nucleic acids of the invention have been introduced. The nucleic acids of the invention are preferably integrated, partially or wholly, into the genome of the cell to create a recombinant cell. It is preferred that the nucleic acids, and thus the recombinant cells, comprise a heterologous nucleic acid sequence, although this is not a required feature of the invention. In accordance with the discussion above, recombinant cells of the invention may comprise a nucleotide sequence that encodes a detectable marker (e.g., GFP) or intrinsically has a detectable feature (e.g., a restriction digestion pattern).

Cells of the invention have many uses, not the least of which is as an expression vehicle for expression of engineered proteins. More specifically, nucleotide sequences that are introduced into a host cell genome, whether those sequences are coding sequences from the same or another organism, or are control regions from the same or another organism, can be used to express a gene product of interest. Where the gene product is a heterologous protein, expression can result in production of the protein in the host cell. Alternatively or in addition, where the introduced nucleotide sequence is or is part of an expression control region, expression of the gene product can be controlled. For example, expression of an endogenous gene product can be altered, preferably increased, in the recombinant cell to produce large quantities of the gene product. In addition, expression of a heterologous gene product can be placed under the control of a naturally-occurring control region, the natural control region for the heterologous gene product can be introduced into the cell with the heterologous gene coding sequence, or an engineered control region can be introduced into the host cell genome with the heterologous gene coding sequence. Where the host cell is an Alternaria species, such as A. brassicicola, the cell can be an alternative expression platform to other fungal platforms. It is to be understood that one could use LME constructs delivered into fungal protoplasts, spores, or hyphae (mycelia); thus, there is no limitation on the developmental stage of the organism. Of course, the invention provides compositions comprising cells of the invention.

In yet another aspect, the invention provides a method of inserting a heterologous nucleic acid into a host genome. In general, the method comprises: contacting a host cell with at least one nucleic acid of the invention under conditions that allow for insertion of the nucleic acid(s) into the cell; providing conditions that allow for integration of some or all of the nucleic acid(s) into the host cell genome. Although not limited in its molecular mechanism, integration of the nucleic acid is preferably by homologous recombination. In exemplary embodiments, integration is by a single crossover homologous recombination event.

According to the method, contacting can be any action that results in at least one nucleic acid molecule physically contacting at least one host cell. It thus may be simply by way of exposing the two to each other in a contained environment, such as in a reaction tube or the like, for an adequate amount of time for random diffusion and Brownian movement to cause the two to physically contact each other. Alternatively, the nucleic acid and/or host cell can be actively targeted to each other, for example by way of a delivery vehicle that comprises the nucleic acid and specifically binds to a molecule on the surface of the host cell. Then again, the two may be actively contacted by way of routine procedures known in the art for introducing nucleic acids, such as double-stranded DNA, into eukaryotic cells, and in particular, fungal cells. Exemplary methods for contacting nucleic acids and fungal cells are provided in the Examples, below. The method thus may comprise introducing the nucleic acid into a target cell (i.e., a host cell). Introducing may be by any means, including, but not limited to, transformation, transfection, electroporation, through the use of particle bombardment (“gene gun”), delivery using Agrobacterium tumefaciens, and the like.

According to the method, conditions are provided that allow for integration of the inserted nucleic acid into the host cell genome. While not limited in any way to a particular means of providing conditions, in typical embodiments, cells comprising a nucleic acid of the invention are incubated for a time that is adequate for cellular machinery and processes to integrate the introduced nucleic acid into the host genome.

In many situations, the practitioner will wish to confirm that the nucleic acid was integrated at the site of the host genome desired (i.e., successfully integrated at the target site). In those situations, the method can comprise determining if some or all of the nucleic acid integrated into the host genome. Such determining can be accomplished by numerous methods. Examples include, but are not limited to: isolating genomic DNA and digesting it with selected restriction endonuclease(s) to identify a signature restriction pattern; isolating genomic DNA and amplifying a known sequence of the inserted nucleic acid and surrounding genomic DNA to determine if it is present and if so, where; probing genomic DNA (either in the cell or in an isolated form) with one or more sequence-specific probes to determine if, where, and how many times, the nucleic acid was integrated into the host genome.

In other situations, the practitioner may simply wish to confirm that the nucleic acid was integrated into the genome in a way that allows for expression of at least one coding sequence on the nucleic acid. In these situations, the practitioner may use the techniques discussed immediately above, or may simple assay the cell (intact or after lysis) for the gene product of interest. This can be by any means known in the art, and will typically be by: growth in the presence of a toxic compound (e.g., an anti-fungal compound); growth in the absence of a nutrient that is typically required by the host cell for growth; production of an enzyme having a known activity (e.g., luciferase); production of a protein having an intrinsic detectable characteristic (e.g., GFP); and detection of a protein to which antibodies are available. Thus, in these embodiments, the method may comprise detecting the presence of an integrated form of the coding sequence of interest by subjecting the host cell to conditions that permit expression of at least part of the introduced nucleotide sequence. It thus can comprise determining whether a detectable marker encoded on the introduced nucleic acid is expressed in the recombinant cell.

The methods of the invention can be methods for transformation of a target cell. When practiced in this way, the methods can be methods for disruption of a target gene. Disruption can be for the purpose of reducing or eliminating expression of one or more target genes, for expression of one or more heterologous genes, and/or for expression of an endogenous gene. Thus, disruption indicates insertion of heterologous sequences into a target host genome sequence, but does not necessarily imply a deleterious effect on expression. Furthermore, the methods of the invention provide for high efficiency transformation of cells, and in particular fungal cells such as cells of the Alternaria genus, including but not limited to A. brassicicola, and for high efficiency of integration of the construct into genomes. It is especially and surprisingly suitable for extremely high efficiency of integration at specific sites, consistently providing 70% or higher efficiencies in A. brassicicola. For example, it can provide about 75% or higher, about 80% or higher, about 85% or higher, about 90% or higher, about 95% or higher, about 98% or higher, about 99% or higher, or about 99.5% or higher efficiency. Of course, those of skill in the art will immediately recognize that any particular value within this range of numbers is encompassed by the invention, without the need to specifically recite each value herein. The surprising result of high efficiency is achieved in contrast to prior attempts at integration in fungal cell genomes using linear constructs and circularized constructs for homologous recombination.

As a general matter, to practice the invention, a nucleic acid according to the invention must be present. It is typically not important to the practice of the method of the invention how the nucleic acid is provided as long as it is suitable for insertion into the selected host cell and subsequent integration into the host cell genome. However, it is preferable that the nucleic acid be of adequate quality and quantity to allow for high efficiency integration. Thus, in some instances, the method comprises providing the nucleic acid of the invention. Providing can be any action that results in the nucleic acid being present in an amount and form that is suitable for introduction of at least one molecule of the nucleic acid into at least one host cell, followed by integration of the molecule into the host cell genome. Preferably, integration is at a pre-selected target site by way of homologous recombination.

While insertion of a nucleotide sequence of interest into a host genome may be a goal of a practitioner, many times the goal will be to express a coding region of interest. Thus, the present invention provides a method of expressing a nucleotide sequence in a host cell. In general, the method comprises: integrating a nucleotide sequence of interest into a host cell genome to create a recombinant cell; and treating the cell with conditions that allow for expression of the nucleotide sequence. The act of integrating can be any act that results in physical integration of the nucleotide sequence of interest into the host cell genome, as discussed above. It is to be noted that the nucleotide sequence of interest may be the entire sequence of a nucleic acid of the present invention, or a portion of it. Treating the cell according to the method can be any act that results in the cell being present in an environment that allows for expression of the integrated sequence of interest. Typically, it comprises incubating (“growing”) the cell in the presence of a growth medium and at a temperature and in an atmosphere that allows for expression of the sequence of interest. Those of skill in the art are well aware of suitable conditions for growth of organisms and expression of proteins, and thus the various conditions need not be detailed here.

While the expressed nucleotide sequence is preferably a nucleotide sequence that encodes a detectable product, and more preferably a product with biological activity, in some embodiments, the nucleotide sequence comprises a sequence that does not encode a gene product. In these situations, the nucleotide sequence is transcribed, but not translated. It can serve as a detectable product, however, by detection of the RNA transcribed from the integrated DNA sequence. However, typically, the method is for producing a peptide, polypeptide, or preferably protein, in a host organism. In general, the method comprises: integrating some or all of a nucleic acid of the invention into the genome of a host organism at a pre-selected target site; and expressing one or more proteins as a result of the integration. Preferably, the integrating occurs by homologous recombination, such as by way of a single crossover homologous recombination event between a target sequence on the host genome and a sequence present on a construct of the invention.

The protein to be expressed may be a protein naturally encoded by the genome of the organism or it may be heterologous to the host cell. Thus, the method may be a method of expressing an endogenous protein in a host cell, such as a fungal cell, or it may be a method of expressing a heterologous protein in a host cell. Expression may be from a transcription control region (e.g., promoter) naturally present in the host genome, which has been operably linked by way of the integration event to a protein coding sequence from the same organism, but which is not naturally linked to the protein coding sequence. Alternatively, expression may be from a control region naturally present in the host genome, which has been operably linked to a heterologous protein coding sequence by way of the integration event. In addition, expression may be from a control region that is heterologous to the host cell genome, and which is operably linked to a host cell gene by way of the integration event. Yet again, expression may be from a heterologous control element linked to a heterologous coding sequence (from the same or different organism as the control region), where both sequences have been introduced into the host genome by way of the integration event. In this latter situation, production of the protein is not only enabled, but the target sequence is interrupted, resulting in abolition of expression of the gene at the target site.

As should be evident from the above disclosure, the present invention provides a protein expression or production platform. The platform comprises a recombinant cell expressing a protein of interest. The platform can be used for production of numerous proteins for various purposes. For example, eukaryotic proteins involved in cellular growth (i.e., metabolism), differentiation, reproduction, pathogenicity, or production of metabolites (e.g., antibiotics or other drugs) can be produced for research or commercial use. For example, proteins can be made for medical or industrial use. Likewise, proteins can be made for further study of certain properties, for example to provide models or insights for engineering of the protein or others like it to improve enzymatic activity, reduce toxicity, improve stability, or any other characteristic of interest. The platform can provide improved efficacy of proteins used for research, therapeutic, or diagnostic applications.

According to the invention, any cell can serve as the cell for the expression platform. However, it is preferred that the cell be a fungal cell, and in particular, a cell of the genus Alternaria, such as A. brassicicola. As shown in the examples, below, A. brassicicola shows high levels of integration efficiency with constructs according to the invention. It likewise is capable of expression of high levels of heterologous proteins.

Exemplary embodiments of the method provide for expression of a heterologous nucleotide sequence that has been inserted into a host genome by homologous recombination of some or all of a nucleic acid of the invention. Other exemplary embodiments provide for expression or overexpression of a gene naturally present in the genome of the host organism, but under the control of a control element that is heterologous to the gene and has been introduced by way of homologous recombination of at least part of a nucleic acid of the invention. Yet other exemplary embodiments provide methods of expressing a protein while concurrently reducing or eliminating (e.g., knocking-out or silencing) the expression of a host gene in a host genome by inserting a nucleic acid of the invention into the expression control region or coding region of the gene. Thus, the invention provides, within the context of the expression platform as well as the context of other aspects of the method, a method of altering the expression of a host gene in a host genome.

In yet a further aspect, the invention provides methods for identifying genes and proteins having a detectable effect on cells. In general, the method comprises: generating a recombinant cell according to the invention having at least one characteristic of interest; and detecting the characteristic(s). According to the method, generating a recombinant cell may be any of the various actions discussed above. Preferably, the recombinant cell will be one that has a gene that has been specifically altered by integration of a nucleic acid of the invention. In highly preferred embodiments, the gene has been altered by homologous recombination between sequences of the gene and sequence of a nucleic acid of the invention.

The act of detecting the characteristic can be any act that allows one to determine whether a selected characteristic is present or absence in the recombinant cell. For example, where the characteristic is growth in the presence of a toxic compound, the recombinant cell can be detected by determining if it can grow in the presence of the toxic compound. Where the characteristic is presence being essential for reproduction, the act of detecting can be through exposing the cell to conditions that allow for reproduction, and determining if the cell was capable of doing so. As an additional non-limiting example, where the characteristic is enzymatic activity, the act of detecting can be exposing the cell, cell lysate, or a purification fraction of the lysate, to reaction conditions that are suitable for assaying the enzymatic activity, and determining if the cell, lysate, or fraction has the activity. Detecting can thus be any act, including but not limited to, detection of growth, survival, reproduction, or pathogenicity; detection of expression of a detectable nucleic acid sequence; detection of expression of a peptide, polypeptide, or protein, such as an enzyme, an immunoreactive peptide, or a polypeptide; detection of production of a toxin, a growth regulator, or a small molecule bioactive agent (e.g., antibiotic, small molecule second messenger).

For example, the method can be a method of high-throughput screening for genes having certain effects on the growth, maintenance, and/or environmental activity of cells. In embodiments, the invention provides methods of high-throughput screening for genes and gene products involved in pathogenesis of fungi, such as pathogenesis of fungal plant pathogens. Among other things, it also provides methods for screening for genes and gene products that have toxic or other effects on other organisms, such as on plants. The method of high-throughput screening can be practiced as follows: a series (library) of LMEs according to the invention are created, each having the same detectable characteristic (e.g., antibiotic resistance) but each having a sequence from the host cell genome that is different; a collection (e.g., culture) of host organism cells are exposed to the library under conditions where the LMEs can integrate into the genomes of the host cells to create recombinant cells; the recombinant cells are identified by assaying for the detectable characteristic; and the recombinant cells are assayed for one or more other detectable characteristics, the presence or absence of which indicates alteration of a gene involved in expression of that characteristic. The sequences from the host genome, which serve as sites for homologous recombination with the host cell genome, can be engineered based on known sequences of the host genome, can be engineered based on genomic sequences of closely related species, or can be randomly generated.

The method of high-throughput screening is rendered feasible by the present invention due to the exceptionally high rate of transformation seen using the nucleic acids of the invention. That is, because transformation efficiencies reproducibly approach 100%, a large number of different transformants may be created in a short period of time, and a collection, or library, of different recombinant cells can be prepared and screened for one or more characteristics.

It is to be understood that all of the methods discussed herein can comprise one or more control reactions to ensure that the various actions of the methods and protocols are performing as expected. Thus, any number of positive or negative control reactions may be employed as part of the methods of the invention. For example, where a method includes detecting growth in the presence of a toxic substance, a control reaction comprising growth of a wild-type organism, which is identical to the recombinant cell of the invention except for integration of the nucleic acid of the invention in the recombinant cell's genome, can be performed to ensure that the wild-type cell does not grow, thus confirming that the substance is present in toxic amounts. Other controls, such as, but not limited to, those known in the art for determining proper performance of molecular biology techniques, enzymatic assay techniques, and cellular growth, pathogenicity, or reproduction can be included in the methods.

In yet another aspect, the invention provides kits. In general, the kits comprise some or all of the nucleic acids, cells, compositions, and reagents needed to perform at least one method of the invention. In general, kits according to the invention will comprise at least one nucleic acid of the invention. Typically, kits comprise one or more containers, which independently contain a nucleic acid, a composition, a cell, or a reagent useful in a method of the invention. Kits are typically made of a shell comprising cardboard, plastic, or other convenient material for storing and protecting the containers and other components of the kit. The shell contains one or more containers, along with optional additional components, such as supplies and materials (e.g., growth media, antibiotics, enzyme substrates) for practice of a method.

### EXAMPLES

The invention will be further explained by the following Examples, which are intended to be purely exemplary of the invention, and should not be considered as limiting the invention in any way.

### Example 1

### Generation of Mutants with a Linear Minimal Element

Alternaria brassicicola causes black spot disease of cultivated Brassicas and has been used consistently as a necrotrophic fungal pathogen for studies with Arabidopsis. In A. brassicicola, mutant generation has been the most rate-limiting step for the functional analysis of individual genes due to low efficiency of both transformation and targeted integration. To improve the targeted gene disruption efficiency as well as to expedite gene disruption construct production, we used a short linear construct with minimal elements, an antibiotic resistance selectable marker gene, and a 250- to 600-bp-long partial target gene. The linear minimal element (LME) constructs consistently produced stable transformants for diverse categories of genes. Typically, 100% of the transformants were targeted gene disruption mutants when using the LME constructs, compared with inconsistent transformation and usually less than 10% targeted gene disruption with circular plasmid disruption constructs. Each mutant displayed a unique molecular signature thought to originate from endogenous exonuclease activities in fungal cells. The data presented in this Example suggests that a DNA double-stranded break repair mechanism (DSBR) functions to increase targeting efficiency. This method is advantageous for high throughput gene disruption, overexpression, and reporter gene introduction within target genes, especially for asexual filamentous fungi, where genetic approaches are unfavorable.

In the present Example, we used a relatively easy and economical PEG-mediated protoplast transformation method to disrupt the function of individual genes by targeted gene disruption with an unconventional linear construct composed of an antibiotic resistance selectable marker gene at one side and a partial target gene sequence at the other side. The utility of this approach for high throughput gene disruption and overexpression of target genes in A. brassicicola and other organisms is evident from the data.

Materials and Methods Used in this Example:

Fungi: unless otherwise noted, A. brassicicola isolates ATCC34622, ATCC96866, and AB7 were used in this Example. ATCC96866 is the isolate currently being sequenced. A. brassicicola was routinely cultured on PDA media (Difco, Kansas City, Mo., U.S.A.). A. alternata isolate ATCC 11680 was used in this Example.

Generation of disruption constructs: Based on the cDNA sequence, two primers with an enzymatic site at each end were designed to amplify a 250- to 500-bp fragment within the coding region of each gene. PCR products were digested with the two endonucleases and ligated into the multiple cloning site of pCB 1636 plasmid. The plasmid construct was transformed in Escherichia coli DH5cs (Invitrogen, Carlsbad, Calif., U.S.A.) to produce over 10 ug of plasmid. The plasmid constructs were sequenced to verify the presence of target gene sequences in the vector. These constructs were used in either transformation experiments in their circular form or in PCR reactions to generate linear DNA. Constructs were used as template DNA to amplify between M13 forward and M13 reverse priming sites that contained the hygB phosphotransferase gene under control of the trpC fungal promoter and the cloned partial targeted gene. The PCR products were purified with the PCR Clean Up Kit (Qiagen, Palo Alto, Calif., U.S.A.) and further concentrated to a concentration of 1 ug/ul in a Speedvac (Eppendorf, Barkhausenweg, Germany).

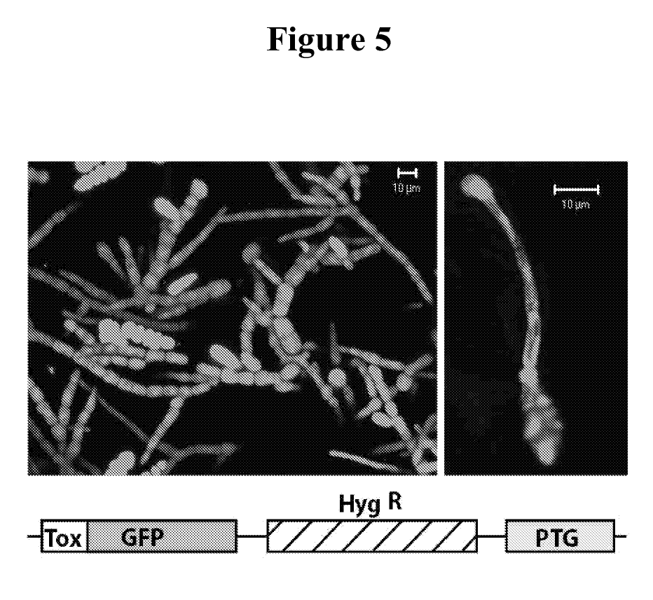

GFP-tagged construct and overexpression constructs: To make GFP-tagged LME disruption constructs, we added GFP cassettes in front of hygB resistance cassettes (see, for example, FIG. 5). The GFP cassettes containing ToxA promoter and a GFP coding gene were amplified with primers 5′-acggggtaccTTGGAATGCATGGAGGAGT-3′ (SEQ ID NO:1) and 5′-ttatctcgagTTGCGCGCTATATTTTGTTTT-3′ (SEQ ID NO:2) using pCT74 as template DNA. The PCR products were digested with XhoI and KpnI and cloned in pCB-Nac to make plasmid pCB1606-Nac. Cloning and plasmid production were performed in accordance with routine procedures. A fragment containing green fluorescent protein (GFP) cassettes, Hyg cassettes, and partial N-acetylglucosaminidase (GFP-tagged LME disruption constructs) was amplified using pCB 1606-Nac as template DNA with M13 forward and M13 reverse primers. The PCR products were transformed in wild-type A. brassicicola to make GFP-tagged N-acetylglucosaminidase disruption mutants.

An LME overexpression cassette for N-acetylglucosaminidase was made by cloning Nourseothricin-resistant cassettes, two constitutive promoters, and partial target gene in pBluscript II SK(−), at SpeI and PstI sites, HindIII, and XbaI sites, and ApaI and XhoI sites, respectively. Nourseothricin cassettes were amplified with primers 5′-GTGCACTAGTTCATTCTAGCTTGCGGTCCT-3′ (SEQ ID NO:3) and 5′-ACATCCACGGGACTTGAGAC-3′ (SEQ ID NO:4) using pNR as template DNA. ToxA and TrpC promoters were amplified with primers 5′-GGCTCTCGAGTTTGGATGCTTGGGTAGATAG-3′ (SEQ ID NO:5) and 5′-TTGCTAAGCTTGGCTATATTCATTCATTGTCAGC-3′ (SEQ ID NO:6) using pCT74 as template DNA. Partial sequence of N-acetylglucosaminidase was amplified with primers 5′-ACAACTCGAGCAGCAATGCGCGATTTCATA-3′ (SEQ ID NO:7) and 5′-AGGTGGGCCCTACGCCGTCTGGTTCAAATAC-3′ (SEQ ID NO:8) using A. brassicicola genomic DNA from the start codon.

A. brassicicola transformation: Transformation was carried out with either plasmid disruption constructs or linear PCR products based on the transformation protocol of A. alternata (Akamatsu et al. 1997), with modifications. Approximately 5×10⁶ fungal conidia were harvested from a PDA culture plate and inoculated into 50 ml of GYEB (1% glucose and 0.5% yeast extract) media. They were cultured for 36 hours with shaking at 100 rpm at 25° C. The mycelia were harvested by centrifugation at 2,000×g for 5 minutes and washed with 0.7 M NaCl followed by centrifugation again under the same conditions as before. The mycelia were digested in 6 ml of Kitalase (Wako Chemicals, Richmond, Va., U.S.A.) at 10 mg/ml in 0.7 M NaCl for 3 to 4 hours at 28° C. with constant shaking at 110 rpm. The protoplasts were collected by centrifugation at 700×g for 10 minutes at 4° C., washed twice with 10 ml of 0.7 M NaCl, and then with 10 ml of STC buffer (1 M Sorbitol, 50 mM Tris-HCl, pH 8.0, and 50 mM CaCl₂). The protoplasts were resuspended in STC at a concentration of 4×10⁶ in 70 ul, after which 10 ug of plasmid or PCR products in 10 ul of ddH2O was added to the protoplast and gently mixed. The transformation mix was incubated on ice for 30 minutes. Heat shock transformation was performed by incubating the transformation mixture at 42° C. for 2 to 10 minutes. The transformation mix was incubated at room temperature after the addition of 800 ul of 40% PEG solution. Then, 200 ul of the transformation mixture was added to 25 ml of molten regeneration medium (1 M sucrose, 0.5% yeast extract, 0.5% casein amino acids, and 1% agar) in a 50 ml tube and subsequently poured into a 100-by-15-mm petri dish. After 24 hours, the plates were overlaid with 25 ml of hygB (Sigma-Aldrich, St. Louis, Mo., U.S.A.) containing PDA at 30 ug/ml. Individual hygB resistant transformants were transferred to a fresh hygB-containing plate between 10 and 15 days after each transformation. Each transformant was purified further by transferring a single spore to a fresh hygB-containing plate.

DNA Isolation: Total genomic DNA from A. brassicicola was extracted using a phenol-chloroform extraction method. Fungi were grown for 2 to 3 days in 50 ml of GYEB media. Approximately 0.2 g of mycelia was harvested and filtered with Miracloth (Calbiochem, Darnstadt, Germany), semi-dried with paper towels, and ground into fine powder with a mortar and pestle in the presence of liquid nitrogen. The samples were mixed with 1 ml of extraction buffer (0.7 M NaCl, 1% cetyltrimethylammonium bromide, 50 mM Tris-HCl, pH8.0, and 10 mM EDTA) and 10 ul of RNAse A (Sigma-Aldrich) at 100 mg/ml and incubated at 60° C. for 30 minutes. Water (1 ml) was added to the tube and extracted with 2 ml of Tris-saturated phenol, followed by chloroform extraction. DNA was separated from the solution by adding an equal volume of cold ethanol to the upper phase. DNA was transferred with a plastic stick to a clean tube and washed with 70% ethanol.

Mutant Verification with PCR: The genomic DNA was used for PCR verification of the disruption mutants with a gene-specific primer and a hygB gene-specific primer, or two gene-specific primers outside of the disruption construct. A total of 30 ng of genomic DNA was used in each 20 ul PCR reaction in a GeneAmp PCR system 2700 thermocycler (ABI, Foster City, Calif., U.S.A.). With the gene-specific and hygB-specific primers, PCR reactions were performed with Taq polymerase to amplify approximately 2.5 kb by 94° C. denaturation for 5 minutes followed by 30 cycles of 30 seconds of denaturation at 94° C., 30 seconds of annealing at 55° C., and 2 minutes of elongation at 72° C. After the cycling reactions, the final elongation was performed at 72° C. for 7 minutes. With gene-specific primers only, PCR reactions were performed with AccuPrime Taq (Invitrogen) by 94° C. denaturation for 1.5 minutes followed by 30 cycles of 10 seconds of denaturation at 94° C., 10 seconds of annealing at 55° C., and 3.3 minutes of elongation at 68° C. PCR products were size fractionated on 1% agarose gel by electrophoresis and visualized by ethidium bromide staining.

Southern Hybridization Analysis: A total of 2 to 3 ug of genomic DNA was digested with selected enzymes purchased from New England BioLabs (Beverly, Mass., U.S.A.). The digested DNA was size fractionated on a 0.7% agarose gel, followed by transfer to Hybond Nylon membrane (Amersham, Piscataway, N.J., U.S.A.). DNA probes were synthesized using a PCR digoxigenin (DIG) Probe Synthesis Kit according to the manufacturer's manual (Roche Diagnostics, Mannheim, Germany). Hybridization of membranes was performed at 50° C. using the Block and Wash Buffer set according to the manufacturer's instructions (Roche Diagnostics). Membranes were washed in a final solution of 0.1×SSC (1×SSC is 0.15 M NaCl plus 0.015 M sodium citrate) and 0.1% sodium dodecyl sulfate at 68° C. Chemiluminescent detection was used to develop the blots with CDP-Star using the DIG detection kit according to the manufacturer's instructions (Roche Diagnostics).

Quantitative Real-Time PCR: RNA was extracted from 0.1 g of mycelia grown in GYEB media using the Plant RNeasy kit (Qiagen). RNA samples were checked for quality on the Agilent Bioanalyzer 2100 (Agilent, Palo Alto, Calif., U.S.A.). Then, 1 ug of total RNA was transcribed to cDNA in 20 ul using the BioRad I-script kit. Each cDNA was diluted 1:10, with 1 ul (5 ng of total RNA) used per reaction. Reactions consisted of 300 nM sense and antisense primers, 1 ul of diluted cDNA, and reverse transcriptase-grade PCR water to a final volume of 12.5 ul. SYBR-Green Supermix (BioRad, Hercules, Calif., U.S.A.) was added to obtain a final running volume of 25 ul per reaction. Each reaction was run in triplicate for both the standard and unknown samples. Reactions were run under the following conditions in the Bio Rad 1-cycler (BioRad): 95° C. denaturation for 3 minutes, 40 cycles of 95° C. for 10 seconds, 56° C. for 15 seconds, and 72° C. for 20 seconds to calculate cycle threshold values, followed by 95° C. for 1 minute, 55° C. for 1 minute, and 80 times of 55° C. for 10 seconds, increasing temperature by 0.5° C. each cycle to obtain melt curves and, subsequently, to enable data analyses. Standard curves were produced with purified amplified DNA products of 10 and 1 pg/ul and starting concentrations of 100, 10, and 1 fg/ul. A baseline subtracted curve fit was used to generate standard curve data. Absolute amounts of transcripts were calculated using a correlation coefficient formula generated from the standard curve in each run without length correction of 1.8-kb actual transcripts compared with 200-bp amplicons.

PEG-Mediated Transformation of A. brassicicola Protoplasts With Circular Plasmids: Prior to initiating transformation experiments, we performed a hygB sensitivity assay and found that hygB in potato dextrose agar (PDA) media at 15 ug/ml was sufficient to abolish growth of A. brassicicola using several isolates of the fungus. Initial transformation experiments (PEG-mediated transformation of protoplasts) were performed using one isolate (ATCC34622) with various circular fungal transformation vectors, including pCB 1636, pCB 1004, pCT74, and pAN7-l (Lorang et al. 2001; Punt et al. 1987; Sweigard et al. 1997). In some experiments, vectors were linearized by cutting with an appropriate restriction enzyme prior to transformation. Regardless of vector used, transformation efficiency using circular plasmids was extremely low, with typically less than three hygB-resistant transformants generated per microgram of plasmid when using between 10⁶ and 10⁷ protoplasts (Table 1).

TABLE 1
Transformation Frequencies Of A. brassicicola
Using Diverse Plasmid Vectors

| | No. of | A. brassicicola | Trans/10⁷ | Mutants/ |
|---|---|---|---|---|
| Plasmid^{a} | Expts. | Isolates | Protoplasts^{b} | trans^{c} |
| pCB 1004 | | | | |
| uncut | 2 | ATCC34622 | 0, 0 | |
| HindIII | 2 | ATCC34622 | 0, 0 | |
| pAN 7-1 | | | | |
| uncut | 3 | ATCC34622 | 0, 0, 0 | |
| HindIII | 3 | ATCC34622 | 0, 2, 4 | |
| pCT74 | | | | |
| uncut | 2 | ATCC34622 | 2, 1 | |
| pCB 1636 | | | | |
| uncut | 3 | ATCC34622 | 0, 3, 0 | |
| HindIII | 3 | ATCC34622 | 0, 5, 3 | |
| EcoRI | 3 | ATCC34622 | 6, 7, 7 | |
| pCB-MFS* | 1 | ATCC34622 | 8 | 5/8 |
| PCB-CPS* | 1 | ATCC34622 | 20 | 1/20 |
| pCB-CYH* | 1 | ATCC34622 | 32 | 2/32 |
| pCB-Altb1* | 1 | ATCC96866 | 20 | 0/20 |
| pCB-MAPK* | 6 | ATCC96866 | 17** | 4/17 |
| pCB-LIP1* | 1 | ATCC96866 | 7 | ND |
| pCB-LIP2* | 3 | ATCC96866 | 20** | 1/3 |
| pCB-LIP3* | 2 | ATCC96866 | 7*, 5 | 3/4 |

^{a}Asterisk (*) indicates pCB 1636 with partial A. brassicicola gene sequence;
^{b}Number of transformants/10⁷ protoplasts; Asterisks (**) indicates total numbers of transformants from multiple experiments;
^{c}Number of knock-out mutants/transformants examined. ND indicates not determined.

In general, linearization of these circular plasmids prior to transformation increased transformation efficiency only slightly. In contrast, transformation efficiency was improved dramatically, up to 10-fold in several cases, with circular constructs using the pCB 1636 vector harboring partial A. brassicicola gene sequences. Another isolate (ATCC96866) showed moderately high transformation efficiency with similar type constructs. However, targeting efficiency typically was less than 10% in both strains; thus, this approach was not suitable for high-throughput functional analysis of genes (Table 1). To potentially increase transformation and gene disruption efficiency, we elected to use a shorter, linear, double-stranded polymerase chain reaction (PCR) product with minimal elements, including a hygB resistance gene cassette and the various target gene fragments in subsequent transformation attempts in lieu of using the entire circularized construct.

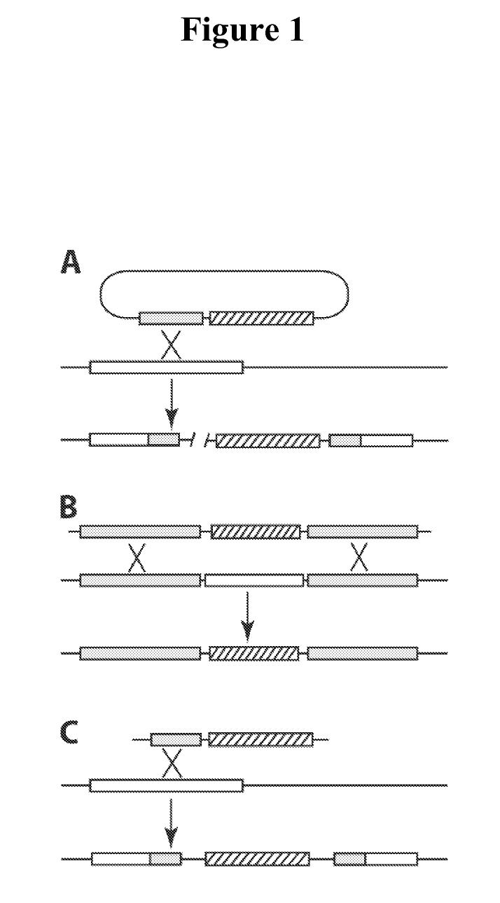

Linear Minimal Element Constructs—New Logic For Fungal Gene Disruption: There are several established methods to disrupt target genes using diverse constructs. A representative circular disruption construct contains an internal fragment of a target gene cloned together with a selectable marker gene in a plasmid vector (see, for example FIG. 1A). A classic linear construct, represented by the replacement construct, flanks an antibiotic resistance or other appropriate selectable marker genes with two fragments representing 5′ and 3′ regions of a target gene (see, for example, FIG. 1B). Derivatives of replacement constructs have been widely used in diverse filamentous fungi to either replace short segments within a target gene with a selectable marker gene or disrupt a target gene (Bussink and Osmani 1999; Shah-Mahoney et al. 1997; Shiotani and Tsuge 1995; Voigt et al. 2005). We generated a linear construct with a partial target gene at only one end and an antibiotic resistance selectable marker gene at the other end (see FIG. 1C), and designated this as a linear minimal element (LME) disruption construct to distinguish from other types of linear constructs. More specifically, FIG. 1 shows a diagram depicting incorporation of transforming DNA at a target genomic locus. Shown are targeted gene disruption or replacement mechanisms starting with A, a circular construct, B, a linear recombinational gene replacement construct, and C, a linear minimal element (LME) construct. Hatched box=a selectable marker gene, white box=a target genomic locus (A, B, and C) and gray box=partial target gene (A and C) or target flanking regions (B). Homologous recombination events are shown with a large “X” between target genomic locus and a partial target gene (A and C) or between target gene flanking regions (B).

Insertion of classic linear constructs by a single homologous recombination was previously reported as an undesirable side effect in several cases due to the circularization of the linear constructs after transformation (Bussink and Osmani 1999; Shah-Mahoney et al. 1997; Yang et al. 2004). In contrast, we have found that, if the LME construct is circularized efficiently in the cell, gene disruption can occur effectively, for example by a single homologous recombination event (FIG. 1C).

Transformation And Targeted Gene Disruption Efficiency Using LME Constructs: We created over 20 individual disruption constructs, using the pCB 1636 vector harboring partial cDNA sequences corresponding to A. brassicicola genes identified in various cDNA libraries from our laboratory, to ultimately evaluate their role in pathogenicity (see Table 2). We produced LME disruption constructs by PCR-based amplification of the gene-specific fragments and the hygB resistance gene cassette with M13 forward and M13 reverse primers using plasmid constructs as template DNA.

To establish a new gene disruption method, we transformed the LME constructs primarily into an A. brassicicola isolate (ATCC96866) and several other isolates. Importantly, the whole genome draft sequence currently is being determined for the ATCC 96866 isolate. We tested LME constructs to disrupt genes encoding putative toxic proteins, hydrolytic enzymes, a signaling pathway component, a transcription factor, and an essential protein (Table 2). Numbers of transformants generated in these experiments ranged from 3 to 40 that emerged within 2 weeks on the selection plate for each gene except an essential gene (Table 2). Additional transformants surfaced after 16 days. Late-emerging transformants were not counted in this study because previously emerged colonies grew too large and overlapped the late-emerging colonies. On average, 15 transformants emerged within 16 days when we transformed 10 ug of LME disruption constructs in 4×10⁶ protoplasts with an optimized protocol.

We obtained 100% gene disruption efficiency according to PCR and Southern hybridization experiments in most of these experiments (see next section). In addition, no evidence of ectopic insertion was observed in the vast majority of targeted gene disruption mutants. One or two rounds of single-spore isolation on PDA media containing hygB were sufficient to purify mutants and eliminate contamination with wild-type nuclei. After the protocol was optimized, we transformed two additional isolates (ATCC36422 and AB7) to find comparably high (100%) targeted gene disruption efficiency for three genes (Table 2). In addition, we encountered extremely high transformation efficiency using the AB7 isolate.

We qualitatively assessed stability of gene disruption mutants after one round of single-spore isolation with isolate ATCC96866. Three different hygB-resistant mutants for genes predicted to encode an fus3 MAP kinase, chymotrypsin, and N-aceTylglucosaminidase were cultured on PDA media lacking hygB for approximately 1 week, and hyphal tips were repeatedly transferred on to new PDA plates. The repeated subculturing on PDA media did not change hygB resistance of the disruption mutants during the repeated hyphal tip transfers, even after five times. After isolation of new conidia produced during late stages of plant infection, we also tested growth ability of mutant conidia on hygB-containing PDA media several times with over 10 different mutants. In every case, recovered spores remained highly resistant to hygB at the levels used in our studies. Thus, we strongly believe that this transformation method produces highly stable, hygB-resistant transformants.

TABLE 2

| | | Number Of | Knock-out | | |
|---|---|---|---|---|---|
| | | Transformants | percentage | | |
| | | Per 1 × 10⁶⁻⁷ | (no. of mutants/ | | Verification |
| Gene^{a} | Strain | Protoplasts | no. examined)^{b} | | Method |
| Fus3 MAP Kinase | ATCC96866 | 20 | 100 | (20/20) | Morphology/PCR |
| Fus3 MAP Kinase | AB7 | 5 | 100 | (5/5) | Southern |
| Fus3 MAP Kinase | ATCC34622 | 1 | 100 | (1/1) | Morphology |
| Hog MAP Kinase | ATCC96866 | 13 | ND | | Morphology |
| Chymotrypsin | ATCC96866 | 10 | 80 | (4/5) | Southern, PCR |
| N-acetylglucosaminidase | ATCC96866 | 12 | 100 | (6/6) | Southern, PCR |
| N-acetylglucosaminidase | AB7 | 120 | 100 | (5/5) | PCR |
| N-acetylglucosaminidase | ATCC34622 | 7 | ND | | |
| Glycosyl hydrolase | ATCC96866 | 15 | ND | | |
| Pectate lyase | ATCC96866 | 10 | ND | | |
| Pectate lyase | AB7 | 64 | 100 | (5/5) | PCR |
| Pectate lyase | ATCC34622 | 9 | 100 | (5/5) | PCR |
| Zinc finger | ATCC96866 | 31 | 100 | (8/8) | Southern, PCR |
| Polyketide synthase | ATCC96866 | 3 | 100 | (3/3) | Southern, PCR |
| Altb1 Allergen | ATCC96866 | 4 | 100 | (4/4) | Southern, PCR |
| ATP/ADP transporter | ATCC96866 | >200 | 98 | (196/200) | Viability |
| Lipase1-1 | ATCC96866 | >10 | 100 | (5/5) | PCR |
| Lipase1 | ATCC96866 | 8 | 100 | (8/8) | Southern |
| Lipase2 | ATCC96866 | 15 | 100 | (6/6) | Southern |
| Lipase3 | ATCC96866 | 14 | 93 | (13/14) | Southern |
| Cutinase (CL394) | ATCC96866 | >10 | 100 | (6/6) | Southern |
| N-ace overexpression(1) | ATCC96866 | >10 | | | QRT-PCR |
| N-ace overexpression(2) | ATCC96866 | 34 | | | ND |
| N-ace overexpression(3) | ATCC11680 | 15 | | | QRT-PCT |
| N-ace KO + GFP tag | ATCC96866 | >10 | 100 | (1/1) | PCR |

^{a}N-ace over (1) = overexpression of N-acetylglucosaminidase in wilde type, N-ace(2) = overexpression in Fus3 MAP kinase mutant, and N-ace(3) = overexpression in A. alternata wild type

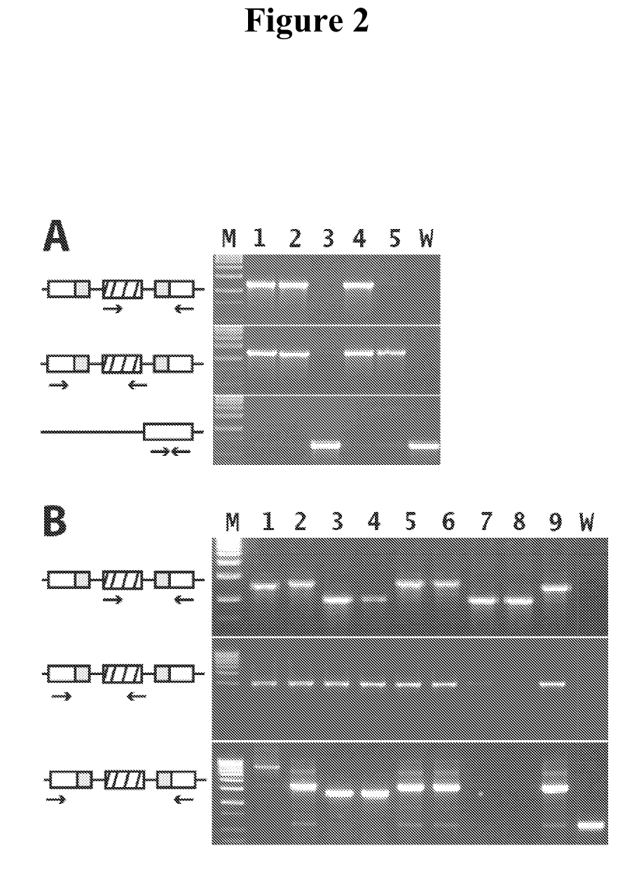

Mode Of Targeted Gene Disruption Using LME Constructs: Here, we describe molecular data with two genes predicted to encode chymotrypsin and N-acetylglucosaminidase to support efficiency and mode of gene disruption. For the gene predicted to encode a chymotrypsin-like enzyme, we randomly examined 5 of 10 hygB-resistant transformants generated with an LME disruption construct harboring a partial gene sequence. Following one round of single-spore isolation, we found that four of them were targeted gene disruption mutants, whereas one maintained the target gene intact based on PCR examination (FIG. 2A). Thus, the transforming DNA in this case was inserted ectopically. In three of four chymotrypsin gene disruption mutants, there were two incomplete gene sequences flanking the hygB resistance cassette. Template genomic DNA isolated from one of the four disruption mutants generated PCR products from only one primer pair (FIG. 2A, lane 5), suggesting molecular differences from other mutants (examples shown below). More specifically, FIG. 2 shows verification of targeted gene disruption by polymerase chain reaction (PCR) for the chymotypsin gene (Panel A), and the N-acetylglucosaminidase gene (Panel B). PCR amplification was performed with two pairs of target gene-specific and hygB resistance gene primers (top two panels) and a pair of solely gene-specific primers (bottom panel). Relative primer binding locations are marked below the schematic diagram of the disrupted target gene on the left side of gel images. The diagrams were drawn with the understanding that the target gene was disrupted via single homologous recombination with a circularized linear minimal element (LME) construct. Band sizes corresponding to three mutants (ace2, aceS, and ace6) were similar to the expected ones based on a single copy insertion of an LME construct. There was variation in PCR product size corresponding to other mutants. In the figure, the hatched box=a selectable marker gene, the white box=a target genomic locus, and the gray box=partial target gene. Weak bands on the third panel for the N-acetylglucosaminidase gene are due to minor contamination of wild-type nuclei or heterokaryons. These were undetected by Southern hybridizations even after an extreme exposure. Amplification was performed with Taq polymerase for PCR reactions shown in the first five panels (top to bottom) and with Accuprime Taq (Invitrogen, Carlsbad, Calif., U.S.A.) for reactions depicted in the last panel due to long (over 3 kb) expected product size. M=1-kb molecular marker (NEB, Beverly, Mass., U.S.A.).

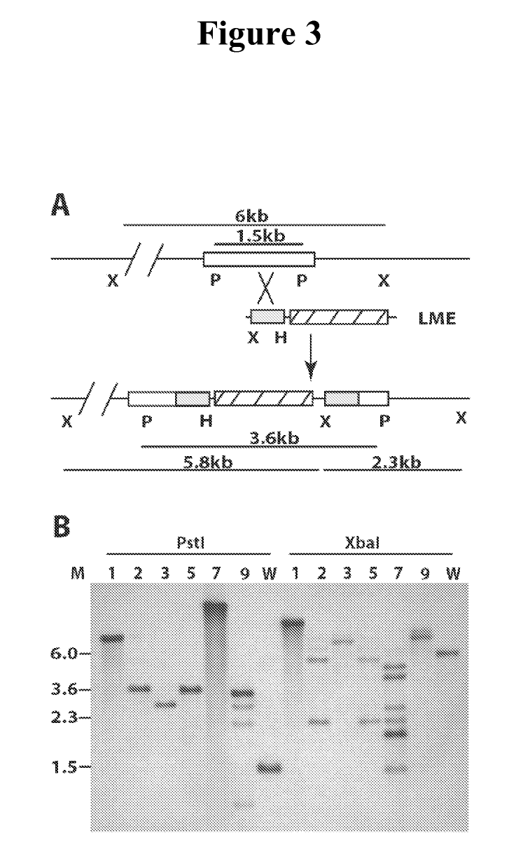

We examined nine randomly selected hygB-resistant transformants generated with an appropriately designed LME disruption construct and following single spore isolation for another gene predicted to encode N-acetylglucosaminidase. Interestingly, disruption mutants generated with this construct showed at least four PCR amplification patterns (FIG. 2B). Three of nine transformants (designated ace2, aceS, and ace6) produced PCR products (FIG. 2B) and Southern hybridization patterns that would be expected from a mutant with a single-copy insertion of an LME construct in the target gene (FIG. 3). The other six transformants also produced PCR products and Southern hybridization results expected from targeted gene disruption mutants in principle, but they presented unusual features. For example, PCR and Southern hybridization patterns generated from ace1 and ace7 genomic DNA indicated 3 and >10 copies of insertion of the LME constructs in the target gene, respectively. Interestingly, three transformants (ace3, ace4, and ace9) showed smaller amplification products in PCR experiments than the first three single-insertion mutants (ace2, aceS, and ace6) (FIG. 2B).

Southern blotting experiments revealed more complex hybridization patterns using genomic DNA isolated from ace3, ace4, and ace9 mutants compared with ace2, aceS, and ace6 (FIG. 3B). FIG. 3 shows a restriction map and Southern blot analysis of N-acetylglucosaminidase gene mutants. Panel A shows a restriction map of the N-acetylglucosaminidase genomic locus, the linear minimal element (LME) disruption construct, consisting of a 600-bp partial gene fragment (gray) and 1.5-kb hygB resistance gene cassette (hatched box), and a typical gene disruption mutant locus. The expected disruption mutant genomic locus diagram is based on a single homologous recombination occurring between the target gene and a circularized LME construct. P=PstI, X=XbaI, and H=HindIII. Panel B shows Southern hybridization of genomic DNA from N-acetylglucosaminidase disruption mutants (1 through 9) and wild type (w). Each DNA set was digested with PsI or XbaI. The blot was probed with digoxigenin-labeled partial target sequence contained in the disruption construct (gray box). All disruption mutants showed variation in band size compared with the wild type. Two mutants (ace2 and aceS) showed the banding patterns and sizes expected based on the schematic diagram (A). Loss of an XbaI site is apparent for ace 1, ace3, and ace9. Origin of relatively faint bands corresponding to ace9 was not able to be determined. Estimated size of DNA fragments were marked on left and based on 1-kb ladder (Fisher Scientific, Atlanta, Ga., U.S.A.).

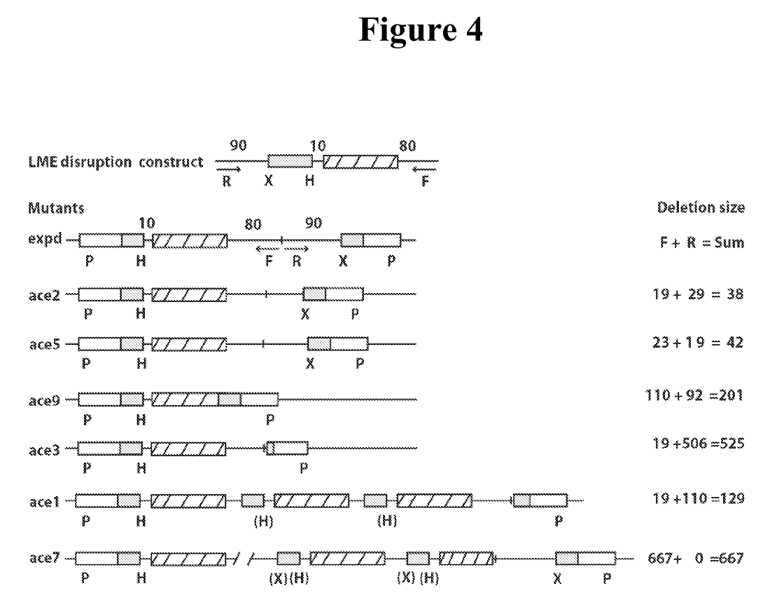

We next determined DNA sequences of PCR products at the target gene locus (FIG. 2B) amplified from genomic DNA extracted from the individual mutants to verify arrangement and sequence length of the target gene and the LME construct. These PCR products were amplified using two pairs of gene specific primers; thus, hygB resistance-gene-specific primers would amplify the genomic locus harboring the LME construct. DNA sequencing of PCR products revealed typical arrangement of a genomic locus that has undergone a single homologous recombination with a circular disruption plasmid (FIG. 4). More specifically, FIG. 4 depicts a schematic diagram of targeted gene disruption with linear minimal element (LME) construct. The vector fragment at both ends of the initial construct is marked with F and R (M13 forward and reverse priming sites) with sequence directions (arrows). The length (bp) of vector sequence is shown at three locations (90, 10, and 80). The symbol (expd) represents targeted gene disruption by a single homologous recombination between a circularized LME construct and the target gene, whereas others show gene organization of actual mutants based on three lines of evidence: polymerase chain reaction (PCR) product size, Southern hybridization banding patterns, and sequence verification of PCR products for all except ace9. The ace9 diagram is based solely on PCR product size and sequence information. A short vertical line in the diagram of mutants is a boundary between both ends of vector fragment. The distance between the vertical line and adjacent boxes represents the length of nucleotides present. The extent (base pairs) of nucleotide deletion at M13F and M13R sides is depicted on the far right. The symbol ( ) indicates probable presence of restriction enzyme site that was not experimentally tested. Abbreviations: H=HindIII, X=XbaI, and P=PstI.

Noticeably, the M13F and M13R side vector sequences existed facing away from each other in mutants. Collectively, these observations strongly suggested that the target gene disruption occurred by homologous recombination with a circularized LME construct. Consistent with the PCR and Southern hybridization results, length variation among mutants solely originated from deletions at the ends of LME constructs (FIG. 4). Sequence lengths in all mutants were shorter than the expected size, ranging from 38- to 667-bp deletions. Based on characterization of the 14 transformants, it is apparent that targeted gene disruption by homologous recombination is a predominant mechanism over ectopic insertion of the LME constructs. Finally, endogenous exonuclease digestion and circularization of LME constructs appears to precede the homologous recombination.

Application Of LME Constructs Using Green Fluorescent Protein Tagging Of Mutants And Targeted Gene Overexpression Mutants: The LME disruption constructs consistently produced targeted gene disruption mutants for various genes with close to 100% efficiency. We used the same method to tag the mutants with green fluorescent protein (GFP) gene simultaneously during the targeted gene disruption process. We added a 1,345-bp-long GFP expression cassette in front of the N-acetylglucosaminidase disruption constructs (FIG. 5, diagram). More specifically, FIG. 5 shows green fluorescent protein (GFP)-tagged N-acetylglucosaminidase gene disruption mutants. The left panel shows GFP expression of a mutant growing on hygromycin containing potato dextrose agar, while the right panel shows an appressorium-like structure at the hyphal tip of a germinated spore on a Brassica oleracea (green cabbage) leaf surface 12 hours following inoculation. Abbreviations: Tox=ToxA promoter and PTG=partial target gene.

Over 10 transformants were produced in approximately 11 days. Because we knew the targeted gene disruption efficiency was close to 100%, we examined two of the early emerging transformants to find both targeted gene disruption mutants (data not shown). The GFP was expressed constitutively due to the ToxA promoter at the 5′ side of the GFP coding gene. The mutants were subjected to visualization with a fluorescence microscope. N-acetylglucosaminidase mutants made the typical appressoria like structure during plant infection (FIG. 5, right), but not during saprophytic growth on a PDA plate (FIG. 5, left).

Furthermore, we tested a novel possibility of using the LME construct approach to overexpress target genes. We made new LME constructs for the N-acetylglucosaminidase gene with a new antibiotic-resistant selectable marker gene (nourseothricin acetyltransferase) under control of the OliC promoter. In addition to this cassette, constructs contained two constitutive promoters (TrpC and ToxA) followed by the start codon of the N-acetylglucosaminidase gene (FIG. 6, diagram). More specifically, FIG. 6 shows overexpression of the N-acetylglucosaminidase gene measured by quantitative reverse-transcriptase polymerase chain reaction. Panel A: shown is a schematic diagram of a linear minimal element (LME) overexpression construct. Antibiotic resistance marker gene cassettes (OliC promoter and nourseothricin acetyltransferase gene) precede the N-acetylglucosaminidase gene with TrpC and Thx A promoter sequences. Gene expression level is presented in Panels B and C: six transformants in Alternaria brassicicola are shown in (B), while eight transformants in an A. alternata wild-type background are shown in (C). Sample RNA was extracted from mycelia grown in glucose-yeast extract (GYEB) media for 4 hours. The X-axes show sample identification and Y-axes show absolute amounts of transcripts (femtogram) in 5 ng of total RNA. The line on each bar graph indicates standard deviation among three technical samples. Abbreviations: w5d=wild-type mycelia grown in GYEB for 5 days and w4h=wild-type mycelia grown in GYEB for 4 hours.

We transformed PCR products corresponding to the 3.8-kb-long LME overexpression construct generated with M13 forward and M13 reverse primers. We acquired 34 transformants from a single transformation experiment (Table 2). We examined RNA expression levels for the target gene with eight transformants in an A. brassicicola wild-type background in the order of emergence from the transformation plates. According to the real-time quantitative reverse transcription PCR(RT-QRTPCR), three of six mutants expressed 10- to 20-fold more transcripts, whereas others produced approximately threefold or similar amounts of transcripts compared with the wild type (FIG. 6A). The same overexpression construct was used to transform A. alternata to produce 15 transformants. Three of eight examined transformants produced 25- to 30-fold more transcripts than the A. alternata wild type (FIG. 6B).

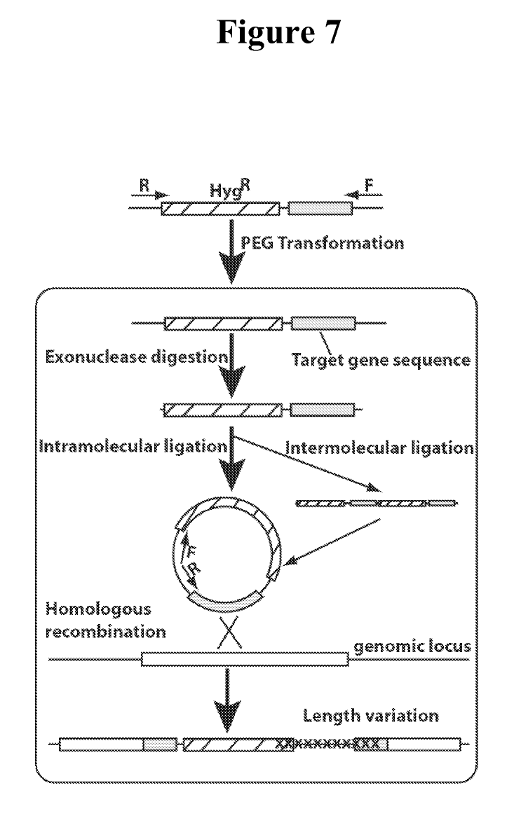

Summary Of Data Presented In This Example: The combination of partial target gene sequences disrupted by vector sequences as well as the orientation of vector sequences in transformants indicates that targeted gene disruption in our system was accomplished by a single homologous recombination preceded by circularization of the linear construct, and not by nonhomologous DNA end joining. We hypothesize that multiple steps of enzymatic activity occur during the incorporation of the disruption construct into the genome. From our data, we inferred that endogenous exonucleases are involved in the gradual digestion of linear constructs from both ends, followed by intramolecular ligation to make circular DNA (FIG. 7). More specifically, FIG. 7 shows a model of the molecular mechanism of targeted gene disruption via homologous recombination with a linear minimal element (LME) disruption construct according to an embodiment of the invention. All enzyme reactions were inferred to occur in the fungal cells (protoplasts) after DNA uptake.

In three (ace1, ace7, and ace8) of nine mutants for N-acetylglucosaminidase gene mutants, intermolecular ligation preceded intramolecular ligation, although we do not rule out a possibility of multiple homologous recombination events. However, none of these complications affect the production of targeted knock-out (KO) mutants to a great degree and should not be considered problematic. With the LME constructs consisting of minimal components, we were able to produce transformants in every trial with approximately 20 genes. We were able to generate approximately 15 transformants per construct with 10 ug of purified PCR products in approximately 4×10⁶ protoplast with an optimized protocol in three A. brassicicola isolates tested. Targeted gene disruption efficiency was improved dramatically with the LME constructs compared with standard plasmid disruption constructs. These results may be attributed to the small size of the linear constructs or intrinsic differences between plasmids and linear DNA. The typical minimal linear construct (2 kb) is approximately threefold smaller than the plasmid (5.5 kb) used in this study. Alternatively, the linear DNA might enhance both transformation and targeted gene disruption efficiencies previously shown in A. alternata (Shiotani and Tsuge 1995) due to uncharacterized reasons. However, substantial differences in transformation efficiency were not observed between LME and plasmids disruption constructs. The major difference observed between the two methods was in reliable targeting efficiency.

In most cases, gene disruption efficiency was 100% in three A. brassicicola isolates with the LME disruption construct. Importantly, the majority of mutants contain a single copy of transforming DNA. Our results show greatly increased efficiency compared with examples of approximately 40% gene disruption efficiency in other systems with either Agrobacterium-mediated transformation or transposon-mediated library construction. Moreover, our results are comparable with the targeted gene disruption efficiency of N. crassa in nonhomologous DNA end-joining machinery mutant background, and of Alternaria alternata with linearized disruption constructs. Although both examples showed 100% gene targeting efficiency, the LME disruption method has merits over them in its simplicity to create the constructs and isolate pure mutants, and in the tendency of a single-copy insertion of the disruption constructs into the genome. In addition, similar constructs are applicable to generate GPF-tagged disruption mutants (FIG. 5) and targeted-gene overexpressing mutants (FIG. 6). The former could be useful, for example, when examining interactions of mutants with host plants in downstream pathogenicity assays, whereas the latter could be useful, for example, for complementation of preexisting mutants or identification of subtle virulence factors through a gain of function approach.

This method is applicable for, among other things, the integration of EST or genome sequence information with the functional analysis of each gene by a reverse genetics approach. Combined with PCR-based construct generation, it is now possible to use LME constructs for an initial high-throughput screen to study the effect of individual genes on biological functions. It is conceivable to make a synthetic oligo library for every single gene identified in silico, followed by PCR amplification of the target gene with a selectable marker gene.

In this study, we have described a relatively straightforward, extremely efficient method for disrupting genes in Alternaria, and in particular A. brassicicola. This approach is quite suitable for development of a high-throughput functional genomics platform for analyzing gene function in this economically important organism. Other than the role of host-specific fungal toxins in pathogenesis, our basic understanding of the subtleties underlying interactions of plants with necrotrophic fungi is still in its infancy. For example, it is becoming apparent that only specific gene family members encoding secreted plant cell wall-degrading enzymes are required for full virulence of necrotrophs such as A. alternata and Botrytis cinerea on specific plants and plant parts. The high-throughput approach described here will allow for the systematic functional analysis of large sets of candidate genes such as those predicted to encode cell wall-degrading enzymes and other genes of interest identified through bioinformatic analysis of the A. brassicicola genome sequence. In summary, the method reported here undoubtedly will aid in the identification of fungal proteins and secondary metabolites representing major players at the host-pathogen interface in both compatible interactions with cultivated Brassicas and various interactions with Arabidopsis ecotypes and mutants.

### Example 2

### Knock-Out of a Non-Ribosomal Peptide Synthetase Gene

A complex and fascinating aspect of fungal biology is the production of secondary metabolites. Fungal secondary metabolites are considered part of the chemical arsenal required for niche specialization and have garnered intense interest by virtue of their biotechnological and pharmaceutical applications. Some secondary metabolites are well known virulence factors in fungal-plant interactions, causing crop damage, yield losses, and food supply contamination. Nonribosomal peptide synthetases (NPS) are large, multifunctional enzymes typically comprised of numerous semiautonomous catalytic domains in a linear series. The domains are arranged in a predictable distance from each other and in a characteristic sequence that reflects the order of their activity in the assembly and tailoring of the peptide or peptide-containing product. A minimal NPS module is composed of domains that catalyze the single reaction steps like activation, covalent binding, optional modification of the incorporated monomer substrate, and condensation with the amino acyl or peptidyl group on the neighboring module. Generally, the number and order of modules present in a NPS determine the length and structure of the resulting NRP. The activation domain recognizes a substrate amino (or hydroxy) acid, usually specifically, and activates it as its acyl adenylate by reaction with ATP. This active ester is then covalently linked as its thioester to the enzyme-bound 4′-phosphopantetheine located within the module. The reaction continues by the direct transfer to another acylamino acid intermediate on the adjacent downstream module mediated by the condensation domain to form a peptide bond. In some cases, modifications (epimerization, N- or C-methylation or cyclization) are catalyzed by additional domains or by modified domains within a module. In some NPSs, a thioesterase domain is found at the C-terminal end of the protein and is thought to release the NRP from the NPS by cyclization or hydrolysis.

Fungal NRPs have been found to have a wide range of biological activity from being involved in plant pathogenesis to the production of beneficial antibiotics. Although the production of nonribosomal peptide-associated metabolites by the thiotemplate mechanism is well supported in the literature, the biological significance and benefit of these molecules to the producing organisms remains elusive. However, evidence has been presented demonstrating that NRPs may function as signal molecules for coordination of growth and differentiation. NRPs may also participate in the breakdown of cellular metabolic products. Some NRPs, such as penicillin, clearly have antimicrobial activity and kill competing microorganisms. Other NRPs act as siderophores and assist in iron uptake. Finally, NRPs may serve as host virulence factors often possessing phytotoxic activities.

The genomic sequences of over fifty fungi have been determined, including the fungi Neurospora crassa, Aspergillus nidulans, Stagonospora nodorum, and Magnaporthe grisea, to name a few. As more genomes become available and are analyzed for their NPS genes, it will be interesting to see how the types and numbers of NPS genes (as well as secondary metabolite-associated genes in general) correlate with ecological niche and the interaction with their host, either animal or plant. Here we report on use of a nucleic acid construct of the invention to identify an NPS gene, AbNPS2, from A. brassicicola, which is involved in conidiation and more specifically conidial cell wall stabilization. This is the first report that a fungal NPS is associated with conidial cell wall construction. The role of this gene in fungal development and plant pathogenesis is also discussed.

Materials And Methods: Unless otherwise noted herein, the materials and methods used in this Example are those disclosed above for Example 1.

Fungal Strains, Media, And Fungal Culture: A. brassicicola isolate ATCC96866, the isolate used for whole genome shotgun sequencing, was used in this study. A. brassicicola was cultured on 3.9% (w/v) potato dextrose agar (PDA) (Difco, Kansas City, Mo., USA) and 1% (w/v) glucose 0.5% (w/v) yeast extract (GYEB) broth. Fungi were grown at 25-C in the dark for both solid and liquid culture.

DNA Isolation And Southern Hybridization: A. brassicicola was cultured for 2-3 days in 50 ml GYEB media. Approximately 0.2 g mycelia was harvested and filtered with Miracloth (Calbiochem, Darmstadt, Germany), semi dried with paper towels, and ground into fine powder with a mortar and pestle in the presence of liquid nitrogen. Total genomic DNA from was extracted using Plant DNeasy kit (Qiagen, Palo Alto, Calif.). A total 2-3 ug of genomic DNA was digested with an endonuclease BsrGI (New England BioLab, Beverly, Mass.). The digested DNA was size-fractionated on a 0.7% agarose gel, followed by overnight transfer to a Hybond N⁺ nylon membrane (Amersham Pharmacia Biotech, Buckinghamshire, UK). The transferred DNA was U.V. cross-linked at 120 mJ (Spectronics corporation, Westbury, N.Y.) to the membrane and subsequently hybridized with 0.5 kb long probes that were amplified from A. brassicicola genomic DNA using PCR DIG Probe Synthesis Kit (Roche Diagnostics, Mannheim, Germany). The entire procedure from the hybridization to the signal detection was carried out with Block and Wash Buffer Set and CDP-Star in the DIG Detection Kit (Roche Diagnostics, Mannheim, Germany) according to the manufacturer's protocols with following specifics. Hybridization was performed at 50-C. After the hybridization, the membrane was briefly rinsed three times at room temperature in wash solution 1 (1×SSC, 0.1% SDS) and stringently washed at 68-C in wash solution 2 (0.1×SSC, 0.1% SDS) for 30 minutes.

RNA Isolation And Northern Hybridization: Total RNA for expression analysis was prepared from fungal mycelia grown under the following condition: shake liquid glucose-yeast extract broth (GYEB) medium, 25-C, 72 hours for germination and vegetative growth. Conidiating aerial structures containing aerial mycelia, conidiophores and conidia were collected at 8 hours and 16 hours post-incubation as follows: about 20 mycelial balls collected from the above 72 hour liquid culture were spread onto sterilized filter paper and incubated for conidiation. Conidia were harvested from strains grown on glucose-yeast extract agar (GYEA) plates for 5 days at 25° C. and were washed with sterile water 3 times and collected by centrifugation for RNA extraction. For expression analysis for different fungal developmental stages 100 ul of conidial suspension (5×10⁵ conidia/ml) was spread onto GYEA plates and incubated for germination and conidiation. After 4, 8, 12, 24 and 36 hours after incubation, conidia that did not germinate or show relatively late growth rate were removed under the microscope and the conidia and colonies growing well on GYEA were collected and ground in the presence of liquid nitrogen.

Total RNA was extracted using Plant RNeasy Kit (Qiagen, Palo Alto, Calif.), followed by DNase digestion for 15 minutes at 37° C. using DNase Mini Kit (Promega, Madison, Wis.). Total RNA (20 ug) was fractionated on a 1.5% formaldehyde agarose gel and the gel was stained with ethidium bromide to assess quality and quantity of the RNA. The fractionated total RNA was transferred overnight from the agarose gel onto a Hybond N⁺ nylon membrane (Amersham Pharmacia Biotech, Buckinghamshire, UK). Hybridization and detection procedures after the RNA transfer were carried out as described in the Southern hybridization section except that hybridization and washing was carried out at 42° C.

AbNPS2 Promoter And GFP Fusion-Protein Construct: Two primers were designed at 1 bp position with an exogenous ApaI site (5′-tagggcccATGGTGAGCAAGGGCGAGGA-3′; SEQ ID NO:9) and at 2370 bp position with an exogenous Hind III site (5′-acaagcttTGGTTCCCGGTCGGCATCTA-3′; SEQ ID NO:10) in relation to the GFP start codon. These primers were used to amplify GFP coding region and Hyg B cassette from template plasmids pCG16G6-Nac. The PCR products were cloned at the corresponding multiple cloning sites in pBluscript (SK+) to create pCB16G6-PF. Another set of primers were designed at −1 bp position (upstream of start codon) with an exogenous ApaI site (5′-gagggcccCGTGGGCCGTGTGTGGTTTC-3′; SEQ ID NO:11) and at approximately the −1000 bp position with an exogenous KpnI (5′-gtggtaccCAGCCTCGCAGACACTCGAC-3′; SEQ ID NO:12) in relation to the AbNPS2 start codon. These primers were used to amplify the AbNPS2 promoter region and the subsequent PCR products were cloned in the corresponding multiple cloning sites of pCB16G6-PF to make pCB16g6-N2. The pCB16G6-N2 sequentially contains 1 kb AbNPS2 promoter, a GFP open reading frame, and Hyg B cassette between M13 forward and the M13 reverse priming sites. All cloning and plasmid production were carried out as described in the following section. A fragment containing 1 kb AbNPS2-promoter region, GFP cassettes and Hyg B cassettes were amplified from pCB16G6-N2 with M13 forward and M13 reverse primers. The PCR products were transformed in the wild-type A. brassicicola to make AbNPS2 promoter-GFP fusion mutants.

Generation Of Targeted Gene Disruption Construct And Fungal Transformation: Based on AbNPS2 sequence identified in the partially assembled A. brassicicola genome sequence, two primers were designed at the 911 bp position with an exogenous enzyme site HindIII (5′-cttgaagcttTCCTTCCTGCTGTCGATGTT-3′; SEQ ID NO:13) and at the 1441 bp position with an exogenous XbaI site (5′-ccattctagaATGCGTCTGGGAATTGGCAC-3′; SEQ ID NO:14) in relation to the putative start codon. These primers were used to amplify a 550 bp partial target gene from the genomic DNA. PCR products were digested with HindIII and XbaI, and ligated at the corresponding multiple cloning sites in pCB1636. The ligation was transformed in E. coli strain DH5alpha (Invitrogen, Carlsbad, Calif.). The plasmid was isolated via miniprep (Qiagen, Palo Alto, Calif.) and sequence verified for the presence of insert and Hyg B cassette and used as templates for PCR amplification using M13 forward and M13 reverse primers. The PCR product was purified with PCR Cleanup kit (Qiagen, Palo Alto, Calif.) and further concentrated to 1 ug/ul under vacuum before fungal transformation. Fungal transformation was carried out with linear PCR products based on the transformation protocol described above.

Surface Hydrophobicity Assay: The strains to be assayed were plated onto PDA and incubated at 25° C. until sporulation. Sterile distilled water (30 ul) was placed on the surface of cultures and the plates were incubated for 30 minutes at room temperature (RT).

Electron Microscopy: Conidia of wild-type strain ATCC96866 and Abnps2 mutant AbN2-1 were released in sterile water from 7-day-old and 21-day-old PDA plates and collected by centrifugation at 5000×g for 10 minutes. The conidial pellet was coated with 0.8% agarose and fixed in modified Karnovsky's fixative containing 2% paraformaldehyde and 2% (v/v) glutaraldehyde in 0.05 M sodium cacodylate buffer (pH 7.2) overnight at 4° C. After washing three times with 0.05 M sodium cacodylate buffer (pH 7.2) for 10 minutes each, samples were post-fixed with 1% (w/v) osmium tetraoxide in the same buffer for 2 hours at 4° C. The post-fixative was removed by washing briefly twice with distilled water at room temperature and the samples were en bloc stained with 0.5% uranyl acetate overnight at 4° C. Then the samples were dehydrated in a graded ethanol series and embedded in Eppon resin. Ultrathin sections cut from the Eppon-embedded material with ultramicrotome (MT-X, RMC, USA) were collected on carbon-coated grids, stained with 2% uranyl acetate for 3 minutes, and with Reynold's lead solution (Reynolds, 1963) for 3 minutes. Examination was conducted with a JEM-1010 (JEOL, Tokyo, Japan) electron microscope operating at 60 kV.

Confocal Microscopy: AbNPS2 promoter-GFP fusion transformants were grown on GYEA plate and in GYEB broth, and prepared exactly the same as the RNA blotting samples for the confocal microscopy. Inverted laser scanning microscope (LSM-510, Carl Zeiss, Göttingen, Germany) and an argon ion laser for excitation at 488 nm wavelength and GFP filters for emission at 515-530 nm were used for this experiment. The imaging parameters used produced no detectable background signal from any source other than from GFP. Confocal images were captured using LSM-510 software (version 3.5; Carl Zeiss) and were recorded simultaneously by phase contrast microscopy and fluorescent confocal microscopy. Phase contrast images were captured with a photomultiplier for transmitted light using the same laser illumination for fluorescence.

Conidiation Assays: The conidiation rates were compared between the wild-type, abnps2 mutants, and ectopic insertion mutants. We examined the rates during the development in vitro on PDA plates and in vivo on plants. For the in vitro assay, fungi were cultured on PDA plates with a photoperiod of 16 hours using fluorescent lights for 7 days and conidia were harvested in sterile water. For the in vivo assay 7 day-grown 1000 conidia in 10 ul water were inoculated on detached green cabbage leaves, followed by 7 days of incubation at room temperature with 100% relative humidity under long-day conditions (16-hours light/8-hours dark cycle). Conidia from a size of 1 cm² infected leaf fragment were released in 5 ml water. Conidia produced on lesions were harvested by vortexing the test tube vigorously. Leaves were removed, and the conidia-containing suspensions were centrifuged at 5000×g for 15 minutes. The conidia were resuspended in 200 ul of water, serially diluted, and counted using a hemacytometer.

Germination Rate Comparisons: Conidial germination was measured on cover glasses (Fisher Scientific, Hampton, N.H., USA) and plant leaf surfaces. Conidia were harvested from 7-day-old, 14-day-old and 21-day-old cultures on PDA in sterile distilled water and adjusted to 1×10⁴ conidia per ml. Drops (30 ul) were placed on cover glasses and detached leaf segments, then placed in a moistened box and incubated at RT for 36 hours on cover glasses and 8 hours on detached leaf segments. In vivo conidial germination was measured as follows: the leaf fragments with conidial drops were transferred into a test tube containing 2 ml of water, the test tube was vortexed vigorously to release conidia, and conidia were harvested from the test tube by pipetting. The percentage of germinated conidia was determined by microscopic examination of at least 100 conidia per replicate in at least three independent experiments, with three replicates per treatment. Statistical analyses were performed to test the differences in germination rates among the four strains by least significant difference pairwise comparisons (p≦0.05).

Virulence Tests: To test virulence, conidia of fungi were harvested from PDA agar plates incubated for either 7 days, 14 days or 21 days at 25° C., and suspended in sterile water at a concentration of 5×10⁴ conidia per ml. Conidial suspensions (10 ul) were applied as drops on the surface of middle-aged leaves (fifth through sixth leaf stages). Inoculated plants were placed in a plastic box at RT and incubated at 100% humidity for 24 hours in the dark, and then followed with a photoperiod of 16 hours using fluorescent lights for 4 days. Lesion diameters were then measured. Statistical analyses were performed to test the differences in lesion diameters among the four strains by least significant difference pairwise comparisons (p≦0.05).

Results:

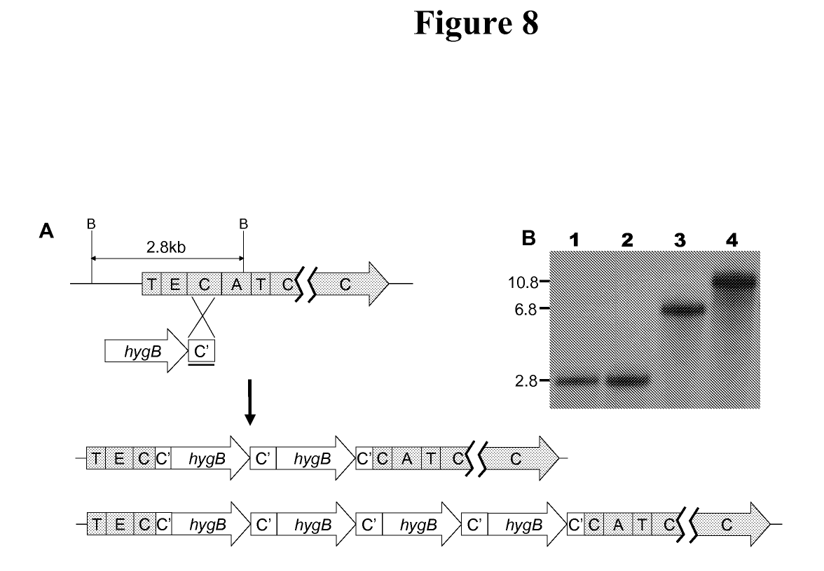

Targeted disruption of AbNPS2: To investigate the function of AbNPS2, we generated six abnps2 null mutants by disrupting the target gene using linear minimal element (LME) constructs (see FIG. 8A). Two of the six mutants were randomly selected for phenotypic characterization in this study. PCR was initially used to identify transformants disrupted at the AbNPS2 locus as well as ectopic mutants (data not shown). Based on these results, two targeted gene disruption mutants and an ectopic insertion mutant were verified by Southern hybridization (FIG. 8). Consistent with the PCR results, the wild-type hybridization pattern consisted of a single band (2.8 kb) shifted to 6.8 kb and 10.8 kb in AbN2-1 and AbN2-2 transformants, indicating targeted gene disruption by two and four copies of the LME constructs in each mutant, respectively (FIG. 8B). We confirmed no additional integration of the constructs in any other location in the genome by Southern hybridization with Hyg B probes (data not shown). In addition, we qualitatively assessed stability of abnps2 disruption mutants by 5 sequential hyphal tip transfers to fresh PDA plates lacking Hyg B in approximately one week intervals. The repeated subculturing on PDA media did not change Hyg B resistance of the abnps2 mutants during the repeated hyphal tip transfers after five generations.

More specifically, FIG. 8 shows data indicating that targeted disruption of the AbNPS2 gene was accomplished. Panel A: shown are wild-type AbNPS2 gene locus, a disruption cassette, and two loci disrupted by the cassette. The disruption cassette is a linear minimal element (LME) construct comprised of a hygromycin phosphotransferase (Hyg B) cassette and a partial target gene sequence. Two mutated genomic loci, AbN2-1 and AbN2-4, are depicted to show two and four tandem insertions of LME constructs, respectively. Panel B shows Southern blot analyses sequentially showing wild-type, ectopic insertion, and two targeted gene disruption mutants. The letter B on the genomic locus indicates enzymatic sites for BsrGI that were used for genomic DNA digestion. The region used for labeling the hybridization probe is marked with a bar under the LME disruption construct. Abbreviations: A, adenylation; C, condensation; E, epimerization; T, thiolation.

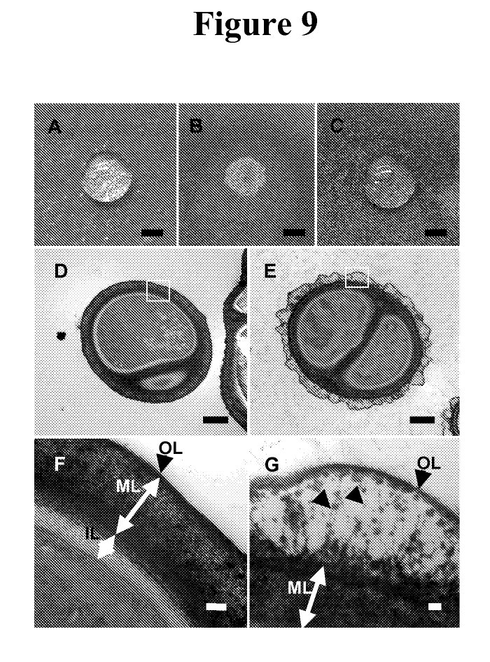

Abnps2 Mutants Show Decreased Hydrophobicity Phenotype And An Aberrant Conidial Cell Wall: Typical wild-type conidia of A. brassicicola are very hydrophobic. When water drops were placed on the lawn of aerial hyphae bearing conidia, they remained beaded and easily rolled around on the surface for up to 10 hours. In contrast, water drops on the abnps2 mutants at a similar developmental phase were immediately absorbed through the lawn of aerial hyphae and conidia (see FIG. 9, Panesl A-C). In addition to the rapid absorption of the water, the mutants showed a slightly vagarious conidial surface discernable from the wild-type under a phase contrast light microscope (data not shown). Therefore, we decided to investigate the aberrant morphology of the mutant conidia using transmission electron microscopy (TEM). For the wild-type conidia the outermost layer appeared smooth and the cell wall structure appeared compact (FIGS. 9 D and F). In contrast, the outermost layer of abnps2 mutant conidia appeared fluffy and the outermost layer of the cell wall structure was separated from the middle electron dense layer (FIGS. 9E and G). There were filamentous structures, loosely connecting the detached outermost layer with the middle layer (FIG. 9G). Based on this data, it appears that the AbNPS2-derived metabolite product is involved in cell wall formation and/or cell wall architecture. Specifically, it appears as though the AbNPS2-derived metabolite product is a component or facilitates the linkage of the outermost layer and the middle layer of the fungal spore cell wall.

With respect to FIG. 9, the figure shows the decreased hydrophobicity phenotype of the abnps2 mutant, along with electron micrographs of the abnps2 mutant. More specifically, the top panel pictures depict the fate of 30 ul water drops deposited on PDA plates covered with conidia. Panel A shows the wild-type conidia, which show a hydrophobic surface. Panel B shows an AbN2-1 mutant conidia showing a water wettable surface. Panel C shows condia from an ectopic insertion mutant, showing a similar hydrophobic phenotype as the wild-type. The four pictures of Panels D-G are transmission electron micrographs depicting the aberrant ultrastructure of abnps2 mutants (E and G), compared to the wild-type (D and F). Panels F and G show magnification of the rectangles in Panels D and E, respectively. Arrowheads point to microfibrils in the middle cell wall layer. Bars indicate 20 mm (A, B, C), 2 um (D, E), and 200 nm (F, G). Abbreviations: OL=outermost cell wall layer, ML=middle cell wall layer, IL=inner cell wall layer.

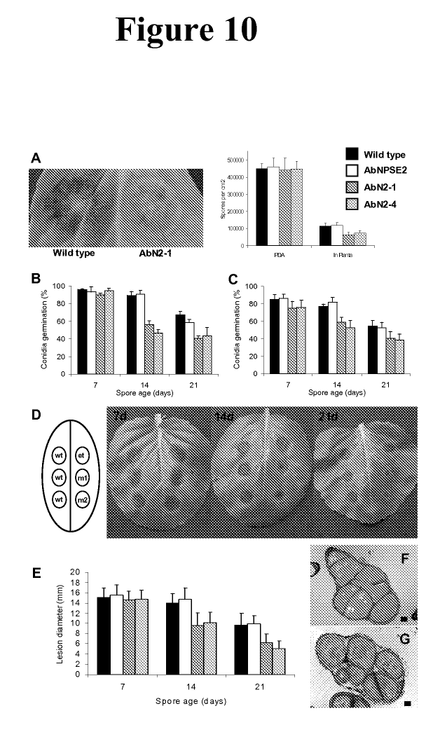

AbNPS2 Is Important For Conidial Viability And Virulence: Virulence and conidia production were compared among two abnps2 mutants, an ectopic insertion mutant and the wild-type in vivo on the host plant (green cabbage) leaves. When approximately 1000 (7 days old) conidia of each strain collected from PDA plates were inoculated onto detached plant leaves, the difference was negligible in the size of necrotic lesions formed. However, the disruption mutants produced approximately 40% less conidia than the wild-type and the ectopic insertion mutant (FIG. 10A). We further examined the mycelial growth rate and conidia production in vitro on PDA plates. However, there were no significant difference in conidial production and mycelial growth rates among all three strains (FIG. 10A). This result suggested that abnps2 mutant is more sensitive to biological stresses imposed during plant infection than the wild-type because of its cell wall abnormalities.

To understand the biological significance of the mutant phenotype with the abnormal cell wall structures, we examined the germination rate of the mutant conidia that were collected from PDA plates after 7, 14, and 21 days of culture incubation (DCI). On cover glasses, 7- and 14-day-old wild-type conidia germinated over 90% and the 21-day-old wild-type conidia had a 60% germination rate. There was no significant difference in the germination rates for the ectopic insertion mutant and the wild-type conidia. Germination rates of the abnps2 mutants were lower than both the wild-type and the ectopic mutant in all three aged groups (FIG. 10). It is especially noteworthy that the germination rate precipitously decreased to 50-58% for the 14-day-old abnps2 mutants and further decreased to 40-45% level for 21-day-old mutants. The trend of the observed decreased germination rates in vitro was similar in vivo although the average germination rate of all tests was somewhat lower than the in vitro assay in general.

Germination rates became progressively lower as the conidia aged on the PDA plates for both wild-type and mutants. The difference between the wild-type and the mutant was in the efficiency of the germination rates. To investigate the effects of decreased germination rate in regards to pathogenicity, virulence assays were carried out with conidia collected from PDA plates after 7, 14 and 21 DCI. After the in planta inoculation of 500 conidia in 10 ul water, the disease severity was estimated by measuring the lesion size. Plants inoculated with conidia from 7-day-old culture of the wild-type strain and abnps2 mutants developed almost the same size of typical black spot lesions on the leaves, but inoculations performed with 14-day-old abnps2 mutants resulted in formation of a statistically significant smaller lesion size compared to the wild-type of same aged group. A significant reduction in lesion size on the individual leaves was even more evident in 21-day-old abnps2 mutants 5 days after inoculation compared to the wild-type (FIGS. 10D and E). In consideration of the possibility that the reduced pathogenicity of the aged mutants could result from other defectiveness of the abnps2 mutant during infection, we closely investigated the infection process using electron microscopy. Following initial germination there were no observed differences between the wild-type and abnps2 mutants in appressoria formation and penetration into plant epidermal cells (data not shown).

Conidia collected from PDA plates after 21 DCI were examined using transmission electron microscopy to understand the possible reason for the reduction in conidial germination rate over time in abnps2 mutants compared to the wild-type. The wild-type conidia were filled with typical cellular organelles, glycogen, and lipid droplets. The lipid granules were few in number and small in size, and an intact plasma membrane was visible (FIG. 10F). In abnps2 mutant conidia, however, the lipid granules were largely expanded and occupied almost the entire cell (FIG. 10G). The intercellular walls between each conidial cell component appeared relatively weakened compared to the wild-type resulting in a rounder shape of each cell similar to protoplasts that lack a cell wall.

A detailed description of the panels in FIG. 10 is as follows: In general, FIG. 10 shows that there is reduced virulence associated with reduced germination rate in abnps2 mutants. Panel A shows a conidiation test on PDA and in vivo. The picture on the left shows the conidiation in vivo of wild-type and AbN2-1 mutant strain 7 days post inoculation. Panels B and C show a germination test in vitro (Pane B) and in vivo on green cabbage (Panel C) of wild-type, ectopic, and abnps2 mutant strains. Panels D and E show a pathogenicity assay on green cabbage leaves using conidia from 7-day-(7 d), 14-day-(14 d), and 21-day-old (21 d) culture of wild-type, ectopic, and abnps2 mutant strains. The diagram (left side of Panel D) indicates inoculation sites of each strain. Panels F and G show Electron micrographs of 21-day-old wild-type (F) and abnps2 mutant (G) conidia. Note that the wild-type conidia were normally filled with cellular organelles, glycogen, and lipid droplets, and intact intercellular walls are visible (Panel F). The lipid granules were expanded and intercellular walls aberrant in abnps2 mutants compared to the wild-type (Panel G). Bars indicate 2 um. Columns and error bars on graphs represent averages and SD, respectively, of four independent experiments (A, B, C, and E). Statistical analyses were performed to test the differences in germination rates and lesion diameters among the four strains by least significant difference pairwise comparisons (p≦0.05).

Discussion Of Example 2 Data:

AbNPS2 Expression Is Related To Conidiation, A Reproductive Developmental Phase: Filamentous fungi undergo distinct life-cycle phases of growth (accumulation of undifferentiated hyphae) and reproduction (elaboration of fruiting structures). Switching between these two phases is highly regulated and initiation is governed by perception of a combination of physiological and environmental conditions and cues. AbNPS2 promoter-GFP fusion analysis and RNA blot analysis clearly showed that the pattern of AbNPS2 expression is related to the conidiation process. The morphological changes associated with the reproductive phase were coupled with AbNPS2 gene expression and the putative production of a yet to be identified secondary metabolite produced via AbNPS2 and the products of clustered modifying genes. It has been reported that fungal secondary metabolism and sporulation (conidiation) are associated both temporally and functionally. Illustrating the latter, secondary metabolites in Aspergillus and Fusarium (including the mycotoxin zearalenone) are associated with the onset of sporulation. Some secondary metabolites act as pigments protecting spores. Sterigmatocystin is a fungal secondary metabolites that appears to be important for sporulation in A. nidulans. For example, functional analysis of NPS6 in C. heterostrophus revealed that the product of this NPS might protect the fungus from oxidative stress and is critical for full virulence

AbNPS2 Might Synthesize A Conidial Cell Wall Component: AbNPS2 is related to surface hydrophobicity of conidia, as shown in FIG. 9. Disruption of AbNPS2 resulted in a water-soaked, easily wettable phenotype in vitro. Similar phenotypes were reported in several hydrophobin deletion mutants in fungi, which suggested that the synthesized metabolic product of AbNPS2 might be related to a process of hydrophobic coating of fungal aerial structures. In addition to the rapid absorption of the water, however, the mutants showed a slightly vagarious conidial surface discernable from the wild-type under a phase contrast light microscope, which has never been reported for any hydrophobin-disrupted mutant. Therefore, we turned our interest to the conidial cell wall itself instead of the surface hydrophobic proteins of conidia.

The cell walls of most filamentous fungi have a fibrillar structure consisting of chitin, beta-glucans, and a variety of heteropolysaccharides. Targeted mutation of AbNPS2 resulted in aberrant conidial cell walls in A. brassicicola. It is very rare to obtain discernable morphological phenotypes associated with expression of the NPS in filamentous fungi. Furthermore most NPS products described thus far have been secreted molecules, such as antibiotics, siderophores, and toxins, but not structural components. The pigment melanin was the only cell wall component encoded by secondary metabolite-producing genes in fungi reported thus far. Transmission electron microscopy showed that the outermost layer of conidial cell wall of abnps2 mutant was separated from the original cell wall body. Our microscopic studies revealed that three layers were recognized in sections of conidia of the A. brassicicola wild-type strain: the innermost one electron transparent, the middle layer appearing electron dense, and the outermost layer which was very thin (15 to 20 nm thick) and also quite electron dense. This cell wall organization usually results from the intimate association of the different constituents through hydrogen bonding, hydrophobic and electrostatic interactions and even by the establishment of covalent bonds, all of them occurring in a rather precise and specific manner. The electron micrographs demonstrate that the middle layer contains substantially larger amounts of amorphous compounds and intertwining microfibrils, which stain more or less strongly, whereas the outermost layer had a granular or amorphous structure of high electron density. In general, reports suggest that fibrillar polysaccharides are accumulated mostly in the inner layers of the cell walls, whereas glycoproteins are more abundant in the external layers. Researchers have concluded that the external coat was made of amorphous beta-glucans placed over a reticulum of glycoproteins. More internally, it was suggested, a protein layer followed where chitin microfibrils were embedded. Based on our data and these reports, it is possible that the abnps2 deletion mutants were lacking a compound connecting or attaching the glycoprotein and/or amorphous compounds in the outermost protein layer to the microfibrillar polysaccharides in the outer part of the middle layer. The presence of residues of the fibrillar net between the middle layer and the outermost layer in FIG. 9G supports the notion that the role of the secondary metabolite produced by AbNPS2 is to stabilize and make the conidial cell wall more rigid, resulting in conidia with an improved ability to survive in adverse environmental conditions.

Implications Of AbNPS2: The mechanical and osmotic stability of most plant and microorganism cells is achieved by their cell walls, constructed of different polysaccharide moieties to which various proteins are attached. The fungal cell wall is critical for cell viability and pathogenicity. Beyond serving as a protective shell and providing cell morphology, the fungal cell wall is a critical site for exchange and filtration of ions and proteins, as well as metabolism and catabolism of complex nutrients. Disruptions of this protein/carbohydrate cell wall matrix have resulted in high sensitivity to osmotic lysis of fungal cells. Disruption of AbNPS2 resulting in cell wall abnormalities did not affect the osmotic resistance but did affect UV tolerance. Treatment with UV resulted in a 25% decrease in germination rate of the abnps2 mutants compared to the wild-type (data not shown). In addition to environmental stresses, biological stress also affected the abnps2 mutants. The reduced conidial reproduction in vivo suggests that abnps2 mutant is more sensitive to biological stress than the wild-type. In FIG. 9A, we observed the lesion size of the wild-type and abnps2 mutants were highly similar, demonstrating the vegetative invasive growth of the wild-type and abnps2 mutants in vivo was the same but only when inoculations were performed with young conidia. However, the factors in green cabbage causing reduced conidial reproduction rate of abnps2 mutants remains to be revealed. The abnps2 mutants were tested for increased sensitivity in vitro to the Arabidopsis antimicrobial phytoalexin camalexin, but no differences were observed between mutants and wild-type (data not shown).

As conidia aged in vitro, the frequency of conidial germination decreased in abnps2 mutants, but the wild-type maintained a germination rate at a relatively high level in each test group compared to abnps2 mutants. These germination assay results were consistent in in vitro and in vivo assays, suggesting that pathogenicity of older conidia of abnps2 mutants might be less virulent mainly due to a decrease in germination rate. In some previous experiments, it was reported that decreased germination rate resulted in reduced pathogenicity. In fact, virulence tests with abnps2 mutants revealed a significant reduction in lesion size compared to the wild-type when aged spores were used in experiments. This result is supported by ultrastructural analysis demonstrating that 21-day-old abnps2 mutant conidia were abnormal. Autolysis-like phenomena had occurred in the conidia of abnps2 mutants, in which formation of numbers of lipid bodies and degradation of intercellular wall occurred. Although the precise biochemical mechanism underlying these phenomena is not known, similar phenomena have been reported in other fungi. These include mature basidiospores with fully grown hilar appendix and old aged fungal hyphae grown on the media lacking nitrogen. In addition, five different genera of filamentous fungi were incubated for 60 days, which resulted in 23.5-87.3% degree of cell autolysis. The secretion of the lytic enzymes was consistent with the degree of autolysis in each fungus. Our data suggests that the presence of many lipid bodies and the degradation of conidial cell wall in abnps2 mutants resulted from premature aging of conidia accompanied with autolysis-like phenomena. The relatively weak cell wall of abnps2 mutants might not be sufficient to prevent premature aging of conidia. Fungal cells have significant internal turgor pressure and thus undergo lysis when their cell walls are even slightly perturbed. However, whether the outermost cell wall layer affects conidial viability remains unanswered. In addition, our evidence showing that high numbers of abnormally large lipid bodies filling the cytoplasm of mutant conidia are related to cell autolysis has not been reported yet in any microorganisms. Although further experiments should be performed to answer these questions, we speculate that the relatively rapid decrease in germination rate and pathogenicity of the abnps2 mutant is due to loss of a cell wall component or cell wall integrity in general. In conclusion, AbNPS2, a gene encoding an NPS synthesizing a secondary metabolite, plays significant roles in conidial cell wall construction and conidial development. This might be one physiological mechanism associated with the Dothideomycetes, a taxonomic group which harbors many important plant pathogenic genera, that allows for enhanced fitness and to preserve its asexual structures in space and time.

### Example 3

### Analysis of the Fus3/Kss1 MAP Kinase Homolog Amk1

Mitogen-activated protein (MAP) kinases have been shown to be required for virulence in diverse phytopathogenic fungi. To study its role in pathogenicity, we disrupted the Amk1 MAP kinase gene, a homolog of the Fus3/Kss1 MAP kinases in Saccharomyces cerevisiae, in the necrotrophic Brassica pathogen, Alternaria brassicicola. The amk1 disruption mutants showed null pathogenicity on intact host plants. However, amk1 mutants were able to colonize host plants when they were inoculated on a physically damaged host surface, or when they were inoculated along with nutrient supplements. On intact plants, mutants expressed extremely low amounts of several hydrolytic enzyme genes that were induced over 1,000-fold in the wild type during infection. These genes were also dramatically induced in the mutants on wounded plants. These results imply a correlation between virulence and the expression level of specific hydrolytic enzyme genes plus the presence of an unidentified pathway controlling these genes in addition to or in conjunction with the Amk1 pathway.

Filamentous fungal phytopathogens penetrate and cause disease symptoms on host plants by various mechanisms induced by external signals. These include the formation of differentiated structures called appressoria that physically breach the host cuticle and cell wall barriers with high turgor pressure and secretion of hydrolytic enzymes that chemically breakdown various cell wall components. The yeast and fungal extracellular signal-regulated kinase subfamily (YERK1) represented by Fus3/Kss1 in Saccharomyces cerevisiae is among a mitogen activated protein (MAP) kinase superfamily that is essential for pathogenicity of many fungal phytopathogens. MAP kinase is required for appressorium formation in fungal pathogens, such as Colletotrichum lagenarium, Magnaporthe grisea, Cochliobolus heterostrophus, and Pyrenophora teres. M. grisea and C. lagenarium produce especially large and heavily melanized appressoria that generate strong turgor pressure. In both fungi, MAP kinase (Fus3/Kss1 homolog) mutants are defective in appressorium formation and are nonpathogenic as a result of their inability to penetrate the plant epidermis as well as to colonize host plant tissues.

There is also substantial evidence supporting the importance of enzymatic digestion in lieu of or in addition to mechanical forces in host cuticle penetration and subsequent colonization. For example, several cell wall degrading enzyme (CWDE) coding genes and their control element genes have been identified as virulence factors whose null mutation significantly reduced infection abilities in diverse phytopathogens. There are also many fungal phytopathogens that do not make appressoria in the wild type but directly penetrate the leaf surface. Even in appressorium-forming fungi like C. heterostrophus and C. lagenarium, these structures are nonessential for penetration, indirectly supporting the importance of CWDE in penetration as well as colonization. Recently, it has been shown that MAP kinases induce the expression of CWDE genes in Fusarium oxysporum, C. heterostrophus, and Trichoderma atroviride and enzyme activities in F. graminearum. A. brassicicola is a causal agent of blackspot disease of cultivated Brassicas and has also been widely used as a necrotrophic pathogen of Arabidopsis. The objectives of this study are to examine the Fus3/Kss1 homolog Amk1 in A. brassicicola, regarding its functional importance in virulence and transcriptional regulation of downstream CWDE genes. There are correlations between the severity of virulence and induction of specific hydrolytic enzyme (putative CWDE) genes which are suppressed during saprophytic growth in vitro. This study results suggest dual functions of Amk1 as a positive and negative transcription regulator of putative CWDE genes during host infection and saprophytic growth in nutrient rich environments, respectively.

Materials And Methods: Unless otherwise noted, the materials and methods used in this Example are those used in Examples 1 and 2, above. Particular materials and methods are indicated as follows:

Fungal Strain, Growth Conditions, Transformation, And Targeted Gene Mutant Identification: A. brassicicola strain ATCC 96866 was used in this study (American Type Culture Collection, Manassas, Va.). Culture, transformation, nucleic acid isolation, and Southern hybridizations were performed as described above.

Generation Of Targeted Gene Knock-Out Mutants: Based on the full length cDNA sequence (AY515257) of the Amk1 gene, two primers were designed at the 334 bp position (5′-gcaagcttgagctttcggacgaccattgc-3′; SEQ ID NO:15) and the 789 bp position (5′-gtgaattccggcagcgatcgaatgtattcg-3′; SEQ ID NO:16) in relation to the start codon. Primers were designed to incorporate exogenous enzyme sites HindIII and EcoRI, respectively. PCR products amplified from cDNA were digested with HindIII and EcoRI, and ligated into the pCB1636 fungal transformation vector (Sweigard et al., 1997). The plasmid construct was transformed into E. coli strain DH5alpha (Invitrogen, Carlsbad, Calif.) to produce over 20 ug of circular plasmid disruption constructs. The gene disruption constructs were transformed into A. brassicicola either as circular plasmids or as linear minimal elements amplified from the plasmid using M13 forward and M13 reverse primers, following the selection of transformants using Hygromycin B as selectable antibiotics as described above.

In order to reintroduce wild type Amk1 into the amk1 mutant, the wild type Amk1 allele from A. brassicicola genomic DNA was amplified, which covers 2,408 bp between the 845 bp upstream in relation to the start codon and the 350 bp downstream in relation to the stop codon using a primer set, Amk5comF (5′-accttccctgtgttttgcac-3′; SEQ ID NO:17) and PNRFcAmk5R (5′-tgtgtttgtttccaagaaaagagggcattgaaggtgtagcat-3′; SEQ ID NO:18). Separately a 2076 bp long Nourseothricin-resistant cassette was amplified using a primer set, amkRcPNRF (5′-atgctacaccttcaatgccctcttttcttggaaacaaacaca-3′; SEQ ID NO:19) and pNRR (5′-tcattctagcttgcggtcct-3′; SEQ ID NO:20) from pNR plasmid as template DNA (Malonek et al., 2004). The final construct for transformation was amplified using two primers Amk5comF and pNRR from the mixture of the two PCR products as template DNA, according to the manufacturer's instruction of AccuPrime Polymerase kit (Invitrogen, Carlsbad, Calif.). The final construct was transformed into amk1 mutants, and transformants had been selected on the PDA plates containing both Hygromycin B and Nourseothricin antibiotics during transformation and two rounds of single conidia isolation.

Pathogenicity Test: Pathogenicity tests were performed on various Brassica species, B. napus, B. carinata, B. juncea, B. oleracea, and B. chinensis. For assays involving detached leaves, fresh middle-aged leaves from four to six week old host plants were removed and placed on a wet paper towel in a 50 mm Petri plate. Wild type and mutants were inoculated at the left and right sides symmetrically from the central vein. For wild type and ectopic insertion mutants, conidia were collected from one week old cultures on potato dextrose agar (PDA), counted using a hemacytometer and adjusted to various concentrations (e.g. 1×10⁵ conidia/ml) in sterile water. For the amk1 mutants, fragmented protoconidia were collected because the mutants did not produce mature conidia, and the concentration was normalized by optical density at 600 nm (OD₆₀₀). In addition, partially homogenated mycelia grown in GYEB (1% glucose and 0.5% yeast extract) media were also used for infection assays. All inocula were washed in 50 ml ddH₂O three times to minimize the effect of nutrients during the infection process. The inocula were sprayed or deposited on detached plant leaves or whole plants as a 10 ul drop in a varying concentration between 1×10⁴ and 3×10⁸ conidia or fragmented protoconidia/ml. All inoculated plants were kept in 100% humidity in semi-transparent plastic containers.

Pathogenicity Restoration Experiment: To test colonization ability of amk1 mutants, 10 ul fragmented mycelia or protoconidia in a concentration of 1×10⁵ were inoculated on needle scratched host plant leaves and incubated at room temperature (approximately 20-25° C.) for 5 days. To examine the restoration of pathogenicity by nutrient supplementation, we inoculated amk1 suspended in glucose (1%), yeast extract (1%), tryptone (1%), casamino acids (1%), bovine serum albumin (1%), and GYEB media.

Scanning Electron Microscopy: Scanning electron microscopy (SEM) was conducted on infected host leaves 24 and 48 hours after inoculation. B. oleracea and B. juncea leaf tissues infected with A. brassicicola were stabilized in Karnovsky's fixative (4% paraformaldehyde, 5% glutaraldehyde, sodium cacodylate buffer, pH 7.3) for 16 hours. After alcohol dehydration (15%, 30%, 50%, 70%, 95%, 100% for 15 minutes each), the samples were dried under CO₂ using a Critical Point Dryer (LADD, Burlington, Vt.), mounted on a SEM supporter, gold coated in 300 Å thickness using SPI-sputter coater (Structure probe Inc, West Chester, Pa.), examined, and photographed with an electron microscope (EV040, Zeiss, Gemamy) at 15 KV.

Gene Expression Survey Using RT-PCR And Semi-QRT-PCR: Detached leaves of green cabbage (B. oleracea) were 10 ul spot-infected with amk1-c and wild type, according to the infection assay protocol. Infected samples were collected at 24 hours and 34 hours after the infection with wild type. In a separate experiment, one sample was spray-infected and the sample was collected at 53 hours after the inoculation. For amk1-c infected tissues, samples were collected at 53 hours and 5 days after infection on the wounded plant tissues and 53 hours and 5 days after infection on intact plant leaves. All samples were investigated twice independently. All samples were collected from multiple leaves to reduce sample variation and immediately frozen in liquid nitrogen. For in vitro grown samples, wild type conidia and amk1-c protoconidia were cultured for 96 hours to ensure germination and healthy growth. Mycelia were collected and washed three times with sterile ddH₂O and further cultured in selected media for 4-12 hours before sample collection for RNA extraction. All RNA samples were treated with DNase I (Ambion, Austin, Tex.) before cDNA synthesis. Total RNA extraction, cDNA synthesis, and reverse transcription PCR(RT-PCR) were performed as described above, using a primer pair for each gene in Table 3. Absolute amounts of transcript were calculated using standard curve data for each gene and relative amounts of fungal RNA in the mixed tissue samples were normalized with the fungal specific Actin gene. A pair of Actin primers was designed to amplify only fungal Actin transcripts by using fungal specific sequence that differs from the Arabidopsis sequence.

TABLE 3
Primer Pairs Used For RT-PCR In Example 3

| | | SEQ |
|---|---|---|
| | | ID |
| Primer | Sequence | NO |
| Actin forward | 5′- GGCAACATTGTCATGTCTGG -3′ | 21 |
| Actin reverse | 5′- GAGCGAAGCAAGAATGGAAC -3′ | 22 |
| Alt1b forward | 5′- ACAACCTCGGCTTCAACATC -3′ | 23 |
| Alt1b reverse | 5′- AGCGACATAGGTGGTGTCGT -3′ | 24 |
| Chymotrypsin | 5′- CGGTACCACTGGAAACACT -3′ | 25 |
| forward | | |
| Chymotrypsin | 5′- TGGGTGAGACCAGTAACACG -3′ | 26 |
| reverse | | |
| Glycosyl | 5′- GTGGCATGCAATATGGACAA -3′ | 27 |
| hydrolase forward | | |
| glycosyl | 5′- GCGGTAGAAATTTGCCTTGA -3′ | 28 |
| hydrolase reverse | | |
| Lipase forward | 5′- CATTCTGGGGACGTTCCAT -3′ | 29 |
| Lipase reverse | 5′- CTGTTCGCTCCGAAGTTCAT -3′ | 30 |
| N-acetylglucosa- | 5′- ACACAGGGTAGAACCCAAC -3′ | 31 |
| minidase forward | | |
| N-acetylglucosa- | 5′- GTTTTCCCGCTGTCAAAGAA -3 | 32 |
| minidase reverse | | |
| Pectate lyase | 5′- CCACTTGACCGTCACCTACA -3′ | 33 |
| forward | | |
| Pectate lyase | 5′- GTGTTCTTGCTGTTGCCAAA -3′ | 34 |
| reverse | | |
| Cutinase forward | 5′- CGTAGAGAATGCCCTTCCAG -3′ | 35 |
| Cutinase reverse | 5′- CGACACCCTTGATTTGGTCT -3′ | 36 |

Results:

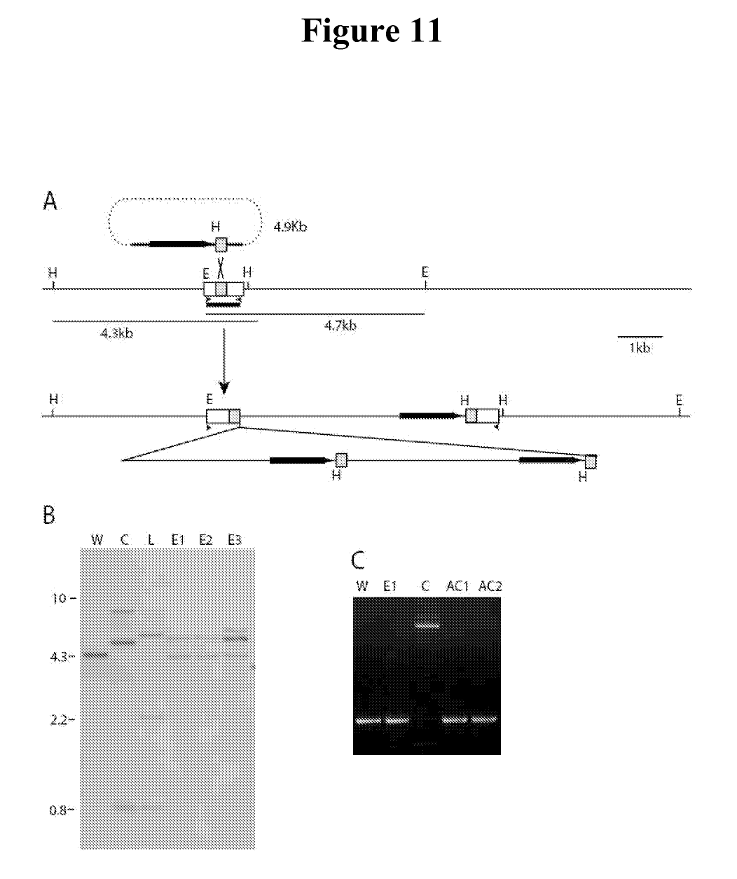

Generation Of amk1 Gene Disruption Mutants And Reintroduction Of Amk1 Gene To The Mutant: A gene encoding MAP kinase in A. brassicicola (Amk1) was initially identified in a subtracted cDNA library derived from infected cabbage and its full length cDNA sequence was determined (Cramer et al., 2006). According to phylogenetic analysis with known kinases in fungi, the gene belonged to the YERK1 kinase subfamily (Xu, 2000). Twenty A. brassicicola transformants were initially generated using a linear minimal element (LME) disruption construct, containing a partial Amk1 gene and the selectable marker gene, Hygromycin phosphotransferase B. All twenty transformants were confirmed by PCR and Southern hybridization as targeted gene disruption mutants (see above). All of them showed altered morphology that was obviously discernable from the wild type. A circular disruption construct was also utilized and produced 17 transformants. Four transformants showed similarly altered morphology indistinguishable from the gene disruption mutants generated with LME constructs while all other transformants showed unmodified morphology indistinguishable from the wild type. One transformant with modified morphology and three transformants with unmodified morphology were selected for further purification via two rounds of single conidia (or fragmented protoconidia) isolation process among the transformants generated with the circular disruption construct. The transformant with the modified morphology was confirmed as a targeted gene disruption mutant by three tandem integration of the circular constructs (FIG. 11). Meanwhile the other three transformants with unaltered morphology were identified as ectopic insertion mutants (ect-c) in which the circular construct was incorporated at unknown loci (FIG. 11). Based on the circular- and the linear-disruption constructs used to generate transformants, the mutants were assigned amk-c and amk-1, respectively. Both amk-c and amk-1 mutants were stable during pathogenicity analysis as described previously above.

The amk-c mutant was utilized in order to reintroduce the wild type copy of Amk1. A total of 14 transformants were produced from the transformation of wild type Amk1 allele with its native promoter and Nourseothricin-resistant cassette. All transformants simultaneously selected by Hygromycin B and Nourseothricin produced mature conidia, suggesting the complementation of the amk-C mutation by the native gene. Two of the 14 transformants (Amk1-comp1, Amk1-comp2) were further purified by two rounds of single conidia isolation and confirmed the reintroduction of wild type allele of the Amk1 (FIG. 11).

More specifically, FIG. 11 shows disruption of the Alternaria brassicicola Amk1 gene. Shown in Panel A are a circular plasmid disruption construct (dotted line) with Amk1 partial sequence (square, 344-789 bp coding region) and Hygromycin phosphotransferase B (HygB) cassette (arrow), a wild type genomic locus, and a locus disrupted by a circular construct. Solid line (2.1 Kb) in the construct indicates a linear minimal element (LME) gene disruption construct (see above) containing the partial Amk1 gene and HygB cassette. The mutated genomic locus is depicted to show three tandem insertions of the disruption plasmid. Panel B shows targeted gene disruption mutants with the circular construct (C) and the linear construct (L) and three ectopic insertion mutants (E1, E2, and E3), which are shown by Southern blot hybridization. Panel C of the figure shows results of reintroduction of the wild type Amk1 gene into the amk1 mutant. Shown are PCR products amplified with two primers at the 5′ and 3′ end of the coding region marked as arrow heads. Template DNA was extracted from wild type, ectopic insertion mutants (E1), amk1-c (C), and two Amk1-reintroduced transformants (AC1, AC2). Abbreviation: H=HindIII, E=EcoRV. Molecular sizes (Kb) marked at far left are estimated based on 1 Kb ladder (Fisher Scientific, Atlanta, Ga.).

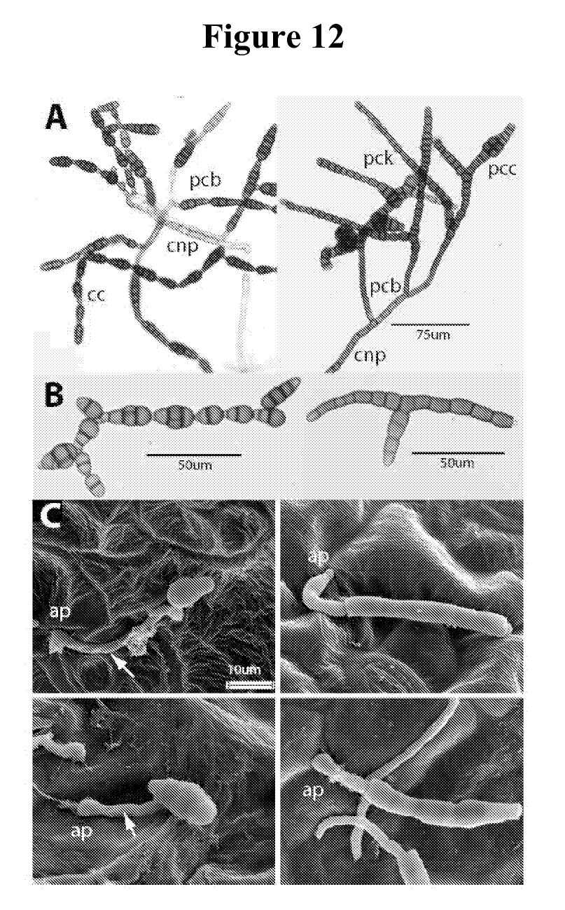

Mutant Morphology: Aerial hyphal growth of wild type A. brassicicola was abundant and consisted almost entirely of short condiophores (9-12×35-75 um) with 1-4 branches (generally 2), each supporting conidial chains 8-18 conidia in length. Each chain supported 1-3 (generally 1) secondary branches 4-14 conidia in length and occasionally a tertiary branch 4-6 conidia in length. Conidia were ovate in shape, melanized, and multicellular with 1-5 transverse and 0-2 longitudinal septa as previously see in the art (see FIG. 12). In contrast, amk1-c mutants showed similar morphology and branching patterns of conidiophores but without fully differentiated conidia. Instead, mutants produced abundant vertically erect, melanized protoconidial chains (9-12 um diameter) with no fully differentiated conidia. The length (130×250 um) and pigmentation of the protoconidial chains were similar to wild type conidial chains (FIG. 12). Occasionally, irregular cell division within the protoconidial chain resulted in the formation of intercalary protoconidial knots. Fully differentiated conidia were never developed by the mutant on PDA, weak PDA ( 1/20 concentration), and GYEA plates under light and dark conditions. Highly similar observations were also made with amk1-1 mutants, while both Amk1-comp1 and Amk1-comp2 showed indistinguishable morphology from wild type (data not shown), supporting that Amk1 is responsible for the morphological alteration of the mutant.

More specifically, FIG. 12 shows the effects of targeted disruption of the Amk1 locus on fungal morphology and virulence. The left panel photos represent typical structures associated with wild type while the right panel photos depict altered structures of amk1-c mutants. Panel A shows terminal structures leading to conidia production. Panel B shows a conidial chain of wild type (left) and a protoconidial chain of amk1-c (right). Panel C shows the ultrastructural interface between host plant and fungi during early infection. Both wild type conidia (left) and amk1-c protoconidial branch fragments (right) germinated to produce one germ tube (arrow) and terminally differentiated into appressorium-like structures on the host plants, green cabbage (Brassica oleracea) (top) and broadleaf mustard (Brassica juncea) (bottom). Abbreviations: cnp=conidiophore, pcb=protoconidial branch, cc=conidial chain, pck=protoconidial knot, ap=appressorium, amk1-c; amk1 disruption mutant generated with a circular plasmid constructs.

Ultrastructural Interface Between Host Plant And Pathogen: The growth patterns of amk-c and wild type were compared at early infection stages on the leaf surface of host plants using SEM. Germination of wild type conidia began approximately eight hours after inoculation on leaves with germ tube emergence occurring typically from one compartment of an individual conidium. Terminal ends of germ tubes differentiated into an appresorium-like structure often referred to as a pseuodopodium (FIG. 12C left panel). The tissues of the host plant typically collapsed beneath the fully differentiated appresorium-like structure. Vegetative growth of amk-c was initiated approximately 18-32 hours after the inoculation, which was 12-24 hours of delay compared to the wild type germination. A single vegetative hypha emerged from one end of fragmented protoconidia both on PDA and on host plant tissue (FIG. 12C right panel). This process was similar to the germination of the wild type in its hypha morphology and its emergence of a germ tube-like structure at one side of the protoconidia. We regarded the emergence of hypha from the protoconidia as germination although the protoconidia are not fully differentiated conidia. The mutants often produced a slightly swollen appresorium-like structure at the tip of the germ tube, however, these appeared elongated and immature compared to the wild type structures. Noticeably, amk-c did not appear to penetrate leaf surface or cause tissue destruction and continued to grow on the leaf surface.

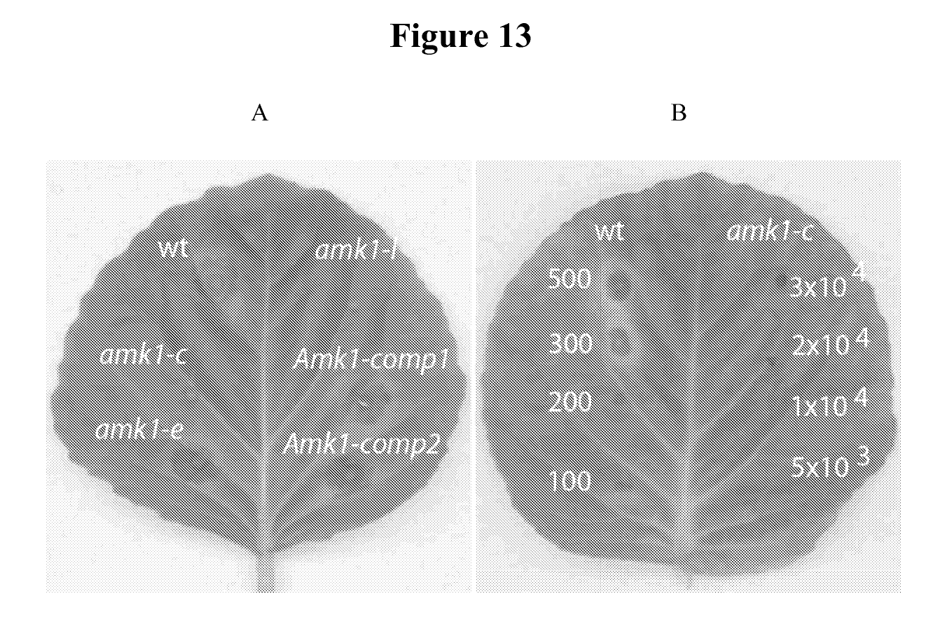

Null Pathogenicity Of amk1 Disruption Mutants: The virulence of wild type, amk1-c, amk1-1, two ectopic insertion mutants (ect-c1, ect-c2), and two complements (comp1, comp2) were compared on detached leaves of host plants, including the Brassica species, B. oleracea, B. napus, B. juncea, and B. carinata. Plant inoculation with either conidia or GYEB grown fragmented mycelia of wild type and ectopic insertion mutants resulted in microscopic dark spots within 24 hours. As few as 100 conidia or mycelial fragments in 10 ul water were sufficient to cause typical blackspot symptoms consisting of dark necrotic areas surrounded by chlorotic halos (see FIG. 13). During late stages of infection with wild type necrotic spots were filled with a hyphal network and exhibited a dense formation of conidia on the surface. In contrast, neither amk1-c nor amk1-1 mutants caused noticeable disease symptoms on host leaves even when inoculations were performed with up to 300-fold more fragmented protoconidia compared to wild type. Two ectopic insertion mutants and Amk1-comp1 and Amk1-com2 caused typical disease symptoms and lesion size was statistically indistinguishable from wild type lesions (FIG. 13). GYEB grown amk1 mutant mycelial fragments also failed to cause disease symptoms in the same range of concentration while wild type mycelia were highly virulent, implying that lack of infection ability is not due to delayed conidial germination (see below). Primary microscopic lesions very rarely developed following inoculation with amk1-c or amk1-1, but never expanded further.

Looking at FIG. 13 in more detail, the figure shows loss of infection ability in two amk1 mutants on two Brassica species B. oleracea. Shown in the left panel are typical infection results with a 10 ul drop containing 1×10⁵/ml conidia for the wild type (wt), an ectopic insertion mutant (amk1-e), and two Amk1-reintroduced transformants (Amk1-comp). An equivalent amount of protoconidia was used for amk1-c and amk1-1 mutants. Images in the right panel depict infection results with various concentrations of conidia (wt) and protoconidia (amk1-c) on the host plant leaves. Black spots with a chlorotic halo surrounding infection loci of wild type conidia are indicative of penetration and symptom formation while the black spots observed at amk1-c inoculation sites are the large number of highly melanized protoconidia initially inoculated. Photos were taken 5 days after inoculation. Numbers indicate estimated amounts of (proto)conidia in 10 ul inoculum.

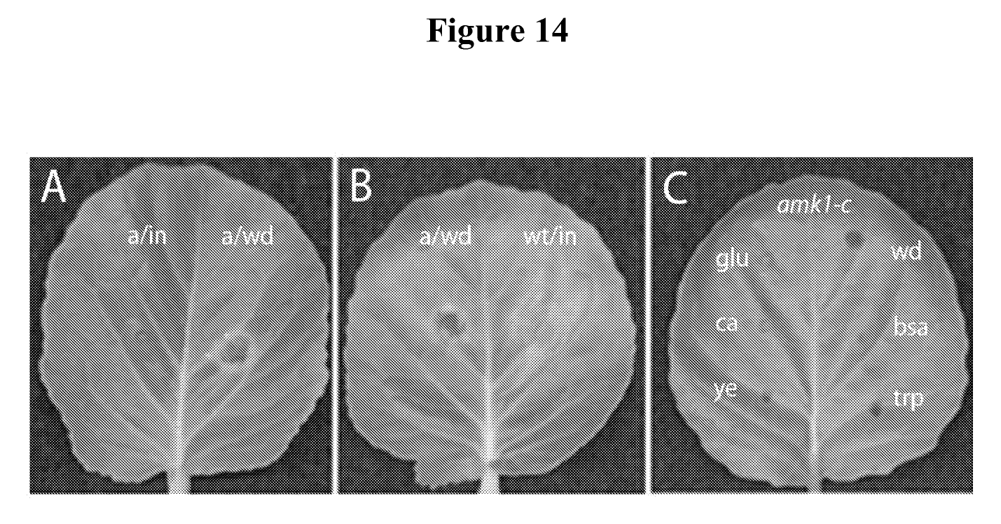

Restoration Of Pathogenicity By Wounding And Nutrient Supplementation: In contrast to the lack of disease symptom development on intact leaves, the mutants were able to partially develop disease symptoms on wounded leaves (FIG. 14). Surprisingly, the mutants also caused disease symptoms on intact leaves, albeit slow, when they were inoculated together with crushed host plant leaves, suggesting importance of host-derived biochemical signals in inducing pathogenicity mechanisms in amk1 mutants (data not shown). Subsequently, several types of chemicals and nutritional supplements were tested for their ability to restore virulence of amk1 mutants. Tryptone supplementation resulted in the most consistent lesion development although it was less efficient when compared to wounding leaves prior to inoculation. Yeast extract and bovine serum albumin also minimally restored infection ability, but neither casamino acids nor glucose supplementation resulted in disease symptom development by the various amk1 mutants (FIG. 14). Amino acid composition is very similar between tryptone and casamino acids because they are derived from the same source (caseine) but digested with trypsin alone or multiple peptidases, respectively. Restoration by tryptone and bovine serum albumin but not casamino acids suggested that long polypeptides are important determinant of the virulence restoration. None of the nutrients caused noticeable toxic effects such as tissue death of host plants. Additionally, neither mutant produced conidia during colonization under test conditions with nutrient supplementation or on wounded tissues. Since mutants showed delayed germination, slower growth, and lack of conidiation, compared to wild type and ectopic insertion mutants, these properties were examined on solid media containing the various supplements.

More specifically with regard to FIG. 14, the figure shows partial restoration of plant pathogenicity of an amk1 mutant. Shown are B. oleracea leaves three days following inoculation with amk1-c mutant or wild type Alternaria brassicicola. The pictures depict the partial restoration of amk1-c virulence due to host plant wounding (Panels A and B) and nutrient supplementation (Panel C). Abbreviations: a/in=amk1-c mutant infected on intact surface, a/wd=amk1-c infected on wounded surface, wt/in=wild type infected on intact surface, glu=glucose, ca=casamino acids, ye=yeast extract, trp=tryptone, bsa=bovine serum albumin.

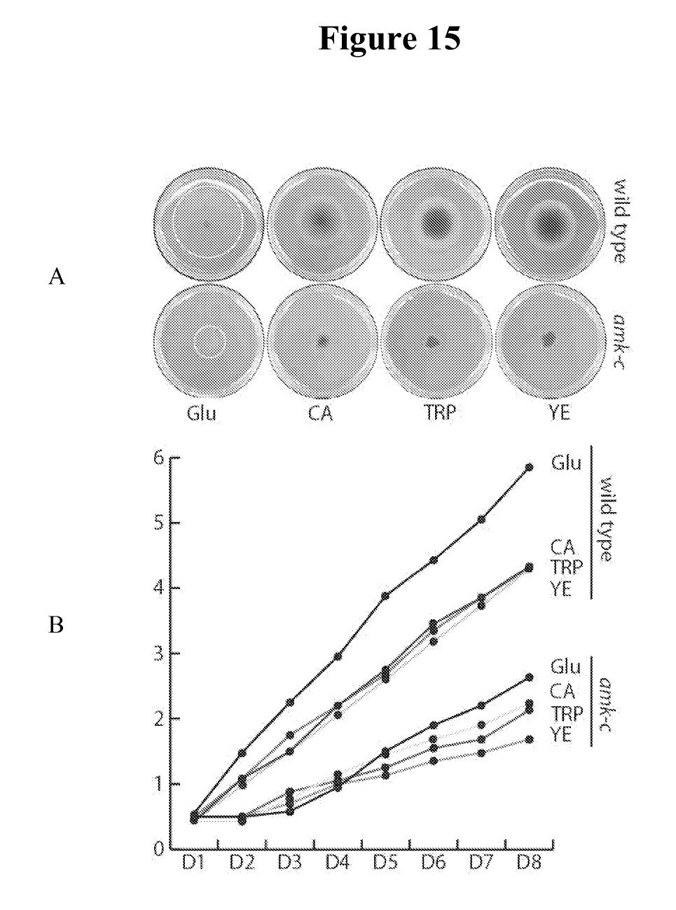

Growth Comparisons With Nutrient Supplements: Conidia of the wild type and ectopic insertion mutants germinated approximately eight hours after inoculation on water agar plates with various nutrients. The germination of mutant protoconidia was delayed approximately 12-24 hours, compared to wild type and ectopic mutant spores. In addition, the mutants grew significantly slower than wild type and ectopic insertion mutants (FIG. 15). However, the differences in growth pattern and rate within each test group were negligible on agar plates with yeast extract, tryptone, or casamino acids. None of the media types noticeably changed the extent of melanin production nor supported mature conidia production in amk-c and amk-1 mutants. The results of these growth comparisons in vitro suggested that changes neither in growth rate, conidiation, nor melanin deposit due to nutrient supplementation were the primary causes for the partial pathogenicity restoration in amk1 mutants.

In summary, FIG. 15 shows the effects of nutrients on the in vitro growth of wild type and amk1-c mutants. Pictures were taken eight days after inoculation on each plate. Boundaries of fungal hyphae were artificially marked with white circles on glucose plates. The chart shows growth rates of fungal colonies measured in diameter (cm) during eight days after inoculation. The standard deviations were so small that they were considered negligible. Ectopic insertion mutants and amk1-1 showed similar patterns to wild type and amk1-c, respectively.

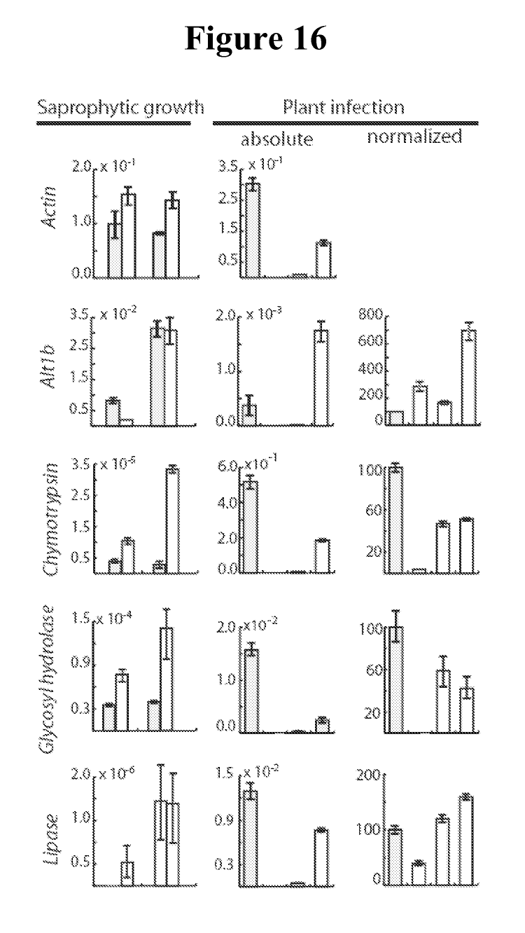

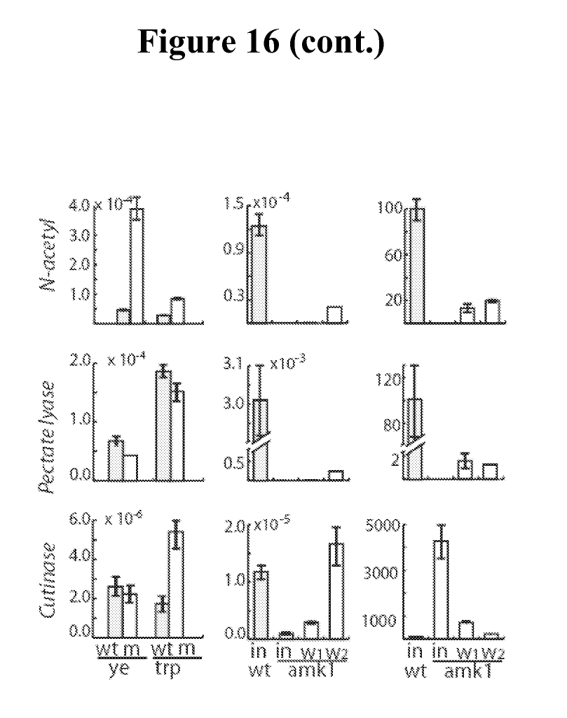

Regulation Of Gene Expression For Hydrolytic Enzymes: We investigated expression patterns of 11 genes in the wild type and in amk1-c mutants during saprophytic growth in nutrient rich media and during host plant infection. These genes were initially identified in EST collections derived from infected B. oleracea and were selected for gene expression surveys based on their predicted functions that were relevant to a hypothesized host pathogen interaction. Six are predicted to encode hydrolytic enzymes while others encode three toxic proteins, an unknown transcription factor, and an ABC transporter. According to the initial semi-quantitative RT-PCR, subset of hydrolytic enzyme genes appeared to be differentially regulated during plant infection and saprophytic growth by Amk1 (data not shown). Gene expression patterns were examined using quantitative RT-PCR for all six hydrolytic enzyme genes, together with a putative toxic allergenic protein gene Alt1b and Actin. The amounts of Actin transcript in the wild type were 0.1 pg in 5 ng total RNA representing 0.1% of mRNA during saprophytic growth in nutrient rich media (see FIG. 16 and Table 4, leftmost column). The expression level of Alt1b was also high, representing ˜0.01 pg in 5 ng total RNA. The expression level for these two genes was similar between the wild type and the amk1 mutant in the culture media. In contrast, all six hydrolytic enzyme genes were expressed at extremely low levels, representing less than 1×10⁻⁴ pg in 5 ng total RNA for each gene in the wild type during saprophytic growth. In addition, Chymotrypsin, Glycosyl hydrolase, and N-acetylglucosaminidase genes showed elevated expression in the mutant compared to the wild type in the nutrient rich media, suggesting negative regulation by Amk1 during saprophytic growth under nutrient rich conditions.

The amounts of Actin transcript were increased to 0.3 pg in 5 ng of total RNA during plant infection compared to the saprophytic growth. The transcripts of all six hydrolytic enzyme genes increased from moderate to dramatic levels in the wild type during plant infection (FIG. 16 and Table 4 middle column). Especially, transcripts for three genes (Chymotrypsin, Glycosyl hydrolase, and Lipase) increased over 1,000-fold compared to saprophytic growth. As a dramatic example, the amount of Chymotrypsin transcripts increased to 0.5 pg in 5 ng total RNA during plant infection from less than 4×10⁻⁶ pg during saprophytic growth in yeast extract and tryptone media. The fungal RNA was estimated to be about 1/10˜ 1/20 of the total RNA based on the Alt1b expression. Considering the proportion ( 1/10) of fungal RNA in the mixed RNA, the Actin transcripts in plant infection samples was 0.3 pg in 0.5 ng of fungal total RNA, representing a 30-fold increase during plant infection. Meanwhile, the Chymotrypsin transcripts represent approximately 0.5 pg in 0.5 ng of fungal total RNA and conservatively a 100,000-fold, possibly 3×10⁶-fold, induction compared to saprophytic growth in culture media. Interestingly, these three genes were highly expressed in the amk1 mutants on wounded host plants but not on intact ones, which was consistent with the pathogenicity restoration. The amounts of transcripts for these three genes (Chymotrypsin, Glycosyl hydrolase, and Lipase) were further examined and confirmed that the expression level was still low even after 5 days post inoculation on intact plants, excluding a possibility of delayed germination and slow growth as a primary source of the low expression (data not shown).

Although the expression level for the other three hydrolytic enzyme genes (N-actetyl glucosaminidase, Pectate lyase, Cutinase) appear to be somewhat increased in the wild type during host infection and restored in the mutants inoculated on wounded host tissues, these increases were indecisive because their expression level was low and there was some degree of uncertainty in the relative amount of fungal RNA, as well as the role of Amk1 in the transcription induction of Actin. In addition, a saprophytic growth specific Cutinase gene was expressed at an extremely low level during plant infection and its absolute expression level was negligible (3.4×10⁻⁷ pg in 5 ng total RNA) even if its expression level was 500-fold higher in amk1 mutants than in the wild type on plant tissues. In contrast, the dramatic increase of the transcripts of the other three genes (Chymotrypsin, Glycosyl hydrolase, and Lipase) was indisputable in the wild type during plant infection and in the amk1 mutants on the wounded host plants. The amount of transcripts expressed by amk1 mutants on wounded host plants is comparable to the wild type during disease symptom development (FIG. 16). For example, transcripts for the Chymotrypsin gene represented ˜50% of the wild type (2.5% of fungal mRNA) in amk1-c at 53 hours after infection and similar amount to wild type (5%) of fungal mRNA at 72 hours after infection.

With regard to FIG. 16, the figure depicts results of experiments that show the role of Amk1 in the regulation of hydrolytic enzyme gene expression. Shown are quantitative RT-PCR results for the expression of Actin, Alt1b, and six hydrolytic enzyme genes. The charts in the left two columns depict absolute amount (pg) of transcripts in 5 ng of total RNA extracted from fugal tissues grown in culture media (left) and from mixed tissues of fungi and host plants (middle) inoculated with wild type (53 hours), amk1-c on intact plant leaves (53 hours), and amk1-c on wounded plant leaves (w1=53 hours and w2=90 hours). The charts on the right show the relative amount of transcripts compared to wild type (100%) after normalization of fungal RNA using fungal specific Actin gene transcript. The error bars indicate standard deviations from three technical replicates for one of two biological replicates. All shaded bars and open bars in the charts represent wild type and amk1-c, respectively. Abbreviations, in=intact leaf, w1=wounded tissue infected for 53 hour infection, w2=wounded tissue infected for 90 hours, ye=1% yeast extract, trp=1% tryptone.

TABLE 4
Statistics Of Quantitative RT-PCR Results

| | Saprophytic | | Infection Before | | | Infection After | |
|---|---|---|---|---|---|---|---|
| | Growth | | Normalization | | | Normalization | |
| Sample Name | Mean** | s.d. | Sample name* | Mean** | s.d. | Mean# | s.d. |
| Actin | | | | | | | |
| wt/yeast extract | 9.90E−02 | 4.98E−02 | wt/Intact | 3.03E−01 | 4.45E−02 | | |
| amk1/yeast extract | 1.54E−01 | 3.20E−02 | amk1/Intact | 6.82E−04 | 1.13E−04 | | |
| wt/tryptone | 8.26E−02 | 3.98E−03 | amk1/wound | 9.82E−03 | 8.37E−04 | | |
| amk1/tryptone | 1.43E−01 | 3.01E−02 | amk1/wound2 | 1.12E−01 | 1.18E−02 | | |
| Alt1b | | | | | | | |
| wt/yeast extract | 8.25E−03 | 1.81E−03 | wt/Intact | 3.77E−04 | 3.85E−04 | 2.07E−04 | 2.91E−05 |
| amk1/yeast extract | 2.08E−03 | 5.72E−04 | amk1/Intact | 2.74E−06 | 1.27E−06 | 5.93E−04 | 1.53E−04 |
| wt/tryptone | 3.16E−02 | 5.28E−03 | amk1/wound | 2.36E−05 | 3.26E−06 | 3.48E−04 | 4.77E−05 |
| amk1/tryptone | 3.09E−02 | 8.85E−03 | amk1/wound2 | 1.75E−03 | 3.77E−04 | 1.44E−03 | 2.74E−04 |
| Chymotrypsin | | | | | | | |
| wt/yeast extract | 3.93E−06 | 1.81E−06 | wt/Intact | 5.19E−01 | 7.46E−02 | 1.73E+00 | 2.74E−01 |
| amk1/yeast extract | 1.04E−05 | 2.71E−06 | amk1/Intact | 2.80E−05 | 2.93E−06 | 4.24E−02 | 1.17E−02 |
| wt/tryptone | 4.22E−06 | 2.82E−06 | amk1/wound | 7.82E−03 | 9.29E−04 | 7.99E−01 | 9.66E−02 |
| amk1/tryptone | 3.33E−05 | 3.33E−06 | amk1/wound2 | 1.86E−01 | 9.07E−03 | 1.66E+00 | 1.42E−01 |
| Glycosyl hydrolase | | | | | | | |
| wt/yeast extract | 3.53E−05 | 6.1E−06 | wt/Intact | 1.57E−02 | 2.35E−03 | 5.33E−02 | 1.55E−02 |
| amk1/yeast extract | 7.74E−05 | 2.23E−05 | amk1/Intact | 0.00E+00 | 0.00E+00 | 0.00E+00 | 0.00E+00 |
| wt/tryptone | 3.97E−05 | 8.24E−06 | amk1/wound | 3.06E−04 | 1.56E−04 | 3.12E−02 | 1.52E−02 |
| amk1/tryptone | 1.40E−04 | 8.68E−05 | amk1/wound2 | 2.44E−03 | 9.06E−04 | 2.24E−02 | 1.08E−02 |
| Lipase | | | | | | | |
| wt/yeast extract | UD | UD | wt/Intact | 1.29E−02 | 2.20E−03 | 4.29E−02 | 6.07E−03 |
| amk1/yeast extract | UD | UD | amk1/Intact | 1.13E−05 | 1.27E−06 | 1.68E−02 | 3.19E−03 |
| wt/tryptone | 2.90E−06 | 1.91E−06 | amk1/wound | 5.02E−04 | 3.04E−05 | 5.14E−02 | 5.88E−03 |
| amk1/tryptone | UD | UD | amk1/wound2 | 7.64E−03 | 5.25E−04 | 6.83E−02 | 3.94E−03 |
| N-acetyl | | | | | | | |
| glucosaminidase | | | | | | | |
| wt/yeast extract | 4.68E−05 | 8.29E−06 | wt/Intact | 1.24E−04 | 2.81E−05 | 4.20E−04 | 1.26E−04 |
| amk1/yeast extract | 3.88E−04 | 9.87E−05 | amk1/Intact | 2.41E−07 | 1.20E−07 | 3.45E−04 | 1.49E−04 |
| wt/tryptone | 2.83E−05 | 2.98E−06 | amk1/wound | 1.03E−06 | 8.52E−07 | 1.09E−04 | 9.24E−05 |
| amk1/tryptone | 8.49E−05 | 9.80E−06 | amk1/wound2 | 2.05E−05 | 5.03E−07 | 1.85E−04 | 2.44E−05 |
| Pectate lyase | | | | | | | |
| wt/yeast extract | 6.78E−05 | 1.42E−05 | wt/Intact | 2.92E−03 | 1.87E−04 | 1.50E−03 | 9.36E−05 |
| amk1/yeast extract | 4.32E−05 | 2.88E−06 | amk1/Intact | UD | UD | UD | UD |
| wt/tryptone | 1.86E−04 | 1.99E−05 | amk1/wound | 1.67E−06 | 1.21E−06 | 2.46E−05 | 1.78E−05 |
| amk1/tryptone | 1.52E−04 | 2.87E−05 | amk1/wound2 | 2.43E−05 | 6.64E−07 | 1.93E−05 | 5.22E−07 |
| Cutinase | | | | | | | |
| wt/yeast extract | 2.61E−06 | 9.21E−07 | wt/Intact | 1.18E−05 | 2.50E+06 | 6.06E−06 | 1.27E−06 |
| amk1/yeast extract | 2.24E−06 | 8.68E−07 | amk1/Intact | 9.93E−07 | 3.43E−07 | 2.58E−04 | 8.94E−05 |
| wt/tryptone | 1.75E−06 | 7.47E−07 | amk1/wound | 2.77E−06 | 3.65E−07 | 4.36E−05 | 5.36E−06 |
| amk1/tryptone | 5.42E−06 | 1.45E−06 | amk1/wound2 | 1.66E−05 | 6.77E−06 | 1.32E−05 | 5.40E−06 |

UD stands for undetectable in this study
*Total RNA was extracted from the mixed tissues of plant and fungi 53 hours post inoculation for the first three samples and 90 hours post inoculation for the amk1 wound2.
**Indicates the absolute amounts (pg) of transcript in 5 ul total RNA.
#Indicates the arbitrary unit of transcripts normalized by fungal Actin transcripts.

Discussion of Results Obtained in Example 3:

The Amk1 MAP kinase described in this study belongs to the yeast and fungal extracellular signal-regulated kinase (YERK1) subfamily of the MAP kinase superfamily. YERK1 in S. cereviseae (Fus3) and S. pombe (Spk1) is known to be activated by pheromones and mating signals for cell cycle regulation and conjugation. Members of the YERK1 subfamily are also known to be required for virulence in most filamentous phytopathogenic fungi studied. These genes are involved in several processes including conidiation, germination, appressoria formation, initial penetration to the host, and melanin production with exceptions in every category depending upon the organism under investigation.

The altered traits of amk1 mutants include incomplete conidiation, incomplete appressorium development, delayed germination, slow vegetative growth, and near complete loss of pathogenicity. The mutant phenotypes show similarity to the MAP kinase mutants of most phytopathogenic fungi, including closely related Dothideomycete fungi S. nodorum, P. teres, and C. heterostrophus with three distinctive differences. First, there is no obvious sign of autolysis of hyphae on the mutant colonies in contrast to a delta chk1 mutant. Second, the amk1 mutants form incomplete or immature appressoria-like structure at the tip of germ tubes. Third and most distinctively, they are able to colonize wounded host tissues in contrast to the MAP kinase mutants in diverse phytopathogenic fungi, including M. grisea, C. lagenarium, B. cinerea, C. purpurea, and a closely related fungus P. teres. The latter two features are similar to the mutants of YERK2 subfamily mps1 and cmk1 that are involved in early appressorium formation in M. grise and appressorium maturation in C. lagenarium. There is a homolog of YERK2 subfamily in A. brassicicola genome and gene disruption mutants were generated (unpublished data). Mutant phenotypes and downstream genes controlled by the gene are yet to be investigated. The amk1 disruption mutant is distinctive from the YERK1 type MAP kinase mutants in other filamentous phytopathogenic fungi in that it maintains colonization ability and pathogenicity is partially restored when inoculations are supplemented with polypeptide rich nutrients. The loss and the restoration of pathogenicity in amk1 disruption mutants correlate with expression modulation of putative CWDE genes. It is known that a G-protein mutant with reduced appressorium formation was able to penetrate and colonize as wild type in C. heterostrophus, indirectly supporting the importance of hydrolytic enzymes. Among YERK1 MAP kinase null mutants, there are two examples showing the association of CWDE gene expression to pathogenicity. Unlike other fungi that lose pathogenicity by MAP kinase mutation, C. parasitica MAP kinase mutants are pathogenic and express hydrolytic enzymes. In addition, T. virens mutants that enhance mycoparasitic properties have been shown to transcribe an even higher level of CWDE genes compared to wild type.

The accumulation of mycotoxin in vitro was not affected in amk1 mutant (unpublished data), resembling F. graminiarum. Increased expression of a Cutinase gene in amk1 mutant was unexpected, however, the absolute amounts of the transcript were still very low. It has been shown that this gene is saprophytic growth specific and not a pathogenicity factor in A. brassicicola. Three of six genes encoding putative CWDE are induced during plant infection over a 1,000-fold and a dramatic example of over 100,000-fold increase of transcripts for a Chymotrypsin gene. The transcripts for these genes are extremely low in amk1-c mutants on host plants, supporting the notion that Amk1 is essential for transcriptional activation. Meanwhile, the expression level of these genes is similarly low in the wild type during saphrophytic growth in culture media. In contrast, it is elevated in amk1 disruption mutants during saprophytic growth compared to the wild types (FIG. 16), implying negative regulation by Amk1 under these conditions. The increased level of transcripts in the mutants, however, results in less than 1×10⁻⁴ pg in 5 ng total RNA and this result does not contradict the unchanged proteolytic activities in the mutants of the S. nodorum homolog. Furthermore, elicitors, such as cell wall components of host plants, might be required for the full induction of putative CWDE gene expression as shown in T. virens mutants. N-acetylglucosaminidase and Cutinase provide additional examples of negative regulation. There are relatively few examples of genes whose expression is regulated by MAP kinases either positively during plant infection in other phytopathogens, or negatively with little implication of the function in nature. These studies with A. brassicicola provide an unprecedented example of dual functions of a MAP kinase to regulate the gene expression of CWDE with an explosive induction of these genes during plant infection and a near absolute repression during saprophytic growth under nutrient rich conditions. We speculate that Amk1 serves as a control point limiting the unnecessary expression of CWDE genes in nutrient rich environments and positively regulating expression of genes necessary to break down complex substrates such as plant material in nutrient limiting environments.

The amk1 disruption mutants nearly lost pathogenicity primarily due to the loss of penetration ability. Possibly, the mutants can colonize the wounded host plants because they simply bypass the cell wall barriers. Alternatively, unknown components from wounded or partially disrupted plant tissue altered the mutant's physiology in such a manner as to be able to colonize host plants despite the delayed germination and slow vegetative growth. Supporting this hypothesis, three genes encoding putative CWDE are dramatically activated during colonization of wounded host plants compared to wild type during disease symptom development suggesting that wounded plant tissue contains signals such as cell wall hydrolysis products inducing these genes. These genes are also slightly induced during growth in the polypeptide rich media that partially restored the infection ability of the mutant. The elevated expression by MAP kinase mutants of these genes in nitrogen rich media is an unprecedented example among filamentous fungi. The nitrogen rich nutrients that support partial restoration of pathogenicity include tryptone, peptone, yeast extract, and bovine serum albumin. Interestingly, the pathogenicity is never restored by casamino acids that are comprised mainly of amino acids derived from the same origin (casein) of the tryptone. Our results suggest the presence of an additional pathway to moderately activate these genes and that the pathway can be suppressed by the MAP kinase pathway. Together with the characterization of the promoter elements of the Chymotrypsin gene, the mode of transcriptional regulation would provide clues to elucidate the evolutionary perspective of acquisition of the control mechanism of gene expression via this MAP kinase and possibly pathogenicity acquisition.

### Example 4

### Use of an LME to Engineer a Histidine Tag into a Protein

The LME technology of the invention was used to engineer a histidine tag on the C-terminus of an Alternaria protein. The protein was subsequently over-expressed in the host cell, affinity purified using the engineered histidine tag, and detected on protein gels and Western blots. The procedure described for this particular protein is applicable to any protein for which sequence information is available.

In general, the procedure comprises designing an LME construct having sequence in which a portion is homologous to a gene of interest at the region encoding the C-terminus of a protein. The LME comprises, in frame with the protein coding region, a region that codes for multiple (e.g., six) contiguous histidine residues. It is known that such a “histidine tag” or “his tag” is capable of binding Nickel, and thus can be used to purify proteins containing the tag. The LME further comprises a detectable marker, such as an antibiotic resistance gene.

The LME is exposed to the host genome under conditions that allow for homologous recombination. Resulting recombinant cells comprising the LME or a portion of it can be grown in culture. The cells can then be assayed for expression of the protein of interest, preferably after purification of the protein by way of Ni-affinity purification. The purified protein can be detected in many ways, such as by simple Coomassie blue staining, silver staining, or Western blotting (using an antibody that is specific for the protein or for the His tag). The purified protein can be used in any number of applications, including research on the protein, per se, or use of the protein in research or clinical (e.g., diagnostics, therapeutics) settings. The genes that can be targeted are not limited; however, to minimize the number of targeted recombination events needed, it is preferred that they be those that are constitutively expressed. The proteins can be cytoplasmic, membrane-bound, or secreted. Where the protein is cytoplasmic, cell lysis can be used to obtain the protein. Where the protein is membrane-bound, solubilization techniques known in the art can be used to free it from the membrane. Secreted proteins may be collected from growth media supporting growth of the recombinant cells.

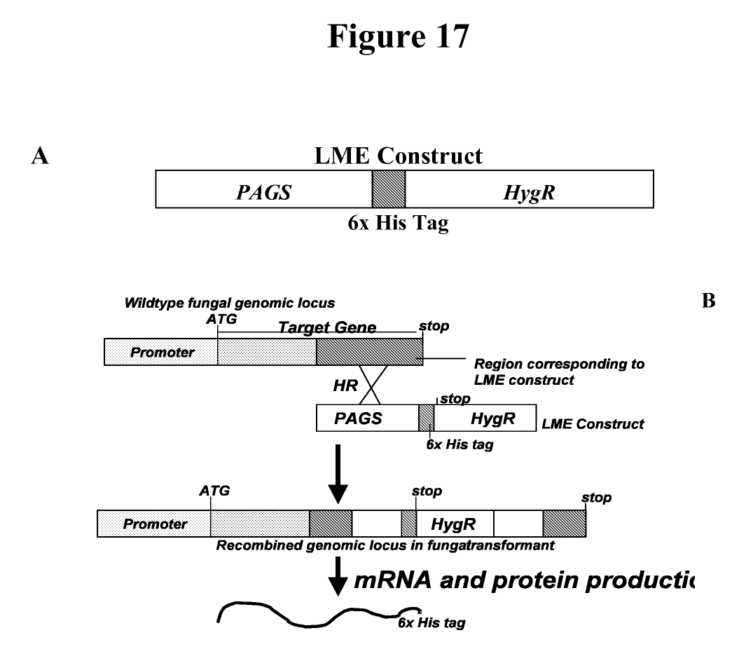

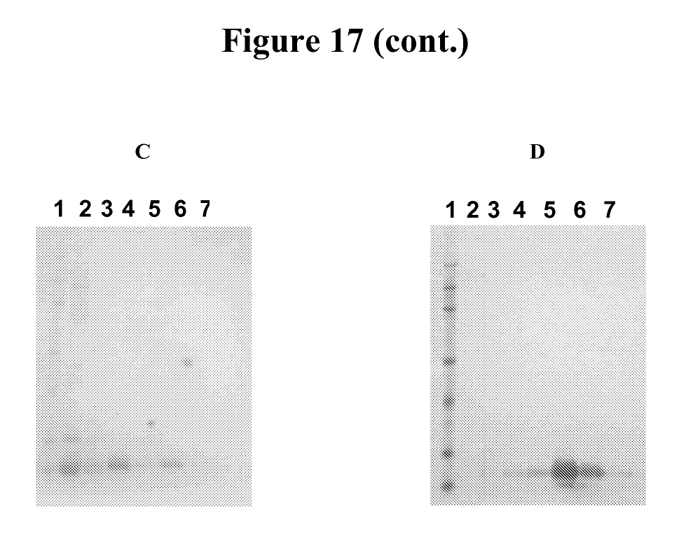

FIG. 17 shows an example of the technology. In FIG. 17, production of a recombinant cell and constitutive expression of a secreted recombinant Alternaria protein is shown. More specifically, Panel A shows an LME construct for recombinant protein production comprising a partial Alternaria gene sequence (PAGS) linked to a his tag comprising coding sequence for 6 contiguous histidine residues (6× His tag). The LME construct also contains a selectable marker, which is a hygromycin resistance gene (HygR). The PAGS sequence corresponds to approximately 300-500 bp of the 3′ end of the coding region of an A. brassicicola gene with unknown function referred to hereafter as Alt_{—}211 (SEQ ID NO:37) constitutively expressed or inducible in vitro. Using publicly available signal peptide prediction software (Signal P 3.0) it was determined that the hypothetical protein encoded by the Alt_{—}211 gene contains a predicted signal peptide sequence for extracellular secretion and thus mature protein should be secreted into the media if the fungus is grown under suitable in vitro conditions. A commonly employed PCR technique called “overlap PCR” was used to amplify and mutagenize the region by incorporating a 6× His tag coding sequence just prior to the translational stop codon and attaching the antibiotic resistance conferring cassette (hygromycin resistance) to form a single continuous linear double stranded DNA suitable for fungal transformation and selection of hygromycin-resistant mutants. This new PCR amplified Alt_{—}211_PAGS-6×His-HygR construct was then used for fungal transformation and selection of mutants suitable for protein production. Panel B depicts the mechanism of recombination and fungal transformation between the LME construct and genomic locus.

FIG. 17, Panels C and D, show the results of expression of the recombinant protein. Panel A) shows an SDS PAGE gel of A. brassicicola proteins secreted in glucose yeast extract media at 8 days of growth and then fractionated using Ni-column chromatography according to standard protocols. Panel B shows a Western blot analysis of proteins separated on an identical gel as in Panel A, which were then transferred to a nylon membrane and then probed with anti-H is tag antibodies. The Western blot shows a strong hybridization signal in lane 6, which is the first elution fraction off the column. Significant protein amounts are also detected in this lane in the SDS-PAGE gel as well. Lane loading for Panels A and B is as follows, lane 1—molecular weight marker; lane 2—column flow through; lane 3—first column wash; lane 4—second column wash; lane 5—third column wash; lane 6—first elution fraction; lane 7—second elution fraction; lane 8—third elution fraction. The results of the two gels indicate that the protein of interest was greater than 95% pure. Milligram quantities of protein was obtained from 100 ml of culture media in this experiment.

It will be apparent to those skilled in the art that various modifications and variations can be made in the practice of the present invention and in construction of the nucleic acids without departing from the scope or spirit of the invention. Other embodiments of the invention will be apparent to those skilled in the art from consideration of the specification and practice of the invention. It is intended that the specification and examples be considered as exemplary only, with a true scope and spirit of the invention being indicated by the following claims.

SEQUENCE LISTING

| | |
|---|---|
| ACGGGGTACCTTGGAATGCATGGAGGAGT | (SEQ ID NO:1) |
| TTATCTCGAGTTGCGCGCTATATTTTGTTTT | (SEQ ID NO:2) |
| GTGCACTAGTTCATTCTAGCTTGCGGTCCT | (SEQ ID NO:3) |
| ACATCCACGGGACTTGAGAC | (SEQ ID NO:4) |
| GGCTCTCGAGTTTGGATGCTTGGGTAGATAG | (SEQ ID NO:5) |
| TTGCTAAGCTTGGCTATATTCATTCATTGTCAGC | (SEQ ID NO:6) |
| ACAACTCGAGCAGCAATGCGCGATTTCATA | (SEQ ID NO:7) |
| AGGTGGGCCCTACGCCGTCTGGTTCAAATAC | (SEQ ID NO:8) |
| TAGGGCCCATGGTGAGCAAGGGCGAGGA | (SEQ ID NO:9) |
| ACAAGCTTTGGTTCCCGGTCGGCATCTA | (SEQ ID NO:10) |
| GAGGGCCCCGTGGGCCGTGTGTGGTTTC | (SEQ ID NO:11) |
| GTGGTACCCAGCCTCGCAGACACTCGAC | (SEQ ID NO:12) |
| CTTGAAGCTTTCCTTCCTGCTGTCGATGTT | (SEQ ID NO:13) |
| CCATTCTAGAATGCGTCTGGGAATTGGCAC | (SEQ ID NO:14) |
| GCAAGCTTGAGCTTTCGGACGACCATTGC | (SEQ ID NO:15) |
| GTGAATTCCGGCAGCGATCGAATGTATTCG | (SEQ ID NO:16) |
| ACCTTCCCTGTGTTTTGCAC | (SEQ ID NO:17) |
| TGTGTTTGTTTCCAAGAAAAGAGGGCATTGAAGGTG | (SEQ ID NO:18) |
| TAGCAT | |
| ATGCTACACCTTCAATGCCCTCTTTTCTTGGAAACA | (SEQ ID NO:19) |
| AACACA | |
| TCATTCTAGCTTGCGGTCCT | (SEQ ID NO:20) |
| GGCAACATTGTCATGTCTGG | (SEQ ID NO:21) |
| GAGCGAAGCAAGAATGGAAC | (SEQ ID NO:22) |
| ACAACCTCGGCTTCAACATC | (SEQ ID NO:23) |
| AGCGACATAGGTGGTGTCGT | (SEQ ID NO:24) |
| CGGTACCACTGGAAACACT | (SEQ ID NO:25) |
| TGGGTGAGACCAGTAACACG | (SEQ ID NO:26) |
| GTGGCATGCAATATGGACAA | (SEQ ID NO:27) |
| GCGGTAGAAATTTGCCTTGA | (SEQ ID NO:28) |
| CATTCTGGGGACGTTCCAT | (SEQ ID NO:29) |
| CTGTTCGCTCCGAAGTTCAT | (SEQ ID NO:30) |
| ACACAGGGTAGAACCCAAC | (SEQ ID NO:31) |
| GTTTTCCCGCTGTCAAAGAA | (SEQ ID NO:32) |
| CCACTTGACCGTCACCTACA | (SEQ ID NO:33) |
| GTGTTCTTGCTGTTGCCAAA | (SEQ ID NO:34) |
| CGTAGAGAATGCCCTTCCAG | (SEQ ID NO:35) |
| CGACACCCTTGATTTGGTCT | (SEQ ID NO:36) |
| ATGCAGTTCACCACCGCCATCTTCGCTCTCGCTGCT | (SEQ ID NO:37) |
| GCGACTGCCGCCAACGCCGCTTCAACCTACGGTGCC | |
| TTCAACGTCACCGTCGAGAGCTCTAGCTACGCCAAC | |
| GGCGTCACCTCGCGCACCGTCCTCTCCGACTACTCT | |
| GGCGACGCCGCTATCCACGCCGTCTGCAAGTACGAG | |
| TTCAACCCTGCTGCCGAGCCCAAGGAGACCTCCAGC | |
| TGCGAGCCCAACTCTTTCTCCTACGAGTACGATGGC | |
| CAGACCATCAAGGTCCAGCAGATTGTCGAGAAGCCC | |
| AACCCCATGACCGTCTTCGGTGAGGCTCCTCTTGCC | |
| CTCACTACCGAGGGCGGCGCTGGCAGGACCTCCAAG | |
| GGTAACGCCATCTTCGACGCCACCAGTGCCATCGCT | |
| TAA | |

### REFERENCES CITED

All references cited herein are incorporated into this document in their entireties by reference.
Akamatsu, H., Itoh, Y., Kodama, M., Otani, H., and Kohmoto, K. 1997. AAL-toxin-deficient mutants of Alternaria alternata tomato pathotype by restriction enzyme mediated integration. Phytopathology 87:967-972.
Bussink, H. J., and Osmani, S. A. 1999. A mitogen-activated protein kinase (MPKA) is involved in polarized growth in the filamentous fungus, Aspergillus nidulans. FEMS (Fed. Eur. Microbiol. Soc.) Microbiol. Lett. 173:117-125.
Cramer, R. A., La Rota, C. M., Cho, Y., Thon, M., Craven, K. D., Knudson, D. L., Mitchell, T. K., and Lawrence, C. B. 2006. Bioinformatic analysis of expressed sequence tags derived from a compatible Alternaria brassicicola-Brassica oleracea interaction. Mol. Plant Pathol. 7, 113-124.
Lorang, J. M., Tuori, R. P., Martinez, J. P., Sawyer, T. L., Redman, R. S., Rollins, J. A., Wolpert, T. J., Johnson, K. B., Rodriguez, R. J., Dickman, M. B., and Ciuffetti, L. M. 2001. Green fluorescent protein is lighting up fungal biology. App!. Environ. Microbiol. 67:1987-1994.
Malonek, S., Rojas, M. C., Hedden, P., Gaskin, P., Hopkins, P., and Tudzynski, B. 2004. The NADPH-cytochrome P450 reductase gene from Gibberella fujikuroi is essential for gibberellin biosynthesis. J. Biol. Chem. 279, 25075-84.
Martin-Hernandez, A. M., Dufresne, M., Hugouvieux, V., Melton, R., and Osbourn, A. 2000. Effects of targeted replacement of the tomatinase gene on the interaction of Septoria lycopersici with tomato plants. Mol. Plant-Microbe Interact. 13:1301-1311.
Punt, P. J., Oliver, R. P., Dingemanse, M. A., Pouwels, P. H., and Van den Hondel, C. A. 1987. Transformation of Aspergillus based on the hygromycin B resistance marker from Escherichia coli. Gene 56:117-124.
Reynolds, E. S. 1963. The use of lead citrate at high pH as an electron opaque stain in electron microscopy. J. Cell Biol. 17, 208-212.
Schmitt, E. K., Hoff, B., and Kuck, U. 2004. AcFKHI, a novel member of the forkhead family, associates with the RFX transcription factor CPCRI in the cephalosporin C-producing fungus Acremonium chrysogenum. Gene 342:269-281.
Shah-Mahoney, N., Hampton, T., Vidaver, R., and Ratner, D. 1997. Blocking the ends of transforming DNA enhances gene targeting in Dictyostelium. Gene 203:33-41.
Shiotani, H., and Tsuge, T. 1995. Efficient gene targeting in the filamentous fungus Alternaria alternata. Mol. Gen. Genet. 248:142-150.
Sweigard, J. A., Carroll, A. M., Farrall, L., Chumley, F. G., and Valent, B. 1997. A series of vectors for fungal transformation. Fungal Genet. News. 44:52-53.
Tsuge, T., Nishimura, S., and Kobayashi, H. 1990. Efficient integrative transformation of the phytopathogenic fungus Alternaria alternata mediated by the repetitive rDNA sequences. Gene 90:207-214.
Voigt, C. A., Schafer, W., and Salomon, 5. 2005. A secreted lipase of Fusarium graminearum is a virulence factor required for infection of cereals. Plant J. 42:364-375.
Xu, J. R. 2000. Map kinases in fungal pathogens. Fungal Genet. Biol. 31, 137-152.
Yang, L., Ukil, L., Osmani, A., Nahm, F., Davies, J., De Souza, C. P., Dou, X., Perez-Balaguer, A., and Osmani, S. A. 2004. Rapid production of gene replacement constructs and generation of a green fluorescent protein-tagged centromeric marker in Aspergillus nidulans. Prokaryot. Cell 3:1359-1362.
Yao, C., and Koller, W. 1995. Diversity of cutinases from plant pathogenic fungi: different cutinases are expressed during saprophytic and pathogenic stages of Alternaria brassicicola. Mol. Plant-Microbe Interact. 8:122-130.

1. An isolated or purified nucleic acid comprising:
a nucleotide sequence that is homologous to at least a portion of a target gene from a fungus of the genus Alternaria; and
at least one nucleotide sequence having a known sequence, length, or other detectable characteristic.

2. The nucleic acid of claim 1, wherein the nucleotide sequence is homologous to a sequence from Alternaria brassicicola.

3. The nucleic acid of claim 1, further comprising at least one transcription control element operably linked to the nucleotide sequence having a known sequence, length, or other detectable characteristic.

4. The nucleic acid of claim 1, wherein the nucleic acid is a linear minimal element that causes expression of a host genomic gene when the nucleic acid is inserted into the host genome.

5. A method of specifically disrupting a target gene in a fungal cell of the genus Alternaria, said method comprising:
contacting the cell with a nucleic acid comprising i) a nucleotide sequence that is homologous to at least a portion of a target gene from the host cell, and ii) at least one nucleotide sequence having a known sequence, length, or other detectable characteristic; and
subjecting the organism to conditions that permit uptake of the nucleic acid into the cell and integration of some or all of the nucleic acid into the genome of the cell,
wherein integration of the nucleic acid occurs specifically at a site in the genome having a homologous or complementary sequence to a portion of the nucleic acid.

6. The method of claim 5, comprising determining whether the detectable marker is expressed.

7. The method of claim 5, which is a method of reducing or eliminating expression of one or more target genes in the genome of the organism.

8. The method of claim 5, which is a method of over- or hyper-expressing an endogenous gene of the organism.

9. The method of claim 5, wherein the organism is Alternaria brassicicola.

10. The method of claim 5, wherein the nucleic acid is a linear minimal element.

11. A method of producing a protein of interest in an organism of the Alternaria genus, said method comprising:
integrating into the host cell genome, by homologous recombination, a nucleic acid comprising sufficient information to cause expression of the protein of interest upon integration of the nucleic acid into the genome; and
subjecting the organism to conditions that allow expression of the protein of interest.

12. The method of claim 11, wherein the nucleic acid comprises the coding region for the protein of interest.

13. The method of claim 11, wherein the nucleic acid comprises an expression control region that allows for expression of the protein of interest when integrated into the host cell genome.

14. The method of claim 11, wherein the nucleic acid is a linear minimal element.

15. A method of identifying genes encoding products having detectable activities, said method comprising:
generating a library of nucleic acids comprising a sequence encoding a detectable product and a sequence that is homologous to a sequence in the genome of an organism in the Alternaria genus,
wherein the library comprises more than one distinct sequence that is homologous to sequences in the Alternaria species genome, each of said distinct sequences being present on a different nucleic acid of the library;
integrating the nucleic acids of the library into the genomes of host organisms of the Alternaria genus; and
detecting changes in one or more detectable characteristics of the organism.

16. The method of claim 15, wherein the nucleic acid is a linear minimal element.

17. The method of claim 15, wherein the characteristic is growth of the organism, pathogenicity of the organism, reproduction of the organism, or production of a detectable protein.

18. The method of claim 15, wherein the characteristic is production of a detectable toxin.

19. The method of claim 15, wherein the nucleic acids are linear minimal elements.


SEQUENCE LISTING (38 sequences)

SEQ ID NO: 1 - DNA, 29 bp, Artificial Sequence
  Description of Artificial Sequence Synthetic primer
acggggtaccttggaatgcatggaggagt

SEQ ID NO: 2 - DNA, 31 bp, Artificial Sequence
  Description of Artificial Sequence Synthetic primer
ttatctcgagttgcgcgctatattttgtttt

SEQ ID NO: 3 - DNA, 30 bp, Artificial Sequence
  Description of Artificial Sequence Synthetic primer
gtgcactagttcattctagcttgcggtcct

SEQ ID NO: 4 - DNA, 20 bp, Artificial Sequence
  Description of Artificial Sequence Synthetic primer
acatccacgggacttgagac

SEQ ID NO: 5 - DNA, 31 bp, Artificial Sequence
  Description of Artificial Sequence Synthetic primer
ggctctcgagtttggatgcttgggtagatag

SEQ ID NO: 6 - DNA, 34 bp, Artificial Sequence
  Description of Artificial Sequence Synthetic primer
ttgctaagcttggctatattcattcattgtcagc

SEQ ID NO: 7 - DNA, 30 bp, Artificial Sequence
  Description of Artificial Sequence Synthetic primer
acaactcgagcagcaatgcgcgatttcata

SEQ ID NO: 8 - DNA, 31 bp, Artificial Sequence
  Description of Artificial Sequence Synthetic primer
aggtgggccctacgccgtctggttcaaatac

SEQ ID NO: 9 - DNA, 28 bp, Artificial Sequence
  Description of Artificial Sequence Synthetic primer
tagggcccatggtgagcaagggcgagga

SEQ ID NO: 10 - DNA, 28 bp, Artificial Sequence
  Description of Artificial Sequence Synthetic primer
acaagctttggttcccggtcggcatcta

SEQ ID NO: 11 - DNA, 28 bp, Artificial Sequence
  Description of Artificial Sequence Synthetic primer
gagggccccgtgggccgtgtgtggtttc

SEQ ID NO: 12 - DNA, 28 bp, Artificial Sequence
  Description of Artificial Sequence Synthetic primer
gtggtacccagcctcgcagacactcgac

SEQ ID NO: 13 - DNA, 30 bp, Artificial Sequence
  Description of Artificial Sequence Synthetic primer
cttgaagctttccttcctgctgtcgatgtt

SEQ ID NO: 14 - DNA, 30 bp, Artificial Sequence
  Description of Artificial Sequence Synthetic primer
ccattctagaatgcgtctgggaattggcac

SEQ ID NO: 15 - DNA, 29 bp, Artificial Sequence
  Description of Artificial Sequence Synthetic primer
gcaagcttgagctttcggacgaccattgc

SEQ ID NO: 16 - DNA, 30 bp, Artificial Sequence
  Description of Artificial Sequence Synthetic primer
gtgaattccggcagcgatcgaatgtattcg

SEQ ID NO: 17 - DNA, 20 bp, Artificial Sequence
  Description of Artificial Sequence Synthetic primer
accttccctgtgttttgcac

SEQ ID NO: 18 - DNA, 42 bp, Artificial Sequence
  Description of Artificial Sequence Synthetic primer
tgtgtttgtttccaagaaaagagggcattgaaggtgtagcat

SEQ ID NO: 19 - DNA, 42 bp, Artificial Sequence
  Description of Artificial Sequence Synthetic primer
atgctacaccttcaatgccctcttttcttggaaacaaacaca

SEQ ID NO: 20 - DNA, 20 bp, Artificial Sequence
  Description of Artificial Sequence Synthetic primer
tcattctagcttgcggtcct

SEQ ID NO: 21 - DNA, 20 bp, Artificial Sequence
  Description of Artificial Sequence Synthetic primer
ggcaacattgtcatgtctgg

SEQ ID NO: 22 - DNA, 20 bp, Artificial Sequence
  Description of Artificial Sequence Synthetic primer
gagcgaagcaagaatggaac

SEQ ID NO: 23 - DNA, 20 bp, Artificial Sequence
  Description of Artificial Sequence Synthetic primer
acaacctcggcttcaacatc

SEQ ID NO: 24 - DNA, 20 bp, Artificial Sequence
  Description of Artificial Sequence Synthetic primer
agcgacataggtggtgtcgt

SEQ ID NO: 25 - DNA, 19 bp, Artificial Sequence
  Description of Artificial Sequence Synthetic primer
cggtaccactggaaacact

SEQ ID NO: 26 - DNA, 20 bp, Artificial Sequence
  Description of Artificial Sequence Synthetic primer
tgggtgagaccagtaacacg

SEQ ID NO: 27 - DNA, 20 bp, Artificial Sequence
  Description of Artificial Sequence Synthetic primer
gtggcatgcaatatggacaa

SEQ ID NO: 28 - DNA, 20 bp, Artificial Sequence
  Description of Artificial Sequence Synthetic primer
gcggtagaaatttgccttga

SEQ ID NO: 29 - DNA, 19 bp, Artificial Sequence
  Description of Artificial Sequence Synthetic primer
cattctggggacgttccat

SEQ ID NO: 30 - DNA, 20 bp, Artificial Sequence
  Description of Artificial Sequence Synthetic primer
ctgttcgctccgaagttcat

SEQ ID NO: 31 - DNA, 19 bp, Artificial Sequence
  Description of Artificial Sequence Synthetic primer
acacagggtagaacccaac

SEQ ID NO: 32 - DNA, 20 bp, Artificial Sequence
  Description of Artificial Sequence Synthetic primer
gttttcccgctgtcaaagaa

SEQ ID NO: 33 - DNA, 20 bp, Artificial Sequence
  Description of Artificial Sequence Synthetic primer
ccacttgaccgtcacctaca

SEQ ID NO: 34 - DNA, 20 bp, Artificial Sequence
  Description of Artificial Sequence Synthetic primer
gtgttcttgctgttgccaaa

SEQ ID NO: 35 - DNA, 20 bp, Artificial Sequence
  Description of Artificial Sequence Synthetic primer
cgtagagaatgcccttccag

SEQ ID NO: 36 - DNA, 20 bp, Artificial Sequence
  Description of Artificial Sequence Synthetic primer
cgacacccttgatttggtct

SEQ ID NO: 37 - DNA, 399 bp, Alternaria brassicicola
atgcagttcaccaccgccatcttcgctctcgctgctgcgactgccgccaacgccgcttcaacctacggtgccttcaacgtcaccgtcgagagctctagctacgccaacggcgtcacctcgcgcaccgtcctctccgactactctggcgacgccgctatccacgccgtctgcaagtacgagttcaaccctgctgccgagcccaaggagacctccagctgcgagcccaactctttctcctacgagtacgatggccagaccatcaaggtccagcagattgtcgagaagcccaaccccatgaccgtcttcggtgaggctcctcttgccctcactaccgagggcggcgctggcaggacctccaagggtaacgccatcttcgacgccaccagtgccatcgcttaa

SEQ ID NO: 38 - protein, 6 aa, Artificial Sequence
  Description of Artificial Sequence Synthetic peptide
HHHHHH